(19) United States
(12) Patent Application Publication
(10) Pub. No.: US 2026/0241038 A1
(43) Pub. Date: Aug. 20, 2026

(54) METHOD FOR IMPROVING NEUROLOGICAL FUNCTION IN MPSI AND MPSII AND OTHER NEUROLOGICAL DISORDERS

(71) Applicants: Regents of the University of Minnesota, Minneapolis, MN (US); REGENXBIO Inc., Rockville, MD (US)

(72) Inventors: Kanut Laoharawee, Minneapolis, MN (US); Kelly M. Podetz-Pedersen, Chaska, MN (US); Karen Kozarsky, Bala Cynwyd, PA (US); R. Scott Mclvor, St. Louis Park, MN (US); Lalitha R. Belur, St. Paul, MN (US)

(21) Appl. No.: 19/331,876

(22) Filed: Sep. 17, 2025

Related U.S. Application Data
(62) Division of application No. 17/508,714, filed on Oct. 22, 2021, now abandoned.
Application No. 17/508,714 is a continuation of application No. 16/461,271, filed on Nov. 15, 2017, now abandoned.
(60) Provisional application No. 62/422,453, filed on Nov. 15, 2016.
(60) Provisional application No. 62/422,436, filed on Nov. 15, 2016.
(60) Provisional application No. 62/458,248, filed on Feb. 13, 2017.
(60) Provisional application No. 62/458,259, filed on Feb. 13, 2017.

(51) Int. Cl. A61K 48/00; A61K 9/00; A61K 38/46; A61K 38/47; A61K 45/06; A61P 25/28; C12N 7/00

(52) U.S. Cl. CPC A61K 48/0075; A61K 9/0019; A61K 9/0085; A61K 38/465; A61K 38/47; A61K 45/06; A61P 25/28; C12N 7/00; C12Y 301/06013; C12Y 302/01076; C12N 2320/32; C12N 2750/14143

(57) ABSTRACT

A method to prevent, inhibit or treat one or more neurological symptoms associated with a central nervous system disorder, e.g. MPSI or MPSII by, for example, intrathecally, intracerebroventricularly or intravenously administering a rAAV encoding a gene product associated with the disease, e.g., administering to an adult mammal in which the gene product is absent, defective or present at a reduced level relative to a mammal without the disease.

### CROSS-REFERENCE TO RELATED APPLICATIONS

This application is a divisional of U.S. application Ser. No. 17/508,714, filed Oct. 22, 2021, which is a continuation of U.S. application Ser. No. 16/461,271, filed May 15, 2019, which is a U.S. National Stage Application under 35 U.S.C. 371 from International Application Serial No. PCT/US2017/061838, filed on Nov. 15, 2017, which claims the benefit of the filing date of U.S. application Ser. No. 62/458,248, filed on Feb. 13, 2017, U.S. application Ser. No. 62/458,259, filed on Feb. 13, 2017, U.S. application Ser. No. 62/422,453, filed on Nov. 15, 2016, and U.S. application Ser. No. 62/422,436, filed on Nov. 15, 2016, the disclosures of which are incorporated by reference herein.

### STATEMENT OF GOVERNMENT RIGHTS

This invention was made with government support under HD032652 awarded by the National Institutes of Health. The Government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Feb. 23, 2026, is named 600A18US3.xml and is 8,192 bytes in size.

### BACKGROUND

The mucopolysaccharidoses (MPSs) are a group of 11 storage diseases caused by disruptions in glycosaminoglycan (GAG) catabolismleading to their accumulation in lysosomes (Muenzer, 2004; Munoz-Rojas et al., 2008). Manifestations of varying severity include organomegaly, skeletal dysplasias, cardiac and pulmonary obstruction and neurological deterioration. For MPS I, deficiency of iduronidase (IDUA), severity ranges from mild (Scheie syndrome) to moderate (Hurler-Scheie) to severe (Hurler syndrome), with the latter resulting in neurologic deficiency and death by age 15 (Muenzer, 2004; Munoz-Rojas et al., 2008). Therapies for MPSs have been for the most part palliative. However, there are some of the MPS diseases, including Hurler syndrome, for which allogeneic hematopoietic stem cell transplantation (HSCT) has exhibited efficacy (Krivit, 2004; Orchard et al., 2007; Peters et al., 2003). Additionally, for more and more of the MPS diseases, enzyme replacement therapy (ERT) is becoming available (Brady, 2006). In general, HSCT and ERT result in the clearing of storage materials and improved peripheral conditions, although some problems persist after treatment (skeletal, cardiac, corneal clouding). The primary challenge in these cellular and enzyme therapies is effectiveness in addressing neurological manifestations, as peripherally administered enzyme does not penetrate the blood-brain barrier and HSCT has been found to be of benefit for some, but not all, MPS's.

MPS I has been one of the most extensively studied of the MPS diseases for development of cellular and molecular therapies. The effectiveness of allogeneic HSCT is most likely the result of metabolic cross-correction, whereby the missing enzyme is released from donor-derived cells and subsequently taken up by host cells and trafficked to lysosomes, where the enzyme contributes to lysosomal metabolism (Fratantoni et al., 1968). Clearing of GAG storage materials is subsequently observed in peripheral organs such as liver and spleen, there is relief from cardiopulmonary obstruction and improvement in corneal clouding (Orchard et al., 2007). Of particular importance is the effect of allogeneic stem cell transplantation on the emergence of neurologic manifestations in the MPS diseases. In this regard, there is evidence for several MPS diseases that individuals engrafted with allogeneic stem cells face an improved outcome in comparison with untransplanted patients (Bjoraker et al., 2006; Krivit, 2004; Orchard et al., 2007; Peters et al., 2003). A central hypothesis explaining the neurologic benefit of allogeneic hematopoietic stem cell transplant is the penetration of donor-derived hematopoietic cells (most likely microglia) (Hess et al., 2004; Unger et al., 1993) into the central nervous system, where the missing enzyme is expressed by engrafted cells from which point the enzyme diffuses into CNS tissues and participates in clearing of storage materials. The level of enzyme provided to CNS tissues is thus limited to that amount expressed and released from donor-derived cells engrafting in the brain. While such engraftment is of great benefit for MPS I, recipients nonetheless continue to exhibit below normal IQ and impaired neurocognitive capability (Ziegler and Shapiro, 2007).

The phenomenon of metabolic cross correction also explains the effectiveness of ERT for several lysosomal storage diseases (Brady, 2006), most notably MPS I. However, due to the requirement for penetration of the blood-brain barrier (BBB) by the enzyme missing in the particular lysosomal storage disease (LSD) in order to effectively reach the CNS, effectiveness of enzyme therapy for neurologic manifestations of lysosomal storage disease (LSD) has not been observed (Brady, 2006). Enzymes are almost always too large and generally too charged to effectively cross the BBB. This has prompted investigations into invasive intrathecal enzyme administration (Dickson et al., 2007), for which effectiveness has been demonstrated in a canine model of MPS I (Kakkis et al., 2004) and for which human clinical trials are beginning for MPS I (Pastores, 2008; Munoz-Rojas et al., 2008). Key disadvantages of enzyme therapy include its great expense (>$200,000 per year) and the requirement for repeated infusions of recombinant protein. Current clinical trials of intrathecal IDUA administration are designed to inject the enzyme only once every three months, so the effectiveness of this dosing regimen remains uncertain.

Another MPS, Hunter Syndrome (Mucopolysaccharidosis type II; MPS II), is an X-linked recessive inherited lysosomal disease caused by deficiency of iduronate-2-sulfatase (IDS) characterized by accumulation of glycosaminoglycans (GAGs) in tissues, resulting in skeletal dysplasias, hepatosplenomegaly, cardiopulmonary obstruction, and neurologic deterioration. Patient standard of care is enzyme replacement therapy (ERT) although ERT is not associated with neurologic improvement. There is currently no existing accepted therapy for neurologic manifestations of MPS II (Hunter syndrome).

### SUMMARY

In a mouse model of IDS deficiency, intracerebroventricular (ICV) administration of AAV9.hIDS into young 8-week old mice resulted in corrective levels of hIDS enzyme activity, reduction of GAG storage to near wild-type (WT)-levels and prevention of neurocognitive dysfunction, compared to IDS deficient control littermates. Since the emergence of neurologic manifestations in young adults could be prevented, it was hypothesized that older adult MPS II animals treated at 2 or 4 months of age by ICV administration of AAV9.hIDS may recover neurobehavioral function and show corrected levels of IDS enzyme activity and GAG storage. As disclosed herein, by 4 weeks post-ICV injection, IDS enzyme activity in the circulation was 1000-times that of WT-levels (305+/−85nmol/hr/mL compared to 0.39+/−0.04nmol/hr/ml). At 36 weeks of age, the treated animals were tested for neurocognitive function in the Barnes maze. Performance of the treated animals was indistinguishable from that of unaffected littermates and significantly improved compared to untreated MPS II mice. Cognitive function that is lost by 4 months of age can thus be restored in MPS II mice by delivery of AAV9 encoding IDS to the cerebrospinal fluid. The exciting implication of these results is the prospect that human MPS II may be treatable after the development of neurologic manifestations by AAV mediated IDS gene transfer to the CNS. Thus, adeno-associated virus mediated IDS gene transfer to the CNS prevented the development of neurologic dysfunction in a murine model of MPS II. Remarkably, AAV mediated IDS gene transfer to the CNS also resulted in the recovery of neurologic function when administered to animals that have already developed manifestations of the disease. Thus, recovery of neurologic function in MPS II can be achieved by IDS gene transfer into the CNS after manifestations of neurologic deficiency have already emerged and so patients with Hunter syndrome may be treated in this manner even after development of neurologic symptoms.

In one embodiment, the invention provides for delivery to the CNS of therapeutic proteins via AAV to prevent, inhibit or treat neurocognitive dysfunction, enhance neurocognition, recover neurologic function or prevent neurocognitive deterioration in a mammal. In one embodiment, the mammal has or at risk of having, e.g., is pre-symptomatic, MPSII. In one embodiment, the mammal has or at risk of having, e.g., pre-symptomatic, MPSI. In one embodiment, rAAV is delivered to a mammal intrathecally (IT), endovascularly (IV), or intracerebroventricularly (ICV) to prevent, inhibit or treat neurocognitive dysfunction or restore (enhance) neurocognitive function. In one embodiment, the mammal is subjected to immunosuppression. In one embodiment, the mammal is subjected to tolerization. In one embodiment, methods of preventing, inhibiting, and/or treating neurocognitive dysfunction in, for example, an adult mammal, are provided. In one embodiment, the mammal is subjected to tolerization. In one embodiment, methods of preventing, inhibiting, and/or treating neurocognitive dysfunction in, for example, a non-adult mammal, are provided. In one embodiment, the rAAV is administered to an infant (e.g., a human that is 3 years old or less such as less than 3, 2.5, 2, or 1.5 years of age), a pre-adolescent (e.g., in humans those less than 10, 9, 8, 7, 6, 5, or 4 but greater than 3 years of age), or adult (e.g., humans older than about 12 years of age).

In one embodiment, the methods involve delivering to the CNS of a mammal in need of treatment a composition comprising an effective amount of a recombinant adeno-associated virus (rAAV) vector comprising an open reading frame encoding IDS. The AAV vector can be administered in a variety of ways to ensure that it is delivered to the CNS/brain, and that the transgene is successfully transduced in the subject's CNS/brain. Routes of delivery to the CNS/brain include, but are not limited to intrathecal administration (e.g., via the cisterna magna or via lumbar puncture), intracranial administration, e.g., intracerebroventricular administration, or lateral cerebroventricular administration, endovascular administration, and intraparenchymal administration. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the mammal relative to a corresponding mammal with MPSII that is not administered the AAV-IDS. In one embodiment, a patient having or at risk of having MPSII, e.g., a human from about 4 months of age to about 5 years of age, is administered about 1.3×10¹⁰ genome copies (GC)/g brain mass to about 6.5×10¹⁰ GC/g brain mass. In one embodiment, a patient having or at risk of having MPSII, e.g., a human from ≥4 to about 9 months of age is administered about 7.8×10¹² flat dose to about 3.9×10¹³ flat dose; from >9 to about 18 months of age is administered about 1.3×10¹³ flat dose to about 6.5×10¹³ flat dose; from about ≥18 mo to about ≤3 years of age is administered about 1.4×10¹³ to about 7.2×10¹³ flat dose; or ≥3 years of age is administered about 1.7×10¹³ to about 8.5×10¹³ flat dose, e.g., intrathecally, and optionally via the cisterna magna or via lumbar puncture. The dose can be in a volume of about 5 to about 20 mL.

In one embodiment, the methods involve delivering to the CNS of an adult mammal in need of treatment a composition comprising an effective amount of a rAAV serotype 9 (rAAV9) vector comprising an open reading frame encoding IDS. In one embodiment, the methods involve delivering to the CNS of an adult mammal in need of treatment a composition comprising an effective amount of a rAAV9 vector comprising an open reading frame encoding IDS and one encoding SUMF-1. For example, AAV9-IDS may be administered by direct injection into the lateral ventricles of adult IDS-deficient mice that are either immunocompetent, immunodeficient, immunosuppressed, e.g., with cyclophosphamide (CP), or immunotolerized by injection of IDS protein. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

Thus, the invention includes the use of recombinant AAV (rAAV) vectors that encode a gene product with therapeutic effects when expressed in the CNS of a mammal. In one embodiment, the mammal is an immunocompetent mammal with a disease or disorder of the CNS (a neurologic disease). An “immunocompetent” mammal as used herein is a mammal of an age where both cellular and humoral immune responses are elicited after exposure to an antigenic stimulus, by upregulation of Th1 functions or IFN-γ production in response to polyclonal stimuli, in contrast to a neonate which has innate immunity and immunity derived from the mother, e.g., during gestation or via lactation. An adult mammal that does not have an immunodeficiency disease is an example of an immunocompetent mammal. For example, an immunocompetent human is typically at least 1, 2, 3, 4, 5 or 6 months of age, and includes adult humans without an immunodeficiency disease. In one embodiment, the AAV is administered intrathecally. In one embodiment, the AAV is administered intracranially (e.g., intracerebroventricularly). In one embodiment, the AAV is administered, with or without a permeation enhancer. In one embodiment, the AAV is administered endovascularly, e.g., carotid artery administration, with or without a permeation enhancer.

In one embodiment, the mammal that is administered the AAV is immunodeficient or is subjected to immunotolerization or immune suppression, e.g., to induce higher levels of therapeutic protein expression relative to a corresponding mammal that is administered the AAV but not subjected to immunotolerization or immune suppression. In one embodiment, an immune suppressive agent is administered to induce immune suppression. In one embodiment, the mammal that is administered the AAV is not subjected to immunotolerization or immune suppression (e.g., administration of the AAV alone provides for the therapeutic effect). In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the invention provides a method to augment secreted protein in a mammal having neurological disease, which may include a neurocognitive dysfunction. The method includes administering to the mammal a composition comprising an effective amount of a recombinant adeno-associated virus (rAAV) vector comprising an open reading frame encoding the secreted protein, the expression of which in the mammal enhances neurocognition relative to a mammal with the disease or dysfunction but not administered the rAAV. In one embodiment, the rAAV or a different rAAV encodes a neuroprotective protein, e.g., GDNF or Neurturin. In one embodiment, the rAAV or a different rAAV encodes an antibody. In one embodiment, the mammal is not treated with an immunosuppressant. In another embodiment, for example, in subjects that may generate an immune response that neutralizes activity of the therapeutic protein, the mammal is treated with an immunosuppressant, e.g., a glucocorticoid, cytostatic agents including an alkylating agent, an anti-metabolite, a cytotoxic antibiotic, an antibody, or an agent active on immunophilin, such as a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In one embodiment, the rAAV and the immune suppressant are co-administered or the immune suppressant is administered after the rAAV. In one embodiment, the immune suppressant is intrathecally administered. In one embodiment, the immune suppressant is intracerebroventricularly administered. In one embodiment, the rAAV vector is a rAAV1, rAAV3, rAAV4, rAAV5, rAA rh10, or rAAV9 vector. In one embodiment, prior to administration of the composition the mammal is immunotolerized. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the invention provides a method to prevent, inhibit or treat neurocognitive dysfunction in a mammal. The method includes administering to the mammal a composition comprising an effective amount of a recombinant adeno-associated virus (rAAV) vector comprising an open reading frame encoding an IDS, the expression of which in the mammal prevents, inhibits or treats neurocognitive dysfunction. In one embodiment, the amount of AAV-IDUA administered results in an increase, e.g., 2—, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, a method to enhance or restore neurocognitive function in a mammal with MPSII is provided. The method includes intrathecally, e.g., to the lumbar region, or intracerebroventricularly, e.g., to the lateral ventricle, administering to the mammal a composition comprising an effective amount of a rAAV vector comprising an open reading frame encoding an IDS, the expression of which in the central nervous system of the mammal enhances or restores neurocognitive function. In one embodiment, the mammal is an immunocompetent adult mammal. In one embodiment, the mammal is an immunocompetent non-adult mammal. In one embodiment, the rAAV vector is an AAV1, AAV2, AAV3, AAV4, AAV5, AAV6, AAV7, AAV8, AAVrh10, or AAV9 vector. In one embodiment, the mammal is a human. In one embodiment, multiple doses are administered. In one embodiment, the composition is administered weekly, monthly or two or more months apart. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the method includes intrathecally, e.g., to the cisterna magna or to the lumbar cistern, administering to a mammal a composition comprising an effective amount of a rAAV vector comprising an open reading frame encoding an IDS, the expression of which in the central nervous system of the mammal restores or enhances neurocognitive function, and optionally administering a permeation enhancer. In one embodiment, the permeation enhancer is administered before the composition. In one embodiment, the composition comprises a permeation enhancer. In one embodiment, the permeation enhancer is administered after the composition. In one embodiment, the mammal is an immunocompetent adult. In one embodiment, the rAAV vector is an AAV-1, AAV-3, AAV-4, AAV-5, AAV-6, AAV-7, AAV-8, AAV rh10, or AAV-9 vector. In one embodiment, the mammal is a human. In one embodiment, multiple doses are administered. In one embodiment, the composition is administered weekly, monthly or two or more months apart. In one embodiment, the mammal that is intrathecally administered the AAV is not subjected to immunotolerization or immune suppression (e.g., administration of the AAV alone provides for the therapeutic effect). In one embodiment, the mammal that is intrathecally administered the AAV is immunodeficient or is subjected to immunotolerization or immune suppression, e.g., to induce higher levels of therapeutic protein expression relative to a corresponding mammal that is intrathecally administered the AAV but not subjected to immunotolerization or immune suppression. In one embodiment, the amount of AAV-IDUA administered results in an increase, e.g., 2-, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS. In one embodiment, the method includes intracerebroventricularly, e.g., to the lateral ventricle, administering to an immunocompetent mammal a composition comprising an effective amount of a rAAV vector comprising an open reading frame encoding an IDS, the expression of which in the central nervous system of the mammal enhances or restores neurocognitive function. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly, monthly or two or more months apart. In one embodiment, the mammal that is intracerebroventricularly administered the AAV is not subjected to immunotolerization or immune suppression (e.g., administration of the AAV alone provides for the therapeutic effect). In one embodiment, the mammal that is intracerebroventricularly administered the AAV is immunodeficient or is subjected to immunotolerization or immune suppression, e.g., to induce higher levels of therapeutic protein expression relative to a corresponding mammal that is intracerebroventricularly administered the AAV but not subjected to immunotolerization or immune suppression In one embodiment, the mammal is immunotolerized to the gene product before the composition comprising the AAV is administered. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

Further provided is a method to enhance or restore neurocognitive function associated with MPSII in a mammal. The method includes endovascularly administering to the mammal a composition comprising an effective amount of a rAAV vector comprising an open reading frame encoding an IDS, the expression of which in the central nervous system of the mammal, and optionally an effective amount of a permeation enhancer. In one embodiment, the composition comprises the permeation enhancer. In one embodiment, the permeation enhancer comprises mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA. In one embodiment, the mammal is an immunocompetent adult. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly. In one embodiment, the composition is administered weekly, monthly or two or more months apart. In one embodiment, the mammal that is endovascularly administered the AAV is not subjected to immunotolerization or immune suppression (e.g., administration of the AAV provides for the therapeutic effect). In one embodiment, the mammal that is endovascularly administered the AAV is immunodeficient or is subjected to immunotolerization or immune suppression, e.g., to induce higher levels of therapeutic protein expression relative to a corresponding mammal that is endovascularly administered the AAV but not subjected to immunotolerization or immune suppression. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the method includes administering to an adult mammal a composition comprising an effective amount of a rAAV9 vector comprising an open reading frame encoding an IDS, the expression of which in the central nervous system of the mammal enhances or restores neurocognitive function, and optionally administering a permeation enhancer In one embodiment, the permeation enhancer is administered before the composition. In one embodiment, the composition comprises a permeation enhancer. In one embodiment, the permeation enhancer is administered after the composition. In one embodiment, the mammal is an immunocompetent adult. In one embodiment, the mammal is a human. In one embodiment, multiple doses are administered. In one embodiment, the composition is administered weekly, monthly or two or more months apart. In one embodiment, the mammal that is administered the AAV is not subjected to immunotolerization or immune suppression. In one embodiment, the mammal that is administered the AAV is subjected to immunotolerization or immune suppression, e.g., to induce higher levels of IDUA protein expression relative to a corresponding mammal that is administered the AAV but not subjected to immunotolerization or immune suppression. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the methods described herein involve delivering to the CNS of an immunocompetent adult human in need of treatment a composition comprising an effective amount of a rAAV9 vector comprising an open reading frame encoding an IDS. Routes of administration to the CNS/brain include, but are not limited to intrathecal administration, intracranial administration, e.g., intracerebroventricular administration or lateral cerebroventricular administration, administration, endovascular administration, and intraparenchymal administration. In one embodiment, the amount of AAV-IDUA administered results in an increase, e.g., 2—, 5—, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In a mouse model of IDUA deficiency, intracerebroventricular (ICV) administration of AAV9.hIDUA resulted in recovery of neurologic function after the manifestations of neurologic deficiency had already emerged. Thus, remarkably, AAV mediated IDUA gene transfer to the CNS resulted in the recovery of neurologic function when administered to animals that have already developed manifestations of the disease. Therefore, patients with with MPS I disorders, e.g., Hurler syndrome, Hurler-Scheie syndrome or Scheie syndrome, may be treated in this manner even after development of neurologic symptoms.

In one embodiment, the invention provides for delivery to the CNS of therapeutic proteins via AAV to prevent, inhibit or treat neurocognitive dysfunction in a mammal having MPS I. In one embodiment, rAAV is delivered to a mammal intrathecally (IT), e.g., via the cisterna magna or by lumbar puncture, endovascularly (IV), or cerebroventricularly (ICV) to prevent, inhibit or treat neurocognitive dysfunction or restore (enhance) neurocognitive function. In one embodiment, the mammal is subjected to immunosuppression. In one embodiment, the mammal is subjected to tolerization. In one embodiment, methods of preventing, inhibiting, and/or treating neurocognitive dysfunction in, for example, an adult mammal, are provided. The methods involve delivering to the CNS of a mammal in need of treatment a composition comprising an effective amount of a recombinant adeno-associated virus (rAAV) vector comprising an open reading frame encoding IDUA. The AAV vector can be administered in a variety of ways to ensure that it is delivered to the CNS/brain, and that the transgene is successfully transduced in the subject's CNS/brain. Routes of delivery to the CNS/brain include, but are not limited to intrathecal administration, intracranial administration, e.g., intracerebroventricular administration, or lateral cerebroventricular administration, administration, endovascular administration, and intraparenchymal administration.

In one embodiment, the methods involve delivering to the CNS of an adult mammal in need of treatment a composition comprising an effective amount of a rAAV serotype 9 (rAAV9) vector comprising an open reading frame encoding IDUA. In one embodiment, the methods involve delivering to the CNS of an adult mammal in need of treatment a composition comprising an effective amount of a rAAV9 vector comprising an open reading frame encoding IDUA and optionally another open reading frame. For example, AAV9-IDUA may be administered by direct injection into the lateral ventricles of adult IDUA-deficient mice that are either immunocompetent, immunodeficient, immunosuppressed, e.g., with cyclophosphamide (CP), or immunotolerized by injection of IDUA protein. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

Thus, the invention includes the use of recombinant AAV (rAAV) vectors that encode a gene product with therapeutic effects when expressed in the CNS of a mammal. In one embodiment, the mammal is an immunocompetent mammal with a disease or disorder of the CNS (a neurologic disease). An “immunocompetent” mammal as used herein is a mammal of an age where both cellular and humoral immune responses are elicited after exposure to an antigenic stimulus, by upregulation of Th1 functions or IFN-γ production in response to polyclonal stimuli, in contrast to a neonate which has innate immunity and immunity derived from the mother, e.g., during gestation or via lactation. An adult mammal that does not have an immunodeficiency disease is an example of an immunocompetent mammal. For example, an immunocompetent human is typically at least 1, 2, 3, 4, 5 or 6 months of age, and includes adult humans without an immunodeficiency disease. In one embodiment, the AAV is administered intrathecally. In one embodiment, the AAV is administered intracranially (e.g., intracerebroventricularly). In one embodiment, the AAV is administered, with or without a permeation enhancer. In one embodiment, the permeation enhancer comprises mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA. In one embodiment, the AAV is administered endovascularly, e.g., carotid artery administration, with or without a permeation enhancer. In one embodiment, the mammal that is administered the AAV is immunodeficient or is subjected to immunotolerization or immune suppression, e.g., to induce higher levels of therapeutic protein expression relative to a corresponding mammal that is administered the AAV but not subjected to immunotolerization or immune suppression. In one embodiment, an immune suppressive agent is administered to induce immune suppression. In one embodiment, the mammal that is administered the AAV is not subjected to immunotolerization or immune suppression (e.g., administration of the AAV alone provides for the therapeutic effect). In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

In one embodiment, the invention provides a method to augment secreted protein in a mammal having neurological disease, which may include a neurocognitive dysfunction. The method includes administering to the mammal a composition comprising an effective amount of a recombinant adeno-associated virus (rAAV) vector comprising an open reading frame encoding the secreted protein, the expression of which in the mammal enhances neurocognition relative to a mammal with the disease or dysfunction but not administered the rAAV. In one embodiment, the rAAV or a different rAAV encodes a neuroprotective protein, e.g., GDNF or Neurturin. In one embodiment, the rAAV or a different rAAV encodes an antibody. In one embodiment, the mammal is not treated with an immunosuppressant. In another embodiment, for example, in subjects that may generate an immune response that neutralizes activity of the therapeutic protein, the mammal is treated with an immunosuppressant, e.g., a glucocorticoid, cytostatic agents including an alkylating agent, an anti-metabolite, a cytotoxic antibiotic, an antibody, or an agent active on immunophilin, such as a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In one embodiment, the rAAV and the immune suppressant are co-administered or the immune suppressant is administered after the rAAV. In one embodiment, the immune suppressant is intrathecally administered. In one embodiment, the immune suppressant is intracerebroventricularly administered. In one embodiment, the rAAV vector is a rAAV1, rAAV3, rAAV4, rAAV5, rAA rh10, or rAAV9 vector. In one embodiment, prior to administration of the composition the mammal is immunotolerized. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

In one embodiment, the invention provides a method to prevent, inhibit or treat neurocognitive dysfunction in a mammal. The method includes administering to the mammal a composition comprising an effective amount of a recombinant adeno-associated virus (rAAV) vector comprising an open reading frame encoding an IDUA, the expression of which in the mammal prevents, inhibits or treats neurocognitive dysfunction. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the mammal, e.g., a non-adult mammal, relative to a corresponding mammal with MPSI that is not administered the AAV-IDUA. In one embodiment, a MPS I patient≥6 years old is treated with an amount of rAAV-IDUA effective to prevent, inhibit or treat neurocognitive dysfunction. In one embodiment, a MPS I patient≤2 years old is treated with an amount of rAAV effective to prevent, inhibit or treat neurocognitive dysfunction. In one embodiment, the mammal, e.g., human, is administered about 1×10¹² to about 2×10¹⁴ genome copies (GC) (flat dose), about 5×10¹² to about 2×10¹⁴ GC flat dose; about 1×10¹³ to about 1×10¹⁴ GC flat dose; about 1×10¹³ to about 2×10¹³ GC flat dose; or about 6×10¹³ to about 8×10¹³ GC flat dose, e.g., administered intrathecally, for example, via the cisterna magna or by lumbar puncture. In one embodiment, a non-adult MPS1 patient is administered about 1×10¹³ to about 5.6×10¹³ GC flat dose. In one embodiment, an adult MPSI patient is administered about 1×10¹² to about 5.6×10¹³ GC flat dose. In one embodiment, for a MPSI patient≥6 years old, a single flat dose is administered IC: either a dose of 2×10⁹ GC/g brain mass (2.6×10¹² GC), or a dose of 1×10¹⁰ GC/g brain mass (1.3×10¹³ GC). The dose can be in a volume of about 5 to about 20 mL.

In one embodiment, a method to enhance or restore neurocognitive function in a mammal with MPS I is provided. The method includes intrathecally, e.g., to the lumbar region, or intracerebroventricularly, e.g., to the lateral ventricle, administering to the mammal a composition comprising an effective amount of a rAAV vector comprising an open reading frame encoding an IDUA, the expression of which in the central nervous system of the mammal enhances or restores neurocognitive function. In one embodiment, the mammal is an immunocompetent adult. In one embodiment, the rAAV vector is an AAV1, AAV2, AAV3, AAV4, AAV5, AAV6, AAV7, AAV8, AAVrh10, or AAV9 vector. In one embodiment, the mammal is a human. In one embodiment, multiple doses are administered. In one embodiment, the composition is administered weekly, monthly or two or more months apart. In one embodiment, the amount of AAV-IDUA administered results in an increase, e.g., 2-, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

In one embodiment, the method includes intrathecally, e.g., to the cisterna magna or to the lumbar cistern, administering to a mammal a composition comprising an effective amount of a rAAV vector comprising an open reading frame encoding an IDUA, the expression of which in the central nervous system of the mammal restores or enhances neurocognitive function, and optionally administering a permeation enhancer. In one embodiment, the permeation enhancer is administered before the composition. In one embodiment, the composition comprises a permeation enhancer. In one embodiment, the permeation enhancer is administered after the composition. In one embodiment, the mammal is an immunocompetent adult. In one embodiment, the rAAV vector is an AAV-1, AAV-3, AAV-4, AAV-5, AAV-6, AAV-7, AAV-8, AAV rh10, or AAV-9 vector. In one embodiment, the mammal is a human. In one embodiment, multiple doses are administered. In one embodiment, the composition is administered weekly, monthly or two or more months apart. In one embodiment, the mammal that is intrathecally administered the AAV is not subjected to immunotolerization or immune suppression (e.g., administration of the AAV alone provides for the therapeutic effect). In one embodiment, the mammal that is intrathecally administered the AAV is immunodeficient or is subjected to immunotolerization or immune suppression, e.g., to induce higher levels of therapeutic protein expression relative to a corresponding mammal that is intrathecally administered the AAV but not subjected to immunotolerization or immune suppression. In one embodiment, the amount of AAV-IDUA administered results in an increase, e.g., 2-, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

In one embodiment, the method includes intracerebroventricularly, e.g., to the lateral ventricle, administering to an immunocompetent mammal a composition comprising an effective amount of a rAAV vector comprising an open reading frame encoding an IDUA, the expression of which in the central nervous system of the mammal enhances or restores neurocognitive function. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly, monthly or two or more months apart. In one embodiment, the mammal that is intracerebroventricularly administered the AAV is not subjected to immunotolerization or immune suppression (e.g., administration of the AAV alone provides for the therapeutic effect). In one embodiment, the mammal that is intracerebroventricularly administered the AAV is immunodeficient or is subjected to immunotolerization or immune suppression, e.g., to induce higher levels of therapeutic protein expression relative to a corresponding mammal that is intracerebroventricularly administered the AAV but not subjected to immunotolerization or immune suppression In one embodiment, the mammal is immunotolerized to the gene product before the composition comprising the AAV is administered. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

Further provided is a method to enhance or restore neurocognitive function associated With MPS I in a mammal. The method includes endovascularly administering to the mammal a composition comprising an effective amount of a rAAV vector comprising an open reading frame encoding an IDUA, the expression of which in the central nervous system of the mammal, and optionally an effective amount of a permeation enhancer. In one embodiment, the composition comprises the permeation enhancer. In one embodiment, the permeation enhancer comprises mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA. In one embodiment, the mammal is an immunocompetent adult. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly. In one embodiment, the composition is administered weekly, monthly or two or more months apart. In one embodiment, the mammal that is endovascularly administered the AAV is not subjected to immunotolerization or immune suppression (e.g., administration of the AAV provides for the therapeutic effect). In one embodiment, the mammal that is endovascularly administered the AAV is immunodeficient or is subjected to immunotolerization or immune suppression, e.g., to induce higher levels of therapeutic protein expression relative to a corresponding mammal that is endovascularly administered the AAV but not subjected to immunotolerization or immune suppression. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

In one embodiment, the method includes administering to an adult mammal a composition comprising an effective amount of a rAAV9 vector comprising an open reading frame encoding an IDUA, the expression of which in the central nervous system of the mammal enhances or restores neurocognitive function, and optionally administering a permeation enhancer In one embodiment, the permeation enhancer is administered before the composition. In one embodiment, the composition comprises a permeation enhancer. In one embodiment, the permeation enhancer is administered after the composition. In one embodiment, the mammal is an immunocompetent adult. In one embodiment, the mammal is a human. In one embodiment, multiple doses are administered. In one embodiment, the composition is administered weekly, monthly or two or more months apart. In one embodiment, the mammal that is administered the AAV is not subjected to immunotolerization or immune suppression. In one embodiment, the mammal that is administered the AAV is subjected to immunotolerization or immune suppression, e.g., to induce higher levels of IDUA protein expression relative to a corresponding mammal that is administered the AAV but not subjected to immunotolerization or immune suppression.

In one embodiment, the methods described herein involve delivering to the CNS of an immunocompetent adult human in need of treatment a composition comprising an effective amount of a rAAV9 vector comprising an open reading frame encoding an IDUA. Routes of administration to the CNS/brain include, but are not limited to intrathecal administration, intracranial administration, e.g., intracerebroventricular administration or lateral cerebroventricular administration, administration, endovascular administration, and intraparenchymal administration. In one embodiment, the amount of AAV-IDUA administered results in an increase, e.g., 2—, 5—, 10-, 25-, 50-, 100- or 200-fold or more, up to 1000-fold more IDUA, e.g., in plasma or the brain, in the adult mammal relative to a corresponding mammal with MPSI that is not administered the AAV-IDUA.

Diseases that may exhibit neurologic symptoms or neurocognitive dysfunction that may be prevented, inhibited or treated using the methods disclosed herein include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy (a deficiency of survivor of motor neuron-1, SMN-1), Spinocerebellar ataxia, Tangier disease, Tay-Sachs disease, Tuberous sclerosis, Von Hippel-Lindau syndrome, Williams syndrome, Wilson's disease, or Zellweger syndrome. In one embodiment, the disease is a lysosomal storage disease, e.g., a lack or deficiency in a lysosomal storage enzyme. Lysosomal storage diseases include, but are not limited to, mucopolysaccharidosis (MPS) diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA: alpha-glucosaminide N-acetyl transferase or N-acetylglucosamine-6-sulfate sulfatase); mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA: alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), neuronal ceroid lipofuscinosis (NCL) (a deficiency of ceroid lipofucinoses (CLNs), e.g., Batten disease having a deficiency in the gene product of one or more of CLN1 to CLN14), and Gaucher disease (types I, II and III; a deficiency in glucocerebrosidase), as well as disorders such as Hermansky-Pudlak syndrome; Amaurotic idiocy; Tangier disease; aspartylglucosaminuria; congenital disorder of glycosylation, type la; Chediak-Higashi syndrome; macular dystrophy, corneal, 1; cystinosis, nephropathic; Fanconi-Bickel syndrome; Farber lipogranulomatosis; fibromatosis; geleophysic dysplasia; glycogen storage disease I; glycogen storage disease Ib; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder; arylsulfatase E disorder; ATPase, H+transporting, lysosomal, subunit 1 disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria. Replacement of less than 20%, e.g., less than 10% or about 1% to 5% levels of lysosomal storage enzyme found in nondiseased mammals, may prevent, inhibit or treat neurological symptoms such as neurological degeneration in mammals. In one embodiment, the disease to be prevented, inhibited or treated with a particular gene includes, but is not limited to, MPS I (IDUA), MPS II (IDS), MPS IIIA (Heparan-N-sulfatase; sulfaminidase), MPS IIIB (alpha-N-acetyl-glucosaminidase), MPS IIIC (Acetyl-CoA: alpha-N-acetyl-glucosaminide acetyltransferase), MPS IIID (N-acetylglucosamine 6-sulfatase), MPS VII (beta-glucoronidase), Gaucher (acid beta-glucosidase), Alpha-mannosidosis (alpha-mannosidase), Beta-mannosidosis (beta-mannosidase), Alpha-fucosidosis (alpha-fucosidase), Sialidosis (alpha-sialidase), Galactosialidosis (Cathepsin A), Aspartylglucosaminuria (aspartylglucosaminidase), GM1-gangliosidosis (beta-galactosidase), Tay-Sachs (beta-hexosaminidase subunit alpha), Sandhoff (beta-hexosaminidase subunit beta), GM2-gangliosidosis/variant AB (GM2 activator protein), Krabbe (galactocerebrosidase), Metachromatic leukodystrophy (arylsulfatase A), and other neurologic disorders including but not limited to Alzheimer's disease (expression of an antibody, such as an antibody to beta-amyloid, or an enzyme that attacks the plaques and fibrils associated with Alzheimer's), or Alzheimer's and Parkinson's diseases (expression of neuroprotective proteins including but not limited to GDNF or Neurturin).

For example, neuroocognitive dysfunction in, e.g., newborns or infants (e.g., 3 years old or less such as less than 3, 2.5, 2, or 1.5 years of age), preadolescent (e.g., in humans those less than 10, 9, 8, 7, 6, 5, or 4 but greater than 3 years of age), or adults, with mucopolysaccharoidosis diseases may be similarly treated. For example, besides MPS I (IDUA), MPS IIIA (Heparan-N-sulfatase; sulfaminidase), MPS IIIB (alpha-N-acetyl-glucosaminidase), MPS IIIC (Acetyl-CoA: alpha-N-acetyl-glucosaminide acetyltransferase), MPS IIID (N-acetylglucosamine 6-sulfatase), MPS VII (beta-glucoronidase), Gaucher (acid beta-glucosidase), Alpha-mannosidosis (alpha-mannosidase), Beta-mannosidosis (beta-mannosidase), Alpha-fucosidosis (alpha-fucosidase), Sialidosis (alpha-sialidase), Galactosialidosis (Cathepsin A), Aspartylglucosaminuria (aspartylglucosaminidase), GM1-gangliosidosis (beta-galactosidase), Tay-Sachs (beta-hexosaminidase subunit alpha), Sandhoff (beta-hexosaminidase subunit beta), GM2-gangliosidosis/variant AB (GM2 activator protein), Krabbe (galactocerebrosidase), Metachromatic leukodystrophy (arylsulfatase A), and other neurologic disorders including but not limited to Alzheimer's disease (expression of an antibody, such as an antibody to beta-amyloid, or an enzyme that attacks the plaques and fibrils associated with Alzheimer's), or Alzheimer's and Parkinson's diseases (expression of neuroprotective proteins including but not limited to GDNF or Neurturin), may be treated. Target gene products that may be encoded by an rAAV vector include, but are not limited to, heparan sulfate sulfatase, N-acetyl-alpha-D-glucosaminidase, beta-hexosaminidase, alpha-galactosidase, beta-galactosidase, beta-glucuronidase or glucocerebrosidase. In one embodiment, the mammal may have undergone a bone marrow transplant, e.g., HSCT, prior to administration of the rAAV. In one embodiment, the rAAV is administered to an infant (e.g., a human that is 3 years old or less such as less than 3, 2.5, 2, or 1.5 years of age), pre-adolescent (e.g., in humans those less than 10, 9, 8, 7, 6, 5, or 4 but greater than 3 years of age), or adult. In one embodiment, the rAAV is administered prior to symptom development, e.g., administered to an infant or pre-adolescent in an amount effective to prevent or inhibit one or more neurologic symptoms. In one embodiment, the rAAV is administered after symptom development, e.g., in an amount effective to inhibit or treat one or more neurologic symptoms.

Other viral vectors may be employed in the methods of the invention, e.g., viral vectors such as retrovirus, lentivirus, adenovirus, semliki forest virus or herpes simplex virus vectors.

### BRIEF DESCRIPTION OF THE FIGURES

FIGS. 1A-1E. Adeno-associated virus (AAV) vector constructs for hIDS and hSUMF1 expression. hIDS and hSUMF1 are transcriptionally regulated by the cytomegalovirus (CMV) enhancer/chicken beta-actin promoter (CB7) and by the rabbit beta globin polyadenylation signal (RBG pA), flanked with AAV2-ITRs on both 3′ and 5′ ends. For vectors co-expressing hIDS and hSUMF1, an internal ribosome entry site (IRES) is inserted in between the two open reading frames. A) AAV9 expressing hIDS (AAV9.hIDS); B) AAV9 expressing codon-optimized hIDS (AAV9.hIDSco); C) AAV9 co-expressing hIDS and human SUMF-1 (AAV9.hIDS-hSUMF1); D) AAV9 co-expressing codon-optimized hIDS and codon-optimized human SUMF-1 (AAV9.hIDSco-hSUMF1co); and E) AAV9 expressing human SUMF-1 (AAV9.hSUMF1).

FIGS. 2A-2B. Iduronate-2-sulfatase (IDS) expression after intrathecal (IT), intravenous (IV), or intracerebroventricular (ICV) administration of AAV9 IDS vectors. A) Plasma IDS activities after IT or IV administration of AAV9.hIDS. AAV9. hIDS was delivered into IDS-expressing C57BL/6 mice at 8 weeks of age via IT or IV injection (N=5 for each group). IDS activities up to 140-fold higher than wild type were observed in the plasma post IT or IV administration in IDS-expressing mice. B) IDS activities in the central nervous system (CNS) after ICV injection of different vector constructs into mucopolysaccharidosis type II (MPS II) mice. AAV9.hIDS, AAV9.hIDS-hSUMF1, and AAV9.hIDS+AAV9.hSUMF1 showed 10-40% of wild-type levels of IDS activities in most portions of the brain, while some portions showed levels comparable to wild type. The codon-optimized vector constructs did not yield efficient expression of IDS.

FIGS. 3A-3D. IDS expression and metabolic correction after ICV injection of AAV9.hIDS in MPS II mice. A) Plasma IDS activities. Plasma IDS activities were monitored every 4 weeks. B) Urine glycosaminoglycans (GAG). Urine was collected from all mice just before the animals were euthanized. The levels of urine GAG in the untreated mice were approximately two-fold higher than wild type, while the levels of urine GAG in the treated mice were normalized (p>0.05 vs. wild type). C) Average IDS activity levels in the CNS. IDS activities were assessed in all 12 regions of the brain and spinal cord of wild-type animals, untreated MPS II animals, and AAV9.hIDS-treated animals as indicated. No IDS activity was observed in the CNS of untreated MPS II mice. In AAV9.hIDS treated animals, IDS activities at 9-28% of wild type were observed in the 12 regions of the brain, except the olfactory bulb (53%) and the spinal cord (7%). D) Average IDS activity levels in the peripheral organs. The average IDS activities in each tested peripheral organ in the treated mice were 11-, 270-, 5—, and 3-fold higher than wild type (heart, liver, spleen, and kidney, respectively). Lung IDS activities were 34% of wild type. Note: One mouse in the untreated MPS II group had aberrantly high IDS activity in the lung.

FIGS. 4A-4B. AAV9.hIDS vector biodistribution after ICV injection in MPS II mice. A) Biodistribution in the CNS. Genomic DNA was extracted from the indicated tissues and IDS vector sequences quantified by real-time polymerase chain reaction. An average of 1-10 vector copies/genome equivalent (vc/ge) was observed in most area of the brain, except the right hippocampus (49 vc/ge). One mouse showing low copy numbers resulted from a failed injection. GAG accumulation data for this animal have thus been excluded in FIG. 3. B) Biodistribution in peripheral organs. An average of <1 vc/ge was detected in the heart, lung, spleen, and kidney, while an average of 60 vc/ge was detected in the liver. Two black dots on the x-axis of the right cortex and left cerebellum were wild type.

FIGS. 5A-5C. Correction of storage disease in AAV9.hIDS-treated MPS II mice. A) GAG storage in the CNS. GAG content in CNS of untreated MPS II mice was significantly higher than in wild-type and treated groups (p≤0.01). GAG content in the treated group was significantly decreased when compared to the untreated group (p<0.01). GAG content observed in the CNS of the treated mice was not significantly different from wild type (p>0.05). B) GAG content in the peripheral organs. GAG content in the tested peripheral organs of untreated MPS II mice was significantly higher than in wild-type and treated groups (p≤0.01). GAG content in the treated group was significantly decreased when compared to the untreated group (p<0.01). GAG content in the treated mice was not significantly different from the wild type in all tested peripheral organs (p>0.05). C) Prevention of hepatomegaly in AAV. hIDS treated animals. At 10 months, all mice were weighed before they were euthanized. The heart, lung, liver, spleen, and kidney were perfused, harvested, and weighed. MPS II mice showed significantly increased liver size when compared to wild-type and treated mice (p<0.001). The heart, lung, spleen, and kidney sizes showed no significant difference among all groups.

FIG. 6. Neurocognitive function assessed in the Barnes maze. Animals in all three groups were tested in the Barnes Maze. The graph depicts the average latency to escape (seconds) that each group of mice required during four trials conducted on each day for six consecutive days. The average latency to escape required for wild-type and treated groups decreased over the course of 6 days of the experiment. In contrast, there was no improvement observed for the MPS II mice from day 3 to day 6. No significant difference in the performance of treated mice versus wild-type littermates was observed, while the treated mice significantly outperformed the untreated MPS II mice on days 5 and 6 (p≤0.01). FIGS. 1-6 have data from MPSII mice treated at 2 months of age while the data in FIGS. 8-14 are from MPSII mice that were older when treated.

FIG. 7. Restored neurocognitive function in AAV9. IDS treated animals. Neurocognitive function in treated MPSII mice, untreated MPSII mice and wild-type mice. At 7 months of age the AAV9. hIDS treated animals were tested in the Barnes maze along with untreated and normal littermate control groups. After a course of 5 days of repeated testing (4 trials per day), the wild-type controls required 30 seconds to escape the platform, while the MPSII animals required 50 to 60 seconds to locate the escape. The AAV9-hIDS treated animals were significantly improved in their performance of this task (p<. 05 on day 4). This MPSII strain is neurocognitively deficient at four months of age, so these results demonstrate that treatment with AAV9-hIDS at 4 months of age restored cognitive function when the animals were subsequently tested at 7 months of age.

FIG. 8. Study design for MPSII mice with established neurological deficit. Four-month old mice were treated with AAV9.humanIDS by stereotactic injection into the right lateral ventricle. At 4 months of age MPSII animals have neurocognitive deficit in the Barnes maze. All animals were tested in the Barnes maze at 7 months of for neurologic function and euthanized at 11 months of age for biochemical analysis

FIG. 9. IDS activity in plasma in ICV treated (AAV9-hIDS) MPSII mice, untreated MPSII mice and wild-type mice. Blood samples were collected at the indicated times post-ICV infusion and assayed for IDS activity. IDS was assessed at 200 nmol/hr/mL from 4 out to 7 months, 500 times greater than the normal heterozygote level. Elevated IDS activity was detected in plasma-500x WT levels.

FIGS. 10A-10B. IDS activity in organs and tissues of treated MPSII mice, untreated MPSII mice and wild-type mice. Animals were euthanized at 11 months of age and extracts prepared from peripheral organs and from microdissected portions of the brain, as indicated. IDS assay of tissue extracts demonstrated higher than heterozygote levels of enzyme in extracts from peripheral organs (except lung), and partial restoration of normal enzyme activity in lung and in all areas of the brain.

FIGS. 11A-11B. GAG levels in organs and tissues of treated MPSII mice, untreated MPSII mice and wild-type mice. The results demonstrated reduced levels of storage material that were near normal in all areas of the brain and in peripheral tissues.

FIGS. 12A-12B. Barnes maze.

FIGS. 13A-13B. AAV9-IDS vector biodistribution after intracerebroventricular infusion at 6 months of age (post-symtpomatic). Vector copy number per genome equivalent is shown for DNA extracted from microdissected portions of the brain (A) and from peripheral tissues (B) and subjected to qPCR. DNA was extracted from the indicated tissues and AAV9-IDS vector assayed by quantitative PCR. Each dot represents the value from an individual animal, with the horizontal line indicated the mean of all samples.

FIGS. 14A-14B. qPCR data from various tissues.

FIG. 15. IDUA enzyme activity in plasma after ICV delivery of AAV-IDUA to MPSI mice (“treated”), relative to heterozygotes or control mice. Blood samples were collected on a monthly basis, and plasma was assayed for IDUA enzyme expression. Levels of IDUA were approximately 1000-fold higher than heterozygote controls. High levels of IDUA enzyme activity in plasma was observed in older MPSI mice after ICV infusion and remained higher that WT mice 6 months post-infusion. The data shows the rapid increase in plasma IDUA (i.e., 0 at 24 weeks before treatment, then 1000 to 10,000 units per mL thereafter).

FIG. 16. Improved neurocognitive function in AAV-IDUA ICV treated animals. The Barnes maze was used to assess spatial learning and memory at 10 months of age. Animals had to locate the escape hole on the maze, and were subjected to 6 trials a day for 4 days. MPS I mice displayed a significant neurocognitive deficit in locating the escape hole at compared to heterozygote controls (** P<0.001), while ICV-treated animals behaved significantly better than untreated MPS I mice (** P<0.001), and similarly to heterozygote controls.

FIG. 17. Schematic of AAV-IDUA vector for ICV delivery.

FIGS. 18A-18B. IDUA enzyme activity and neurobehavior in animals at 6 months after ICV delivery at 2 months. The results are from animals that were treated at two months and evaluated at 6 months, showing enzyme activity in the brain after the animals were sacrificed (at 6 months). The treatment prevented neurocognitive dysfunction at 6 months. Moreover, the data also show that the untreated mice have by this time developed neurocognitive dysfunction. This is the time at which the “old” mice were then treated with AAV vector (see below).

FIG. 19. Restoration of IDUA enzyme activity in brain in older MPSI mice after ICV infusion. IDUA enzyme activity in different portions of the brain, spinal cord and liver after ICV delivery of AAV-IDUA to MPSI mice (“treated”), relative to heterozygotes or control mice. Animals were sacrificed at 11 months of age, brains were microdissected and analyzed for iduronidase expression. Enzyme activities were restored to heterozygote levels in the spinal cord, and ranged from 10 to 1000-fold higher than heterozygote levels in other parts of the brain. This data is from animals treated at 6 months (post-symptomatic) then sacrificed at 11 months.

FIG. 20. GAG levels in different portions of the brain after ICV delivery of AAV-IDUA to MPSI mice (“treated”), relative to heterozygotes or control mice. Animals were sacrificed at 11 months after ICV vector infusion at 6 months, brains were microdissected and analyzed for GAG storage. GAG levels were restored to wild type or close to wild type in treated animals.

FIG. 21. Barnes maze.

FIGS. 22A-22B. A) Data showing iduronidase activities in tissues of the CNS. Activities from animals infused at 2 months are shown side by side with activities from animals infused at 6 months. B) Assessment of GAG storage in CNS tissues of animals administered AAV9-IDUA at 6 months and then sacrificed at 9 months.

### DETAILED DESCRIPTION

### Definitions

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent of the invention e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more neurological symptoms.

In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are eleven serotypes of AAVs, AAV1-AAV11, including AAV2, AAV5, AAV8, AAV9 and AAVrh10, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the invention typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

### Pathways for Delivery

There is currently no existing accepted therapy for neurologic manifestations of MPS II (Hunter syndrome). Adeno-associated virus mediated IDS gene transfer to the CNS prevents the development of neurologic dysfunction in a murine model of MPS II. Remarkably, as disclosed herein, AAV mediated IDS gene transfer to the CNS also results in the recovery of neurologic function when administered to animals that have already developed manifestations of the disease.

Despite the immense network of the cerebral vasculature, systemic delivery of therapeutics to the central nervous system (CNS) is not effective for greater than 98% of small molecules and for nearly 100% of large molecules (Partridge, 2005). The lack of effectiveness is due to the presence of the blood-brain barrier (BBB), which prevents most foreign substances, even many beneficial therapeutics, from entering the brain from the circulating blood. While certain small molecule, peptide, and protein therapeutics given systemically reach the brain parenchyma by crossing the BBB (Banks, 2008), generally high systemic doses are needed to achieve therapeutic levels, which can lead to adverse effects in the body. Therapeutics can be introduced directly into the CNS by intracerebroventricular or intraparenchymal injections.

Any route of rAAV administration may be employed so long as that route and the amount administered are prophylactically or therapeutically useful. In one example, routes of administration to the CNS include intrathecal and intracranial. Intracranial administration may be to the cisterna magna or ventricle. The term “cisterna magna” is intended to include access to the space around and below the cerebellum via the opening between the skull and the top of the spine. The term “cerebral ventricle” is intended to include the cavities in the brain that are continuous with the central canal of the spinal cord. Intracranial administration is via injection or infusion and suitable dose ranges for intracranial administration are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector delivered in about 10, 50, 100, 200, 500, 1000, or 2000 microliters. It should be understood that the aforementioned dosage is merely an exemplary dosage and those of skill in the art will understand that this dosage may be varied. Effective doses may be extrapolated from dose-responsive curves derived from in vitro or in vivo test systems.

The AAV delivered in the intrathecal methods of treatment of the present invention may be administered through any convenient route commonly used for intrathecal administration. For example, the intrathecal administration may be via a slow infusion of the formulation for about an hour. Intrathecal administration is via injection or infusion and suitable dose ranges for intrathecal administration are generally about 10³ to 10¹⁵ infectious units of viral vector per microliter delivered in, for example, 1, 2, 5, 10, 25, 50, 75or 100 or more milliliters, e.g., 1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units of viral vector.

The AAV delivered in the methods of treatment of the present invention may be administered in suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units. In one embodiment, the AAV employed for delivery is one that binds to glycans with terminal galactose residues and in one embodiment the dose is 2 to 8 fold higher than w9×10¹⁰ to less than 1×10¹¹ AAV8 genomes or infectious units of viral vector.

The therapy results in the normalization of lysosomal storage granules in the neuronal and/or meningeal tissue of the subjects as discussed above. It is contemplated that the deposition of storage granules is ameliorated from neuronal and glial tissue, thereby alleviating the developmental delay and regression seen in individuals suffering with lysosomal storage disease. Other effects of the therapy may include the normalization of lysosomal storage granules in the cerebral meninges near the arachnoid granulation, the presence of which in lysosomal storage disease result in high pressure hydrocephalus. The methods of the invention also may be used in treating spinal cord compression that results from the presence of lysosomal storage granules in the cervical meninges near the cord at C₁-C₅ or elsewhere in the spinal cord. The methods of the invention also are directed to the treatment of cysts that are caused by the perivascular storage of lysosomal storage granules around the vessels of the brain. In other embodiments, the therapy also may advantageously result in normalization of liver volume and urinary glycosaminoglycan excretion, reduction in spleen size and apnea/hypopnea events, increase in height and growth velocity in prepubertal subjects, increase in shoulder flexion and elbow and knee extension, and reduction in tricuspid regurgitation or pulmonic regurgitation.

The intrathecal administration of the present invention may comprise introducing the composition into the lumbar area. Any such administration may be via a bolus injection. Depending on the severity of the symptoms and the responsiveness of the subject to the therapy, the bolus injection may be administered once per week, once per month, once every 6 months or annually. In other embodiments, the intrathecal administration is achieved by use of an infusion pump. Those of skill in the art are aware of devices that may be used to effect intrathecal administration of a composition. The composition may be intrathecally given, for example, by a single injection, or continuous infusion. It should be understood that the dosage treatment may be in the form of a single dose administration or multiple doses.

As used herein, the term “intrathecal administration” is intended to include delivering a pharmaceutical composition directly into the cerebrospinal fluid of a subject, by techniques including lateral cerebroventricular injection through a burrhole or cistemal or lumbar puncture or the like. The term “lumbar region” is intended to include the area between the third and fourth lumbar (lower back) vertebrae and, more inclusively, the L2-S1 region of the spine.

Administration of a composition in accordance with the present invention to any of the above mentioned sites can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment of the invention, the rAAV is administered by lateral cerebroventricular injection into the brain of a subject. The injection can be made, for example, through a burr hole made in the subject's skull. In another embodiment, the enzyme and/or other pharmaceutical formulation is administered through a surgically inserted shunt into the cerebral ventricle of a subject. For example, the injection can be made into the lateral ventricles, which are larger, even though injection into the third and fourth smaller ventricles can also be made. In yet another embodiment, the compositions used in the present invention are administered by injection into the cisterna magna or lumbar area of a subject.

In one embodiment, an immune suppressant or immunotolerizing agent may be administered by any route including parenterally. In one embodiment, the immune suppressant or immunotolerizing agent may be administered by subcutaneous, intramuscular, or intravenous injection, orally, intrathecally, or intracranially, or by sustained release, e.g., using a subcutaneous implant. The immune suppressant or immunotolerizing agent may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, compositions may comprise an aqueous solution of a water soluble pharmaceutically acceptable salt of the active acids according to the invention, desirably in a concentration of 0.01-10%, and optionally also a stabilizing agent and/or buffer substances in aqueous solution. Dosage units of the solution may advantageously be enclosed in ampules.

The composition, e.g., rAAV containing composition, immune suppressant containing composition or immunotolerizing composition, may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the immune suppressant or immunotolerizing agent is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the immune suppressant or immunotolerizing agent containing composition is administered may vary within a wide range and will depend on various factors such as the severity of the disease, the age of the patient, etc., and may have to be individually adjusted. A possible range for the amount which may be administered per day is about 0.1 mg to about 2000 mg or about 1 mg to about 2000 mg. The compositions containing the immune suppressant or immunotolerizing agent may suitably be formulated so that they provide doses within these ranges, either as single dosage units or as multiple dosage units. In addition to containing an immune suppressant, the subject formulations may contain one or more rAAV encoding a therapeutic gene product.

Compositions described herein may be employed in combination with another medicament. The compositions can appear in conventional forms, for example, aerosols, solutions, suspensions, or topical applications, or in lyophilized form.

Typical compositions include a rAAV, and optionally an immune suppressant, a permeation enhancer, or a combination thereof, and a pharmaceutically acceptable excipient which can be a carrier or a diluent. For example, the active agent(s) may be mixed with a carrier, or diluted by a carrier, or enclosed within a carrier. When the active agent is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active agent(s). Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The agent(s) may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present invention may include, for example, micelles or liposomes, or some other encapsulated form, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few um.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swallen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition of the invention can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens. For example, in addition to solubility, efficient delivery to the CNS following administration may be dependent on membrane permeability.

Generally, the active agents are dispensed in unit dosage form including the active ingredient together with a pharmaceutically acceptable carrier per unit dosage. Usually, dosage forms suitable for administration include from about 125 μg to about 125 mg, e.g., from about 250 μg to about 50 mg, or from about 2.5 mg to about 25 mg, of the compounds admixed with a pharmaceutically acceptable carrier or diluent.

Dosage forms can be administered daily, or more than once a day, such as twice or thrice daily. Alternatively, dosage forms can be administered less frequently than daily, such as every other day, or weekly, if found to be advisable by a prescribing physician.

The invention will be described by the following non-limiting examples.

### Example I

### AAV Vector-Mediated Iduronidase Gene Delivery in a Murine Model of Mucopolysaccharidosis Type I: Comparing Different Routes of Delivery to the CNS

Mucopolysaccharidosis type I (MPS I) is an inherited metabolic disorder caused by deficiency of the lysosomal enzyme alpha-L-iduronidase (IDUA). Systemic and abnormal accumulation of glycosaminoglycans is associated with growth delay, organomegaly, skeletal dysplasia, and cardiopulmonary disease. Individuals with the most severe form of the disease (Hurler syndrome) suffer from neurodegeneration, mental retardation, and early death. The two current treatments for MPS I (hematopoietic stem cell transplantation and enzyme replacement therapy) cannot effectively treat all central nervous system (CNS) manifestations of the disease.

With respect to gene therapy, it was previously demonstrated that intravascular delivery of AAV9 in adult mice does not achieve widespread direct neuronal targeting (see Foust et al, 2009). Previous work also showed that direct injection of AAV8-IDUA into the CNS of adult IDUA-deficient mice resulted in a low frequency or a poor level of transgene expression. The following examples, which use a pre-clinical model for the treatment of MPS1, surprisingly demonstrate that direct injection of AAV9-IDUA into the CNS of immunocompetent adult IDUA-deficient mice resulted in IDUA enzyme expression and activity that is the same or higher than IDUA enzyme expression and activity in wild-type adult mice.

### Methods

AAV9-IDUA preparation. The AAV-IDUA vector construct (MCI) has been previously described (Wolf et al., 2011) (mCags promoter). AAV-IDUA plasmid DNA was packaged into AAV9 virions at the University of Florida Vector Core, yielding a titer of 3×10¹³ vector genomes per milliliter.

ICV infusions. Adult Idua-/-mice were anesthetized using a cocktail of ketamine and xylazine (100 mg ketamine+10 mg xylazine per kg) and placed on a stereotactic frame. Ten microliters of AAV9-IDUA were infused into the right-side lateral ventricle (stereotactic coordinates AP 0.4, ML 0.8, DV 2.4 mm from bregma) using a Hamilton syringe. The animals were returned to their cages on heating pads for recovery.

Intrathecal infusions. Infusions into young adult mice were carried out by injection of 10 μL AAV vector containing solution between the L5 and L6 vertebrae 20 minutes after intravenous injection of 0.2 mL 25% mannitol.

Immunotolerization. Newborn IDUA deficient mice were injected through the facial temporal vein with 5 L containing 5.8 μg of recombinant iduronidase protein (Aldurazyme), and then the animals were returned to their cage.

Cyclophosphamide immunosuppression. For immunosuppression, animals were administered cyclophosphamide once per week at a dose of 120 mg/kg starting one day after infusion with AAV9-IDUA vector.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C. Samples were thawed and homogenized in 1 mL of PBS using a motorized pestle and permeabilized with 0.1% Triton X-100. IDUA activity was determined by fluorometric assay using 4MU-iduronide as the substrate. Activity is expressed in units (percent substrate converted to product per minute) per mg protein as determined by Bradford assay (BioRad).

Tissues. Tissue homogenates were clarified by centrifugation for 3 minutes at 13,000 rpm using an Eppendorf tabletop centrifuge model 5415D (Eppendorf) and incubated overnight with proteinase K, DNase1, and Rnase. GAG concentration was determined using the Blyscan Sulfated Glycosaminoglycan Assay (Accurate Chemical) according to the manufacturer's instructions.

### Results

Iduronidase-deficient mice were administered AAV either intracerebroventricularly (ICV) or intrathecally (IT). To prevent immune response, animals were either immunosuppressed with cyclophosphamide (CP), immunotolerized at birth by intravenous administration of human iduonidase protein (aldurazyme), or the injections were carried out in NOD-SCID immunodeficient mice that were also iduronidase deficient. Animals were sacrificed at the indicated time post-treatment, the brains were microdissected and extracts assayed for iduronidase activity.

Immunodeficient, IDUA deficient animals that were injected ICV with AAV-IDUA vector exhibited high levels of IDUA expression (10 to 100 times wild type) in all areas of the brain, with the highest level observed in the brain stem and thalamus (“rest”).

Immunosuppressed animals administered AAV vector by ICV route had a relatively lower level of enzyme in the brain compared to the immunodeficent animals. Note that immunosuppression may have been compromised in these animals because CP was withdrawn 2 weeks before sacrifice due to poor health.

Immunosuppressed animals were administered AAV vector by the IT route. Immunotolerized animals administered AAV vector ICV exhibited widespread IDUA activity in all parts of the brain, similar to that observed in the immunodeficient animals, indicating the effectiveness of the immunotolerization procedure.

GAG storage material was assayed in the different sections of the brain for all four of the test groups. For each group, the mean of each portion of the brain is shown on the left, the values for each of the individual animals is shown on the right. IDUA deficient animals (far left) contained high levels of GAG compared to wild type animals (magenta bar). GAG levels were at wild-type or lower than wild type for all portions of the brain in all groups of AAV-treated animals. GAG levels were slightly although not significantly higher than wild-type in cortex and brainstem of animals administered AAV9-IDUA intrathecally.

### Conclusions

The results show high and widespread distribution of IDUA in the brain regardless of the route of delivery (ICV or IT) although IDUA expression in striatum and hippocampus was lower in animals injected IT versus ICV. There appears to be an immune response since immune deficient mice have higher levels of expression than immunocompetent mice. With regard to ICV injection, when CP was withdrawn early, IDUA expression is lower. In addition, immunotolerization was effective in restoring high levels of enzyme activity. Further, GAG levels were restored to normal in all treated experimental groups of mice.

### Example II

### Methods

AAV9-IDUA Preparation. AAV-IDUA plasmid was packaged into AAV9 virions at either the University of Florida vector core, or the University of Pennsylvania vector core, yielding a titer of 1-3×10¹³ vector genomes per milliliter.

ICV infusions. See Example I.

Intrathecal infusions. See Example I.

Immunotolerization. As in Example 1 except: for multiple tolerizations, newborn IDUA deficient mice were injected with the first dose of Aldurazyme in the facial temporal vein, followed by 6 weekly injections administered intraperitoneally.

Cyclophosphamide immunosuppression. See Example I.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C.

Tissue IDUA activity. Tissue samples were thawed and homogenized in saline in a tissue homogenizer. Tissue homogenates were clarified by centrifugation at 15,000 rpm in a benchtop Eppendorf centrifuge at 4° C. for 15 minutes. Tissue lysates (supernatant) were collected and analyzed for IDUA activity and GAG storage levels.

Tissue GAG levels. Tissue lysates were incubated overnight with Proteinase K, RNase and DNase. GAG levels were analyzed using the Blyscan Sulfated Glycosaminoglycan Assay according to the manufacturer's instructions.

IDUA Vector copies. Tissue homogenates were used for DNA isolation and subsequent QPCR, as described in Wolf et al. (2011).

### Results

Animals were administered AAV9-IDUA vector either by intracerebroventricular (ICV) or intrathecal (IT) infusion. Vector administration was carried out in NOD-SCID immunodeficient (ID) mice that were also IDUA deficient, or in IDUA deficient mice that were either immunosuppressed with cyclophosphamide (CP), or immunotolerized at birth by a single or multiple injections of human iduronidase protein (Aldurazyme). All vector administrations were carried out in adult animals ranging in age from 3-4.5 months. Animals were injected with 10 μL of vector at a dose of 3×10¹¹ vector genomes per 10 microliters.

IDUA enzyme activities in intracranially infused, immunodeficient, IDUA deficient mice were high in all areas of the brain, ranging from 30- to 300-fold higher than wild type levels. Highest enzyme expressions were seen in thalamus and brain stem, and in the hippocampus.

Animals that were injected intracranially and immunosuppressed with cyclophosphamide (CP) demonstrated significantly lower levels of enzyme activity than other groups. However, CP administration in this case had to be withdrawn 2 weeks prior to sacrifice due to poor health of the animals.

IDUA enzyme levels in animals tolerized at birth with IDUA protein (Aldurazyme) and administered vector intracranially were high in all parts of the brain that ranged from 10- to 1000-fold higher than wild type levels, similar to levels achieved in immunodeficient animals, indicating the effectiveness of the immunotolerization procedure.

IDUA enzyme levels in mice that were injected intrathecally and administered CP on a weekly basis were elevated and were observed in all parts of the brain, especially in the cerebellum and the spinal cord. Levels of enzyme were the lowest in the striatum and hippocampus with activities at wild type levels.

IDUA deficient mice were tolerized with Aldurazyme as described, and injected with vector intrathecally. There was widespread IDUA enzyme activity in all parts of the brain, with highest levels of activity in the brain stem and thalamus, olfactory bulb, spinal cord and the cerebellum. Similarly, the lowest levels of enzyme activity were seen in the striatum, cortex and hippocampus.

Control immunocompetent IDUA deficient animals were infused with vector intrathecally, without immunosuppression or immunotolerization. The results indicate that although enzyme activities were at wild type levels or slightly higher, they are significantly lower than what was observed in animals that underwent immunomodulation. The decreases in enzyme levels were especially significant in the cerebellum, olfactory bulb and thalamus and brain stem, areas that expressed the highest levels of enzyme in immunomodulated animals.

Animals were assayed for GAG storage material. All groups demonstrated clearance of GAG storage, with GAG levels similar to that observed in wild type animals. Animals that were immunosuppressed and injected with AAV9-IDUA vector intrathecally had GAG levels in the cortex that were slightly higher than wild type, but still much lower than untreated IDUA deficient mice.

The presence of AAV9-IDUA vector in animals that were immunotolerized and injected with vector either intracranially or intrathecally was evaluated by QPCR. IDUA copies per cell were higher in animals infused intracranially in comparison with animals infused intrathecally, which is consistent with the higher level of enzyme activity seen in animals injected intracranially.

### Conclusions

High, widespread, and therapeutic levels of IDUA were observed in all areas of the brain after intracerebroventricular and intrathecal routes of AAV9-IDUA administration in adult mice. Enzyme activities were restored to wild type levels or slightly higher in immunocompetent IDUA deficient animals infused with AAV-IDUA intrathecally. Significantly higher levels of IDUA enzyme were observed for both routes of vector injection in animals immunotolerized starting at birth by administration of IDUA protein.

### Example III

Mucopolysaccharidosis type II (MPS II; Hunter Syndrome) is an X-linked recessive inherited lysosomal storage disease caused by deficiency of iduronate-2-sulfatase (IDS) and subsequent accumulation of glycosaminoglycans (GAGs) dermatan and heparan sulphate. Affected individuals exhibit a range in severity of manifestations physically, neurologically, and shortened life expectancy. For example, affected individuals exhibit a range in severity of manifestations such as organomegaly, skeletal dysplasias, cardiopulmonary obstruction, neurocognitive deficit, and shortened life expectancy. There is no cure for MPS II at the moment. Current standard of care is enzyme replacement therapy (ELAPSRASE; idursulfase), which is used to manage disease progression. However, enzyme replacement therapy (ERT) does not result in neurologic improvement. As hematopoetic stem cell transplantation (HSCT) has not shown neurologic benefit for MPS II, there is currently no clinical recourse for patients exhibiting neurologic manifestations of this disease, and new therapies are desperately needed.

AAV9 vectors are developed for delivery of the human IDS coding sequence (AAV9-hIDS) into the central nervous system of MPS II mice to restore IDS levels in the brain and prevent the emergence of neurocognitive deficits in the treated animals. In particular, a series of vectors were generated that encode human IDS with or without the human sulfatase modifying factor-1 (SUMF-1), required for activation of the sulfatase active site. Three routes of administration were used in these experiments: Intrathecal (IT). Intracerebroventricular (ICV) and Intravenous (IV). No significant difference in the enzyme level was found between mice that were treated with AAV9 vector transducing hIDS alone and mice that were treated with AAV9 vector encoding human IDS and SUMF-1, regardless of the route of administration. IT-administrated NOD.SCID (IDS Y+) and C57BL/6 (IDS Y+) did not show elevated IDS activity in the brain and spinal cord when compared to untreated animals, while plasma showed ten-fold higher (NOD.SCID) and 150-fold higher (C57BL/6) levels than untreated animals. IDS-deficient mice intravenously administered AAV9-hIDS exhibited IDS activities in all organs that were comparable to wild type. Moreover, the plasma of IV injected animals showed enzyme activity that was 100-fold higher than wild type. IDS-deficient mice administered AAV9-hIDUA ICV showed IDS activities comparable to wild type in most areas of the brain and peripheral tissues, while some portions of the brain showed two-to four-fold higher activity than wild type. Furthermore, IDS activity in plasma was 200-fold higher than wild type. Surprisingly, IDS enzyme activity in the plasma of all treated animals showed persistence for at least 12 weeks post injection; therefore, IDS enzyme was not immunogenic at least on the C57BL/6 murine background. Additional neurobehavioral testing was conducted using the Barnes maze to differentiate neurocognitive deficits of untreated MPS II animals from that of wild type littermates. It was found that the learning capability of affected animals is distinctively slower than that observed in littermates. Thus, Barnes maze is used to address the benefit of these therapies in the MPS II murine model. These results indicate potential of therapeutic benefit of AAV9 mediated human IDS gene transfer to the CNS to prevent neurologic deficiency in MPS II.

In summary, intracerebroventricular (ICV) injection of AAV9-hIDS resulted in systemic correction of IDS enzyme deficiency, including wild-type levels of IDS in the brain. Co-delivery of hIDS with hSUMF-1 did not increase IDS activity in tissues. hIDS expression was non-immunogenic in WT and MPS II C57BL/6 mice.

The following provides further details in this regard.

Mucopolysaccharidosis type II (MPS II, Hunter syndrome) is a rare x-linked recessive lysosomal disorder caused by defective Iduronate-2-sulfatase (IDS) resulting in accumulation of heparan sulfate and dermatan sulfate glycosaminoglycans (GAGs). Enzyme replacement is the only FDA-approved therapy available for MPS II, but it is expensive and does not improve neurologic outcomes in MPS II patients. As described below, this study evaluated the effectiveness of IDS-encoding adeno-associated virus (AAV) vector encoding human IDS delivered intracerebroventricularly in a murine model of MPS II. Supraphysiological levels of IDS were observed in the circulation (160-fold higher than wild type) for at least 28 weeks post-injection and in most tested peripheral organs (up to 270-fold) at 10 months postinjection. In contrast, only low levels of IDS were observed (7% to 40% of wild type) in all areas of the brain. Sustained IDS expression had a profound effect on normalization of GAG in all tested tissues and on prevention of hepatomegaly. Additionally, sustained IDS expression in the CNS had a prominent effect in preventing neurocognitive deficit in MPS II mice treated at two months of age. The present study demonstrates that CNS-directed, AAV9 mediated gene transfer is a potentially effective treatment for Hunter syndrome as well as other monogenic disorders with neurologic involvement.

### INTRODUCTION

The mucopolysaccharidoses (MPSs) are a group of lysosomal disorders caused by deficiency of any one of 11 lysosomal hydrolase that catalyze the breakdown of glycosaminoglycans (GAGs). MPS type II (MPS II; Hunter syndrome), is an X-linked recessive caused by deficiency of iduronate-2-sulfatase (IDS) with subsequent accumulation of substrate (GAGs) in tissues of affected individuals associated with hepatosplenomegaly, skeletal dysplasia, joint stiffness, and airway obstruction. In severe cases, affected individuals exhibit neurocognitive deficits and succumb to the illness in adolescence. The current and only treatment available for MPS II is enzyme replacement therapy (ERT), which is used to mitigate disease progression but without neurologic improvement. Hematopoietic stem cell transplantation, which has been shown to provide long-term benefits for MPS I (Whitley et al., 1993), has not been reported to ameliorate neurodegenerative disease in severe cases of MPS II (McKinnis et al. 1996; Vellodi et al. 2015; Hoogerbrugge et al. 1995).

The Sleeping Beauty (SB) transposon system and minicircles are two non-viral gene therapy platforms that have been successfully used in mice for systemic diseases such as MPS type I and type VII (Aronovich et al. 2009; Aronovich et al. 2007; Osborn et al. 2011). Despite being efficient and providing sustained expression in vivo (Aronovich et al. 2007; Chen 2003), the major drawback of these systems is the inability to penetrate the BBB (Aronovich and Hackett 2015) which has not yet been resolved. This limits the effectiveness of non-viral gene therapy systems for the CNS.

Various viral vectors have been extensively studied in gene therapy clinical trials for many diseases because their potency and sustained expression (Kaufmann et al. 2013). Amongst these vehicles, adeno-associated viral vectors (AAVs) have been shown to be promising candidates for clinical trials in mediating gene transfer for monogenic disorders (Tanaka et al. 2012; Bennett et al. 2012; Nathwani et al. 2014). Unlike other AAV serotypes, adeno-associated viral vector serotype 9 (AAV9) has been demonstrated in many animal models to not only efficiently transduce the CNS and peripheral nervous tissues (PNS), but also penetrate the BBB and transduce various cell types in peripheral tissues (Duque et al., 2009; Foust et al., 2009; Huda et al., 2014; Schuster et al., 2014). Thus AA9 outperforms other viral vectors as a candidate for systemic correction including CNS for monogenic disorders such as MPS II. Herein is reported the effectiveness of CNS-directed, AAV9 mediated human IDS gene transfer to correct IDS deficiency and prevent neurocognitive impairment in a murine model of MPS II.

### Materials and Methods

### AAV Vector Assembly and Packaging

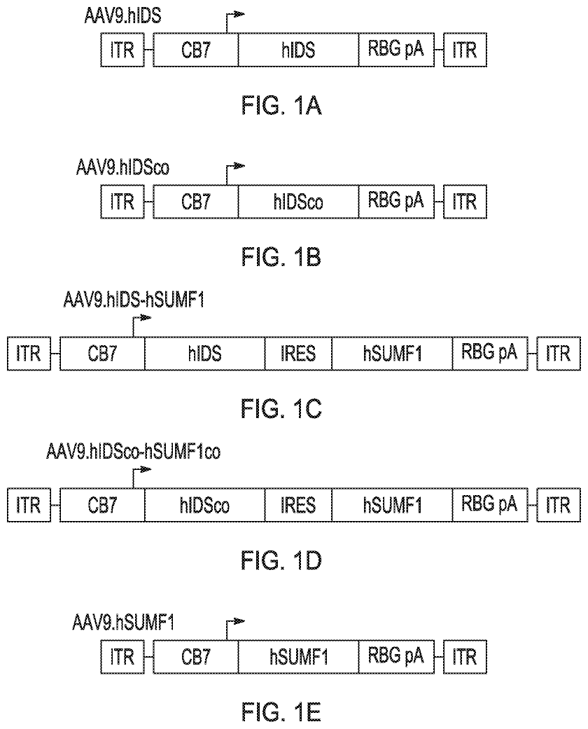

All vectors were constructed, packaged, and purified at the Penn Vector Core (Philadelphia, PA) and were provided by REGENXBIO, Inc. (Rockville, MD). In brief, the expression cassettes contained a chicken beta-actin (CB7) promoter with cytomegalovirus (CMV) enhancer followed by hIDS or human sulfatase modifying factor 1 (hSUMF1), rabbit beta-actin polyadenylation signal on the backbone of AAV2 inverted terminal repeats (ITR) on both 3′- and 5′-ends. Co-expression constructs included an internal ribosome entry site (IRES) positioned between hIDS and SUMF1 to initiate translation of SUMF1 downstreamof the IRES. In this study we investigated five different vector constructs: AAV9 expressing human IDS alone (AAV9.hIDS; FIG. 1A); AAV9 expressing codonoptimized human IDS (AAV9.hIDSco; FIG. 1B); AAV9 co-expressing human IDS and human SUMF1 (AAV9.hIDS-hSUMF1; FIG. 1C); AAV9 co-expressing codon-optimized human IDS and codon-optimized human SUMF1 (AAV9.hIDScohSUMF1co; FIG. 1D); and AAV9 expressing human SUMF1 alone (AAV9.hSUMF1; FIG. 1E). AAV vectors were packaged by co-transfecting three plasmids-AAV cis (Fi. 1), AAV trans (pAAV2/9 rep and cap), and adenovirus helper (pAdDF6)-into HEK 293 cells (Lock et al., 2010). AAV vector was then purified from supernatants using a Profile II depth filter and concentrated by tangential flow filtration (TFF). The concentrated feed stock was reclarified by iodixanol gradient centrifugation and then re-concentrated using a TFF cassette with a 100 kDa MWCO HyStream screen channel membrane. The purified vector was then tested for purity by SDS-PAGE and for potency by quantitative polymerase chain reaction (qPCR) (Lock et al., 2010).

### Animal Care and Husbandry

All animal care and experimental procedures were conducted under approval of the Institutional Animal Care and use Committee (IACUC) of the University of Minnesota. NOD.SCID mice were purchased from The Jackson Laboratory and C57BL/6 wild-type mice were purchased from National Cancer Institute. C57BL/6 iduronate-2-sulphatase knockout (IDS KO) mice were kindly provided by Dr. Joseph Muenzer (University of North Carolina, NC) and maintained under specific pathogen-free conditions at the Research Animal Resources (RAR) facilities of the University of Minnesota. MPS II male pups (IDS^{-/0}) were generated by breeding heterozygous (IDS^{+/-}) females to wild type (IDS+/0) C57BL/6 males. All pups were genotyped by PCR.

### AAV Vector Administration

For intrathecal (IT) injections, eight-week-old mice were injected with a dose of 5.6×10¹⁰ vector copies (vc) of AAV9 vector between the L5 and L6 vertebrae, as previously described (Vulchanova et al., 2010). The injection was performed in conscious animals in a 10-15 second duration. For intravenous (IV) injections, animals were briefly restrained and injected via the tail-vein with a dose of 5.6×10¹⁰ vc. Intracerebroventricular (ICV) injections were carried out in adult 8-week-old mice, as previously described (Janson et al., 2014). Briefly, animals were injected intraperitoneally with a ketamine/xylazine mixture (100 mg/kg ketamine, 10 mg/kg xylazine) to produce deep anesthesia and then mounted in a stereotactic frame (Kopf Model 900). An incision was made to expose the cranium, a small hole was drilled as a site for the injection, and then a Hamilton syringe (Model 701) was used to carry out the infusion at a rate approximately 0.5 IL/minute by hand. The syringe was left in place for an additional 3 min and then slowly withdrawn over a period of at least 2 minutes. The scalp was sutured after completion of the injection, and after recovery from the anesthesia, the mouse was returned to standard housing. All of the mice received a 3-day course of Ketoprofen (2.5-5.0 mg/kg) subcutaneously and Baytril 5 mg/kg intraperitoneally to prevent infection and inflammation post surgery.

### Sample Collection and Preparation

Blood was collected by submandibular puncture using sterile 5 mm lancets (Goldenrode™) into Microvette® heparinized coated tubes (Sarstedt AG & Co.) and centrifuged in an Eppendorf centrifuge 5415D at 7,000 rpm for 10 min. Plasma was collected and stored at −20° C. to −80° C. for IDS assay. Urine was collected and stored at −20° C. until used for creatinine and GAG assay. Organs were harvested by first determining animal weight using an OHAUS® CS 200 scale before euthanasia using a CO₂ fume chamber at 2 L/minutes for 3 minutes. The animals were perfused with 60 mL of 1× phosphate-buffered saline (PBS) in a 60 ml syringe (BD) with a SURFLO® winged infusion set (TERUMO®) size 23G·x 3/4 “by hand pressure. The heart, lung, liver, spleen, kidney, and spinal cord were harvested and weighed using a Sartorius BP 61S scale. The brain was micro-dissected into left and right cerebellum, cortex, hippocampus, striatum, olfactory bulb, and thalamus/brainstem. The organs were immediately snap frozen and stored at −70° C. until further tissue processing.

For tissue processing, the cerebellum, hippocampus, striatum, and olfactory bulb were added into preassigned 1.5 mL locked-cap microtubes (Eppendorf) containing one scoop (0.2 g/scoop) of 0.5 mm glass beads (Next Advance) in 250 I L of sterile saline solution. The thalamus/brainstem, cortex, and spinal cord were added into assigned locked-cap microtubes containing two scoops of 0.5 mm glass beads in 400 IL of sterile saline solution. Half of the lung and the whole spleen were added into assigned tubes containing two scoops of 0.9-2.0 mm stainless steel blend (0.6 g/scoop) in 400 I L of saline solution. The heart, about 0.3 g of liver, and one kidney were added into assigned tubes containing three scoops (a mixture of two scoops of 0.9-2.0 mm stainless steel blend [0.6 g/scoop] and one scoop of 3.2 mm stainless steel beads [0.7 g/scoop]) in 600 I L of sterile saline solution. All of the prepared samples in the bead tubes were then homogenized using a Bullet blender® STORM bead mill homogenizer (Next Advance) at speed 12 for 5 minutes to generate tissue homogenate. Fifty microliters of tissue homogenates were transferred into 1.5 mL microtubes (GeneMate) and stored at −20° C. to −80° C. for qPCR. The remaining tissue homogenates were clarified using an Eppendorf centrifuge 5424R at 13,000 rpm for 15 minutes at 4° C. All of the supernatants (tissue lysates) were transferred into new microtubes and stored at −20° C. to −80° C. until used for IDS, GAG, and protein assays.

### Iduronate Sulfatase Assay

IDS enzyme activity was measured in tissue lysates using 4-methylumbelliferyl-α-L-iduronide-2-sulphate disodium (4-MU-αldoA-2S; Toronto Research Chemical Incorporation; cat. #M334715) as substrate in a two-step assay. Tissue lysates were mixed with 1.25 mM MU-αldoA-2S (in 0.1 M sodium acetate buffer pH 5.0+10 mM lead acetate+0.02% sodium azide) and incubated at 37° C. for 1.5 hours. The first-step reaction was terminated with PiCi buffer to stop IDS enzyme activity (0.2 M Na₂HPO₄/0.1 M citric-acid buffer, pH 4.5+0.02% Na-azide). A final concentration of 1 g/mL Iduronidase (IDUA; R&D Systems; cat. #4119-GH-010) was added into the tubes to start the second-step reaction. The tubes were incubated overnight at 37° C. to cleave 4-MU-IdoA into 4-MU. The secondstep reaction was terminated by adding 200 μL of stop buffer (0.5M Na₂CO_{3+0.5} M NaHCO₃, 0.025% Triton X-100, pH 10.7). The tubes were centrifuged using an Eppendorf centrifuge 5415D at 13,000 rpm for 1 minute. Supernatants were transferred into a round-bottom black 96-well plate and fluorescence measured at excitation 365 nm and emission 450 nm, 75 sensitivity using a Synergy MX plate reader and spectrophotometer (Bio Tek) with Gen5 plate reader program. Enzyme activity is expressed in nmol/h/mL plasma for plasma samples and in nmol/h/mg protein for tissue extracts. Protein was determined using the Pierce™ 660 nm Protein Assay Reagent with bovine serum albumin as a standard (cat. #23208; Thermo Scientific).

### Glycosaminoglycan Assay

Tissue lysates were incubated overnight with Proteinase K, DNase1, and RNase, as previously described (Wolf et al., 2011), then, GAG contents were assessed using the Blyscan™ Sulfated Glycosaminoglycan Assay kit (Biocolor Life Science Assays; Accurate Chemical). Blyscan glycosaminoglycan standard 100 μg/mL (cat. #CLRB 1010; Accurate Chemical) was used to make a daily standard curve. Absorbance was measured at 656 nm using a Synergy MX plate reader and spectrophotometer (Bio Tek) with the Gen5 plate reader program. The blank value was subtracted from all readouts. Tissue GAG content is reported in micrograms GAG per milligrams protein, and urine GAG content is reported as micrograms GAG per milligrams creatinine. Urine creatinine was measured using the Creatinine Assay Kit (Sigma-Aldrich®) according to the manufacturer's instructions.

qPCR for IDS Vector Sequences

Tissue homogenates were mixed with 300 μL cell lysis buffer (5 Prime) and with 100 μg proteinase K, gently rocking overnight at 55° C. DNA was isolated from the sample by phenol/chloroform extraction. Reaction mixtures of 20 μl contained 60 ng of DNA template, 10 μL of FastStart Taqman Probe Master mix (Roche), 200 nM each of forward and reverse primers, and 100 nM of Probe36 (#04687949001; Roche). A C1000 Touch™ Thermo Cycler (Bio-Rad) equipped with CFX manager software v3.1 was used for qPCR reaction. The PCR conditions were: 95° C. for 10 minutes, followed by 40 cycles of 95° C. for 15 seconds and 60° C. for 1 minute. IDS primers used were: forward primer-5′-TCCCTTACCTCGACCCTTTT-3′ (SEQ ID NO: 4); IDS reverse primer-5′-CACAAGGTCCATGGATTGC-3′ (SEQ ID NO: 5). To prepare the standard, pENN.AAV.CB7.hIDS was linearized by digestion with Sall restriction enzyme (New England BioLab, Inc.). The linearized plasmid DNA was then purified using the 5Prime DNA Extraction kit. The plasmid DNA concentration was measured using a NanoDrop 1000 spectrophotometer (Thermo Scientific) with the NanoDrop 1000 3.7.0 program. The purified linearized plasmid DNA was then diluted to prepare the qPCR standard curve. UltraPure™ distilled water (Invitrogen) was used as negative control.A10-fold dilution series of linearized plasmid was used to generate a standard curve with a range of 1-10⁸ plasmid copies per assay in duplicate with amplification efficiencies between 90% and 110% and R² of 0.96-0.98. Vector copy was calculated based on a daily standard curve and expressed as vector copies per cellular genome equivalent (vc/ge).

### Neurocognitive Testing in the Barnes Maze

The Barnes maze (Barnes, 1979) is a circular platform measuring approximately 4 feet in diameter and is elevated approximately 4 feet from the floor with 40 holes spaced equally around the perimeter. All of the holes are blocked except for only one hole that is pen for the mouse to escape the platform. Different visual cues were attached to each of the four walls for the mouse to use as spatial navigators. At 6 months of age, test mice were placed in the middle of the platform with an opaque funnel covering the mouse. The cover was lifted, releasing the mouse and exposing it to bright light. The animal is expected to complete the task by escaping the platform using the one open hole within 3 minutes. Each mouse was subjected to four trials per day for a total of 6 days. The time that the mouse required to escape the platform in each trial was recorded, and the average was calculated for each day in each group.

### Statistical Analysis

Data are reported as mean-standard error (SE). Statistical analyses were performed using Prism 6. Two-way analysis of variance with Tukey's post test was used to evaluate the significance of differences among test groups for IDS assay, GAG assay, and neurobehavioral assay, with a p-value of <0.05 considered significant. A two-tailed t-test on Microsoft Excel was used to evaluate differences in IDS activities between the left and the right hemispheres of the microdissected brain.

### Results

### Pilot Studies: Comparison of Vector Constructs and Route of Administration to Achieve IDS Expression in the CNS

A pilot study was conducted to compare several AAV vector constructs and to find a suitable route of administration resulting in IDS expression in the CNS. NOD.SCID mice were used for this study to circumvent the potential complication of an anti-IDS immune response. The four vectors shown in FIG. 1A-D (AAV9.hIDS, AAV9.hIDSco, AAV9.hIDS-hSUMF1, and AAV9.hIDSco-hSUMF1co) were delivered by IT administration, as described in the Materials and Methods. SUMF1 encodes an enzyme which post-translationally oxidizes an active site cysteine in lysosomal sulfatases, including IDS, converting the enzyme into a catalytically active form (Sabourdy et al., 2015). The addition of SUMF1 to some of the vectors was to determine if SUMF1 activity is rate-limiting in producing active IDS protein when IDS is overexpressed. Five untreated IDS+NOD. SCID mice were used as a control group. Six weeks post injection, the mice were euthanized, and the brain was microdissected into different portions. Our parallel studies in MPS I have demonstrated supraphysiological activities of IDUA in the CNS post-IT injection of AAV9 vector encoding hIDUA (Belur et al., 2014). We thus expected to see high levels of IDS, exceeding the endogenous level in IDS+NOD.SCID mice administered AAV9.hIDS vector. Surprisingly, we observed no significant increase of IDS activity in the CNS exceeding the endogenous level of uninjected NOD.SCID mice, regardless of vector construct (data not shown). AAV9.hIDSwas also injected into two groups of three wild type C57BL/6 (IDS+C57BL/6) mice at 8 weeks of age, one group via IT administration and the other group via IV administration. Again, no significant increase in the level of IDS activity in the CNS above the endogenous level of untreated controls was observed (data not shown). Thus, neither IT nor IV injection of IDS-encoding AAV vector appeared to be a suitable route of administration.

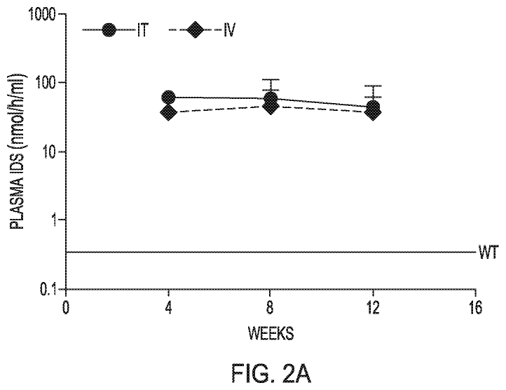

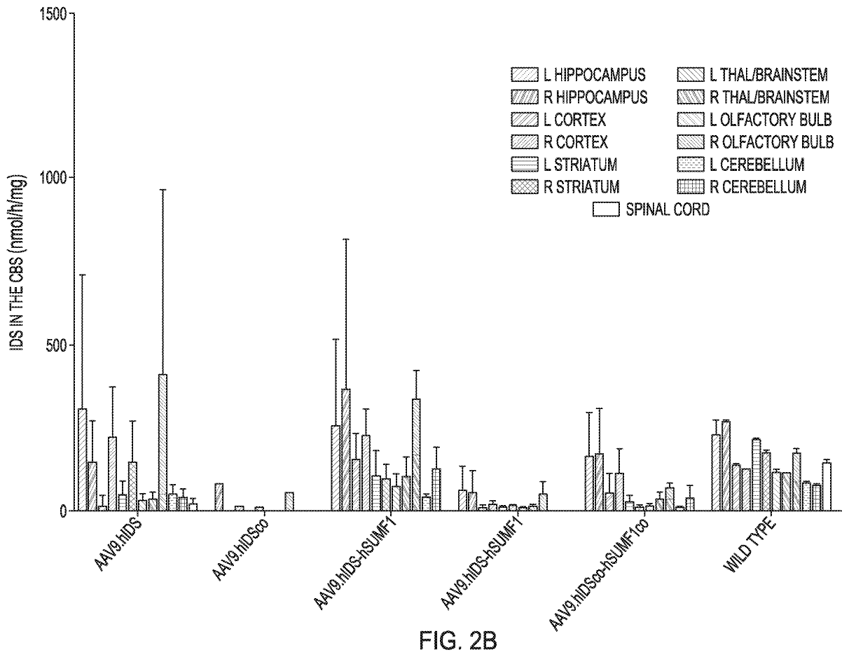

Another unexpected result from the initial pilot study described above is that while there was undetectable increase in IDS activity in the CNS, plasma IDS activity in both IV- and IT-treated groups was increased up to approximately 140-fold above the untreated wild-type level and persisted for at least 12 weeks post treatment (FIG. 2A). This result suggests that AAV vector was distributed to the peripheral circulation after IT injection into the cerebrospinal fluid (CSF). The presence of sustained enzyme activity for at least 12 weeks post injection (either IT or IV) also suggests that hIDS is presumably non-immunogenic for C57BL/6 mice.

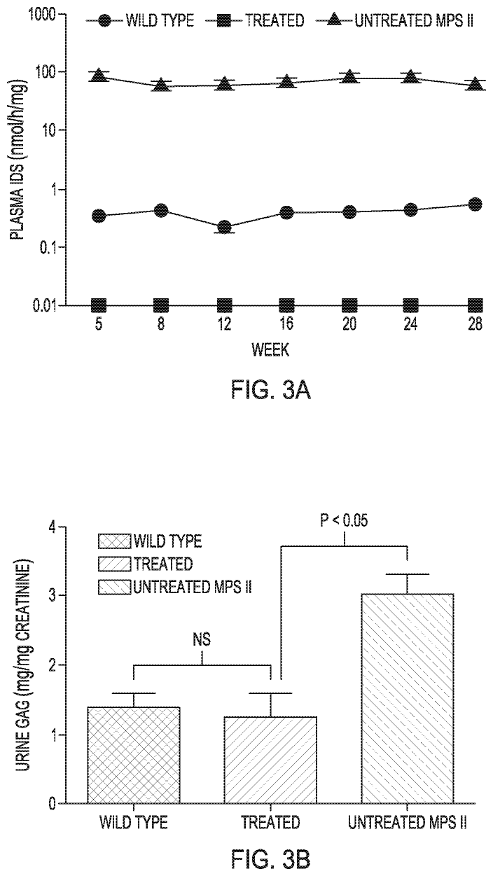

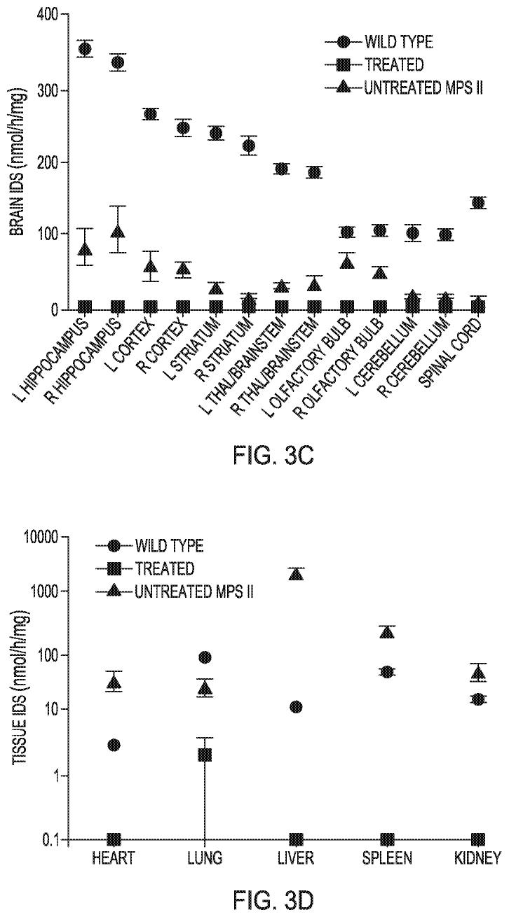

The same four vector constructs (AAV9.hIDS, AAV9.hIDSco, AAV9.hIDShSUMF1, and AAV9.hIDSco-hSUMF1co) were administered into immunocompetent MPS II mice by ICV injection-α procedure that supports a much higher level of transduction in the CNS than IT injection (Wolf et al., 2011). Immunosuppression of the MPS II test animal was not necessary, since we found that expression of human IDS does not elicit an immune response in C57BL/6 mice. An additional group of MPS II mice was injected ICV with a combination of two vectors-AAV9.hIDS (FIG. 1A) and AAV9.hSUMF1 (FIG. 1E)—at a 1:1 ratio (AAV9.hIDS+AAV9.hSUMF1; at a dose of 5×10¹⁰ vc total) to determine if there would be additional IDS activity when SUMF1 and IDS are both translated independently rather than relying on translation of SUMF1 from a downstream position by employing an IRES. Untreated wild-type littermates were used as controls. Six weeks post injection, the animals were euthanized, organs were harvested, and brains were microdissected to determine IDS activity. Animals injected with AAV9.hIDS, AAV9.hIDS-hSUMF1, or AAV9.hIDS+AAV9.h-SUMF1 showed levels of IDS activity approximately 10-40% of the wild-type level in most portions of the brain (FIG. 3). IDS activity was undetectable in all areas of the brain in MPS II mice (FIG. 3C). Animals injected with codon-optimized vector constructs showed mostly <10% of the wild-type level, so codon optimization of the IDS sequence did not result in a higher level of IDS activity in transduced tissues. There was no significant difference between AAV9.hIDS-injected animals and animals injected with AAV9.hIDS plus hSUMF1. Thus, co-delivery of hSUMF1 either on the same vector or on a separate vector did not enhance the level of IDS activity assessed. Vector AAV9.hIDS (FIG. 1A) was subsequently used for more extensive efficacy studies in ICV-administered MPS II mice, as described below, as neither the addition of SUMF1 nor the codon-optimization algorithm resulted in increased IDS activity compared to the native hIDS cDNA sequence.

Prevention of CNS and Peripheral Lysosomal Disease by ICV Administration of AAV9.hIDS Vector

A dose of 5.6×10¹⁰ AAV9. hIDS vc was infused into 8-week-old MPS II mice by ICV injection to achieve widespread CNS distribution of the vector through the CSF. As in the pilot study, plasma IDS activities up to 160-fold higher than wild type were observed in this larger cohort of ICV-treated MPS II animals, and this expression persisted throughout the experiment (28 weeks post injection; FIG. 3A).

Urine was collected at the end of the study (week 40 post injection) to evaluate the effect of long-term IDS expression on GAG excretion in the treated animals compared to wild-type and untreated MPS II mice. Urine GAG was significantly elevated in MPS II animals when compared to wild-type littermates (FIG. 3B; p<0.05). The treated animals demonstrated a significant reduction in urine GAG content (p<0.05) when compared to untreated littermates and were normalized when compared to the wild-type level (p>0.05).

At 10 months of age (40 weeks post injection), all mice were euthanized, and organs were harvested for analysis. IDS activity was undetectable in all areas of the brain and spinal cord of untreated MPS II mice (FIG. 3C). AAV9.hIDS-injected animals had IDS activity in all regions of the brain at approximately 9-28% of wild type, 53% in the olfactory bulb and 7% in the spinal cord (FIG. 3C). Although the vector was infused into the right ventricle of the brain, no significant difference in IDS activity was observed between the left and right hemispheres (p>0.05). Unlike the CNS, supraphysiological levels of enzyme activity were observed in all tested peripheral organs such as the heart, liver, spleen, and kidney (11-, 166-, 5—, and 3-fold, respectively; FIG. 3D), except in the lung where 34% of wild type was observed (FIG. 3D). This suggests that the vector was able to cross the BBB from the CNS into the circulation whereby it was taken up by and expressed in peripheral organs.

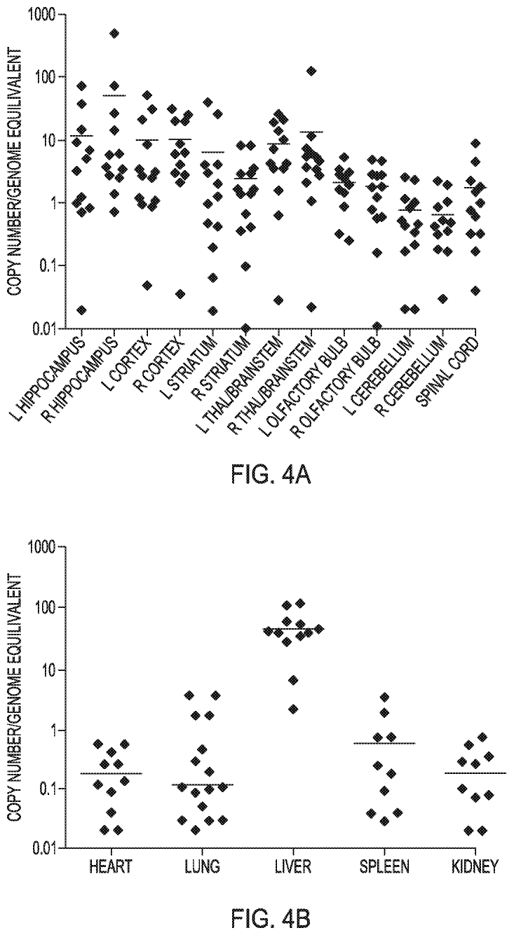

DNA was isolated from the same tissue homogenates and evaluated for vector distribution by qPCR. Consistent with Wolf et al. (2011), ICV infusion of AAV vector resulted in global distribution of vector in the CNS (FIG. 4A) and in all of the tested peripheral tissues (FIG. 4B). The highest vc number was observed in the right hippocampus (49 vc/ge), while similar vc were observed between the left and right hemispheres for all regions of the brain (FIG. 4A). Unlike the CNS, relatively low copy numbers were detected in most tested peripheral tissues, including the heart, lung, spleen, and kidney of the treated animals (<0.6 vc/ge; FIG. 4B), while high vc numbers were observed in the liver (44 vc/ge; FIG. 4B). This suggests that enzyme produced by the liver was released into the circulation where the tested peripheral organs took up the circulating enzyme (i.e., metabolic crosscorrection).

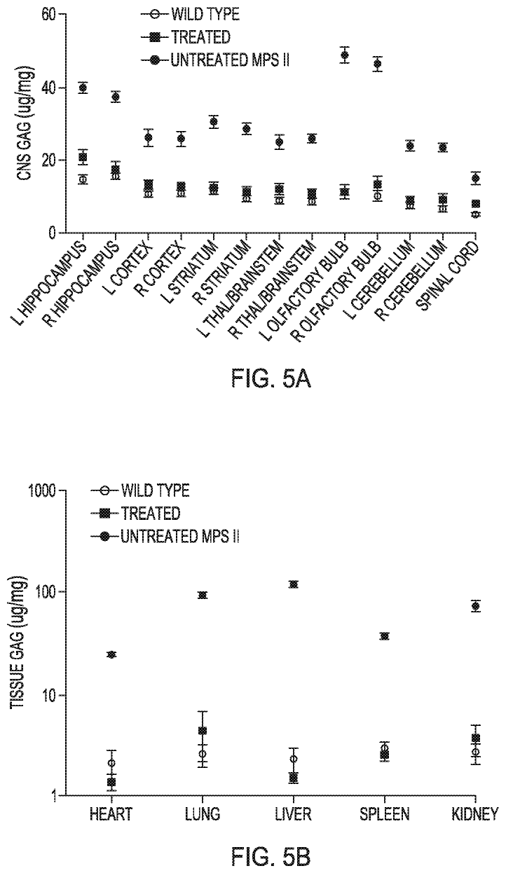

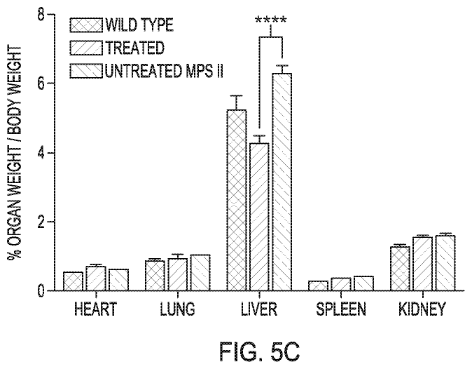

The global distribution and expression of IDS had a significant effect on accumulation of lysosomal storage materials. Elevation of lysosomal GAG content was observed in the CNS of untreated MPS II mice when compared to wild-type littermates (FIG. 5A; p<0.0001). Even though there was only 10-40% of wild-type IDS level in the CNS, GAG content in the CNS was normalized when compared to wild type (p<0.01; FIG. 5A). Similar to the CNS, significant elevation of lysosomal GAG content was observed in all tested peripheral organs of the untreated MPS II animals when compared to wild type (p<0.01; FIG. 5B). In contrast, significantly decreased GAG levels were observed in all tested peripheral tissues (FIG. 5B) of the treated mice when compared to the untreated group (p<0.01). Statistical analysis showed no significant difference in GAG content between wild-type animals and the treated group (p>0.05), which indicates that GAG content was normalized in the treated group.

The body weights of all mice were measured before sacrifice, and organs were weighed after the animals were perfused with 1× PBS, calculating the percentage of organ weight to body weight immediately post sacrifice. No significant difference was observed in the size of the heart, lung, spleen, or kidney among all groups. However, the liver of untreated MPS II mice was 20% larger than that of wild-type animals (6.2% and 5.2% of total body weight, respectively; p<0.001; FIG. 5C). In contrast, the liver of the treated MPS II mice was 68% smaller than the untreated group (4.2% and 6.2% of total body weight, respectively; p<0.0001; FIG. 5C). This result shows that normalization of GAG content in the liver in turn prevented hepatomegaly in the treated mice.

### Sustained Expression of IDS in the CNS Leads to Prevention of Neurocognitive Deficit in MPS II Mice

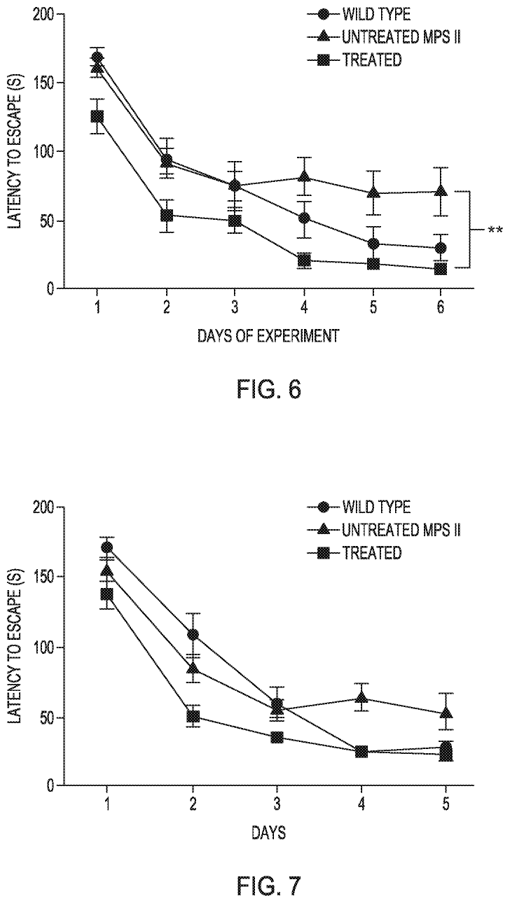

At 6 months of age (4 months post treatment), untreated MPS II mice, AAV9.hIDS-treated MPS II mice, and control normal littermates were evaluated for neurocognitive function in the Barnes maze, a test for spatial navigation and memory. The animals were subjected to a series of 3-minute trials, four trials per day for a course of 6 days. Wild-type littermates showed reduced latency to escape (30 s) on day 6, while untreated MPS II mice exhibited a significant deficit in learning this task (latency to escape reduced only to 71 s; p≤0.05 vs. normal littermates; FIG. 6). In contrast, the AAV9.hIDS-treated mice showed a marked reduction in latency to escape (25 s), significantly outperforming the untreated MPS II mice (p≤0.01) on days 5 and 6. In addition, there was no significant difference between the treated animals and wildtype littermates (p>0.05; FIG. 6). We conclude that sustained expression of IDS in the CNS prevented the emergence of neurocognitive deficits in MPS II mice when treated at a young age.

### DISCUSSION

In this study, AAV9.hIDS using a strong promoter was administered to the CNS of MPS II mice. Levels of IDS activity in the CNS were only 7-28% of wild type. In contrast, levels of IDS activity in the circulation and in tested peripheral organs were at least 2— and up to 170-fold higher than wild-type levels. Sustained IDS expression leads to global normalization of GAG content. Finally, sustained levels of IDS activity had a profound effect on preventing neurologic deterioration.

No significant increase in IDS activity in the CNS above the endogenous level was observed in IV- or IT-treated IDS+ mice, regardless of vector construct, route of administration, or mouse strain, even though IDS was expressed under regulation of the strong CB7 promoter (data not shown). However, the level of plasma IDS activity in IT-treated IDS+ mice was an average of 100-fold higher than the wild-type level, indicating successful IT administration of vector. Similar high levels of plasma IDS were observed when AAV9. hIDS was infused into MPS II mice via ICV injection (FIG. 2B and FIG. 3C), although in this case IDS activity in the CNS above baseline was observed in all 12 assayed portions of the brain (FIG. 3C). Similar IDS activities were reported from Motas et al. (in coronal sections of the brain (Motas et al., 2016)) and Hinderer et al. (2016) (whole brain), in which they observed approximately 20-40% of wild-type levels in the CNS. Here, a more extensive analysis of vector distribution and expression is reported in all different micro-dissected areas of the brain after ICV administration of AAV9.hIDS in MPS II mice.

Unlike Motas et al. (2016) and Hinderer et al. (2016), there was limited level of IDS activity achieved in the CNS after ICV administration of AAV9. hIDS. These limited levels of IDS activity are in stark contrast with the levels of IDUA activity observed in the CNS of MPS I mice after ICV injection of AAV9-IDUA vector, in which 100- to 1,000-fold higher than wild type levels of IDUA activity are observed (Belur et al., 2014). Supraphysiological levels of IDS (>1,000 nmol/h/mg) were also observed in the liver of ICV-administered animals in our study at a similar vc number that yielded 10- to 100-fold less IDS expression (10-100 nmol/h/mg) in the CNS. Highly relevant to the goal of CNS-directed gene therapy for MPS II, therefore, is this question: what is it that limits expression of IDS in the brain after highly efficient AAV-mediated IDS gene delivery?

One possibility is that SUMF1 activity might be rate limiting for the generation of active IDS in the brain. SUMF1 is required for the post-translational activation of lysosomal sulfatases, including IDS (Sabourdy et al., 2015). hIDS enzyme clearly was activated in tissues of the MPS II mouse, presumably by mouse SUMF1, but this process could be rate limiting in the brain if AAV-encoded hIDS protein is expressed in a large quantity but only a limited amount of hIDS becomes activated. For example, Fraldi et al. (2007) demonstrated that N-sulfoglucosamine sulfohydrolase activities were increased when the enzyme was co-expressed with SUMF1 in their MPS IIIA studies. Anticipating a potential limitation of SUMF1, hIDS and hSUMF1 were co-transduced either on the same construct (FIG. 1C) or on two separate vectors (FIGS. 1A and E), but no significant increase in hIDS activity was found compared to delivery of hIDS alone (FIG. 2B). hIDS activity was not enhanced by co-delivery with hSUMF1 in these experiments.

Another possibility is that the CB7 promoter might be limiting when compared to the endogenous IDS promoter. To further investigate this possibility, IDS activity (nmol/h/mg protein=unit) per vc was calculated for each tissue and compared to IDS activity per endogenous copy in male mice. IDS activities in the CNS of the treated mice were observed between 2 and 32 (mean of 8) units per vc compared to wild-type male mice, which express an average of 200 units per genome equivalent (approximately only 1-31% of wild-type level). By comparison, in the liver there was an average of 55 units of IDS activity per vc in the ICV-treated mice. From this, one might conclude that the CB7 promoter is not as robust as the endogenous IDS promoter in the brain. However, the lower level of vector-mediated IDS expression per vc versus endogenous expression most likely results from the presence of excess transcriptionally inactive AAV genomes remaining in the intracranial space post injection, possibly due to inefficient intracellularization. Nonetheless, the MPS II results contrast greatly with the results from our MPS I studies in which levels of IDUA activity at approximately 10- to 100-fold higher than wild type were observed in the CNS of mice administered AAV-hIDUA intrathecally (Belur et al., 2014), while heterozygous MPS I animals express approximately 6 units per genome equivalent in the brain (Ou et al., 2014). Therefore, it is feasible to achieve or exceed wild-type IDUA levels in the CNS of MPS I mice. The promoter that we used in the MPS II study was similar but not identical to the promoter used in the MPS I studies (Wolf et al., 2011; Belur et al., 2014), and the possibility that relative promoter strength may have contributed to the observed differences in outcome cannot be excluded.

Surprisingly, supranormal levels of enzyme activity were observed in all tested peripheral organs of the treated animals. The highest levels of IDS activity (164-fold wild type) were observed in the liver, which correlated with the highest number of vc found (48 vc/ge). In contrast, the tested peripheral organs (other than the liver) contained low levels of vector, but levels were higher than wild-type levels of IDS activity. The exceptionally high levels of IDS in the liver (FIG. 3D) lead to sustained high levels of IDS activity (up to 172-fold higher than wild type) in the circulation (FIG. 2A and FIG. 3A). Similar results have been reported in a MPS IIIA study and several MPS I studies using different types of vectors (Aronovich et al., 2009; Osborn et al., 2011; Haurigot 2013), along with two studies in MPS II mice (Motas et al., 2016; Hinderer et al., 2016). These results suggest that in the present study, the liver acts as an enzyme factory that produces an enormous amount of IDS and releases it into the circulatory system where it is subsequently taken up by the other organs in the periphery. Even though higher than wild-type levels of IDS enzyme activity were observed in the heart, spleen, and kidney in the treated mice, the vector levels in these organs were unexpectedly low (<0.6 vc/ge). This low transduction rate suggests that these organs took up circulating IDS, leading to metabolic cross-correction with higher levels of IDS than wild type.

Polito et al. observed no detectable IDS activity in the CNS after a single IV injection of AAV2/5 CMV-hIDS. However, they did observe partial correction of GAG content in the CNS (Polito et al., 2009). They speculated that only a minute fraction of high-level circulating hIDS produced from the liver penetrated the BBB into the CNS, with subsequent partial correction of GAGs. Similarly, in the current experiments exceptionally high levels of IDS were found in the circulation of AAV9.hIDS-treated mice, and yet only subphysiological activities of enzyme were observed in the CNS. This finding indicates that an insignificant amount of enzyme produced from the liver after ICV injection of vector was able to penetrate the BBB from the circulation into the CNS. This is consistent with the lack of increased IDS over wild-type levels in the CNS after IV administration of AAV9.hIDS in IDS+C57BL/6 mice (FIG. 2A). Therefore, the level of IDS observed in the brain most likely relied on transduction of the vector construct inside the CNS after ICV injection rather than from circulating enzyme. Further investigation is needed to achieve IDS levels comparable to or higher than the wild type in the CNS.

Sustained levels of enzyme expression after direct vector injection into the CNS have been shown to have profound effects on GAG reduction in several MPS studies irrespective of whether wild-type levels of those enzymes were achieved (Janson et al., 2014; Barnes, 1979; Belur et al., 2014; Motas et al., 2016; Ou et al., 2014). Similarly, although only subphysiological levels of IDS were observed in the CNS after ICV injection of AAV9.hIDS, there was a significant effect of the enzyme on GAG reduction that was greater than expected. Desnick et al. and Polito et al. speculated that only <5% of the wild-type level of lysosomal enzyme is needed to correct a GAG storage defect (Polito et al., 2009; Desnick et al., 2012). The present results are consistent with this speculation. After AAV9.hIDS treatment, all areas of the brain (FIG. 5A) and even the spinal cord (the organ with the lowest enzyme measured; 10.7 nmol/h/mg protein; 7.4% of wild type; FIG. 3C) showed significantly lower GAG content than in untreated MPS II mice (FIG. 5A). Statistical analysis revealed striking results, as GAG content in the CNS of the treated mice was normalized when compared to wild type. We also observed normalization of GAG content in the urine and in the heart, lung, liver, spleen, and kidney of the treated animals. These results indicate a sustained effect of IDS expression on correction of GAG content.

Roberts et al. demonstrated a direct relationship between GAG accumulation and liver size when injecting rodamine B, a GAG synthesis inhibitor, into MPS IIIA mice. They found that GAG content was decreased in the liver, leading to normalization of liver size (Roberts et al., 2006). Motas et al. observed a preventive effect on hepatomegaly after ICV administration of AAV9.hIDS into MPS II mice (Motas et al., 2016). Similarly, we also observed that the weight of the liver in treated mice was normalized after ICV injection of AAV9.hIDS (FIG. 5C). This result indicates a profound effect of sustained IDS expression leading to normalization of GAG content in the liver, which in turn prevents hepatomegaly in the treated mice.

Several MPS studies have demonstrated prevention of neurologic deficits after AAV-mediated gene transfer in mice (Wolf et al., 2011; Motas et al., 2016; Fu et al., 2011). We observed neurocognitive deficiency in untreated MPS II mice at 6 months of age. We also observed that sustained IDS expression in the CNS prevented the emergence of neurocognitive dysfunction in MPS II mice after ICV infusion of AAV9.hIDS (FIG. 6). Several studies have shown that the hippocampus is associated with neurocognition in rodents (Seeger et al., 2004; Paylor et al., 2001; Miyakawa et al., 2001). However, the possibility that physical impairment such as vision, olfactory sense, or motor neuron defects could also affect the results in the Barnes maze cannot be excluded, as rodents require all of the aforementioned physical capabilities in order to perform the required task in this test (Harrison et al., 2006). Additional behavioral analyses would support the observation that neurocognitive deficit plays a pivotal role in learning impairment of untreated MPS II mice and its prevention by AAV-mediated IDS gene transfer.

In conclusion, the present application characterizes the benefit of direct AAV9-mediated hIDS gene transfer to the CNS. The most important challenge emerging from this study is the limited level of AAV-mediated IDS expression achieved in the CNS in comparison with other tissues such as the liver, and in comparison with the expression of other therapeutic genes introduced into the CNS by AAV-mediated transduction such as IDUA (Wolf et al., 2011; Belur et al., 2014). Nonetheless, the MPS II data indicate that direct injection of AAV9.hIDS vector into the CNS resulted in efficient gene transfer that is key to treatment of MPS II and prevention of neurocognitive deficits. We also found that AAV9.hIDS vector was capable of crossing the BBB from the CNS into the circulation, resulting in global transduction of the vector outside of the CNS, providing long-term expression of IDS enzyme systemically. Even though the IDS activities in the brain were lower than expected, this study nonetheless supports the notion from previous studies (Polito et al., 2009; Desnick et al., 2012) that <10% of the wild-type level of IDS is needed to prevent GAG storage accumulation. In addition, sustained IDS expression corrected the accumulation of GAG in liver and subsequently prevented the emergence of hepatomegaly. Finally, our results reinforce the importance of sustained IDS expression in the CNS in preventing the emergence of neurologic deficits when animals are treated at a young age. We anticipate that this study that this study will contribute to the field in developing a long-term effective treatment with neurological benefits for MPS II patients.

### Example IV

Mucopolysaccharidosis type I (MPS I) is an inherited autosomal recessive metabolic disease caused by deficiency of α-L-iduronidase (IDUA), resulting in accumulation of heparin and dermatan sulfate glycosaminoglycans (GAGs). Individuals with the most severe form of the disease (Hurler syndrome) suffer from neurodegeneration, mental retardation, and death by age 10. Current treatments for this disease include allogeneic hematopoietic stem cell transplantation (HSCT) and enzyme replacement therapy (ERT). However, these treatments are insufficiently effective in addressing CNS manifestations of the disease.

The goal is to improve therapy for severe MPS I by supplementing current ERT and HSCT with IDUA gene transfer to the CNS, thereby preventing neurological manifestations of the disease. In this study, the ability of intravenously administered AAV serotypes 9 and rh10 (AAV9 and AAVrh10) to cross the blood brain barrier for delivery and expression of the IDUA gene in the CNS was tested. 4-5 month old adult MPS I animals were infused intravenously via the tail vein with either an AAV9 or AAVrh10 vector encoding the human IDUA gene. Blood and urine samples were collected on a weekly basis until the animals were sacrificed at 10 weeks post-injection. Plasma IDUA activities in treated animals were close to 1000-fold higher than that of heterozygote controls at 3 weeks post-injection. Brains, spinal cords, and peripheral organs were analyzed for IDUA activity, clearance of GAG accumulation, and IDUA immunofluorescence of tissue sections. Treated animals demonstrated widespread restoration of IDUA enzyme activity in all organs including the CNS. These data demonstrate the effectiveness of systemic AAV9 and AAVrh 10 vector infusion in counteracting CNS manifestations of MPS I.

### Example V

Gene transfer offers enormous potential for therapy of the mucopolysaccharidoses. Studies have focused on achieving high-level expression of alpha-L-iduronidase (IDUA) in the CNS of MPS I mice, where it was observed enzyme up to 1000-fold greater than wild-type (WT) in the brain after intracerebroventricular (ICV) infusion of AAV9 transducing the human IDUA gene. Intrathecal (IT) infusion of AAV9 vector also resulted in high-level IDUA expression (10- to 100-times that of wild-type) throughout the brain. All routes of administration normalized glycosaminoglycan levels in all areas of the brain and prevented the emergence of neurocognitive deficiency at 4-5 months of age as assessed in the Barnes maze. WT mice expressed much higher levels of endogenous iduronate sulfatase (IDS) than IDUA in the brain, and in animals infused IT with AAV9 transducing the human IDS gene, the level of IDS in the brain was indistinguishable from WT. After ICV infusion of AAV9-IDS vector in MPS II mice there was sufficient expression of IDS to reduce GAG accumulation to near wild-type levels and prevent the emergence of neurocognitive dysfunction, but the level of IDS never achieved that of WT in the brain . . .

### Example VI

Hunter Syndrome (Mucopolysaccharidosis type II; MPS II) is an X-linked recessive inherited lysosomal disease caused by deficiency of iduronate-2-sulfatase (IDS) and accumulation of glycosaminoglycans (GAGs) in tissues, resulting in skeletal dysplasias, hepatosplenomegaly, cardiopulmonary obstruction, and neurologic deterioration. Patient standard of care is enzyme replacement therapy (ERT) although ERT is not associated with neurologic improvement. In a mouse model of IDS deficiency, intracerebroventricular (ICV) administration of AAV9.hIDS into young 8-week old mice resulted in corrective levels of hIDS enzyme activity, reduction of GAG storage to near WT-levels and prevention of neurocognitive dysfunction, compared to IDS deficient control littermates. Since the emergence of neurologic manifestations could be prevented in young adults, it was hypothesized that older adult MPS II animals treated at 4 months of age by ICV administration of AAV9.hIDS would recover neurobehavioral function and show corrected levels of IDS enzyme activity and GAG storage. By 4 weeks post-ICV injection, IDS enzyme activity in the circulation was 1000-times that of WT-levels (305+/−85nmol/hr/ml compared to 0.39+/−0.04nmol/hr/ml). At 36 weeks of age, the treated animals were tested for neurocognitive function in the Barnes maze. Performance of the treated animals was indistinguishable from that of unaffected littermates and significantly improved compared to untreated MPS II mice. Cognitive function that is lost by 4 months of age can thus be restored in MPS II mice by delivery of AAV9 encoding IDS to the cerebrospinal fluid. The implication of these results is the prospect that human MPS II may be treatable after the development of neurologic manifestations by AAV mediated IDS gene transfer to the CNS.

### Example VII

MPSII is a rare X-linked lysosomal storage disease. MPSII patients have a deficiency in IDS and accumulate GAGs Clinical manifestations include coarse facial features, short stature, dysostosis multiplex, joint stiffness, skeletal dysplasias and neuroncompression, organomegaly, retinal degeneration, cardiac/respiratory obstruction, pebbled skin and intellectual disability (severe form). Current treatments (HSCT and ERT) are lacking in that in HSCT there is a very low level of enzyme expression in HSCT (MPSI) and so it is less likely to provide a benefit in reversing neurological deficit, and in ERT enzymes are rapidly depleted and do not cross blood brain barrier, and no neurological improvement.

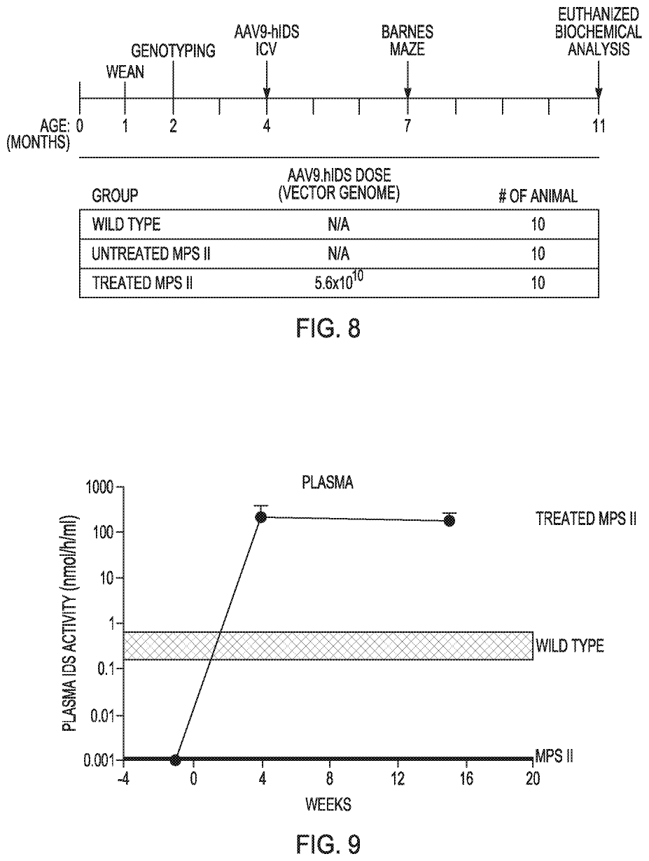

In order to investigate whether treatment with AAV-IDS of older MPSII animals that already manifested neurological deficit has a beneficial effect, 4 month old MPSII mice were ICV administered AAV-hIDS (FIG. 8).

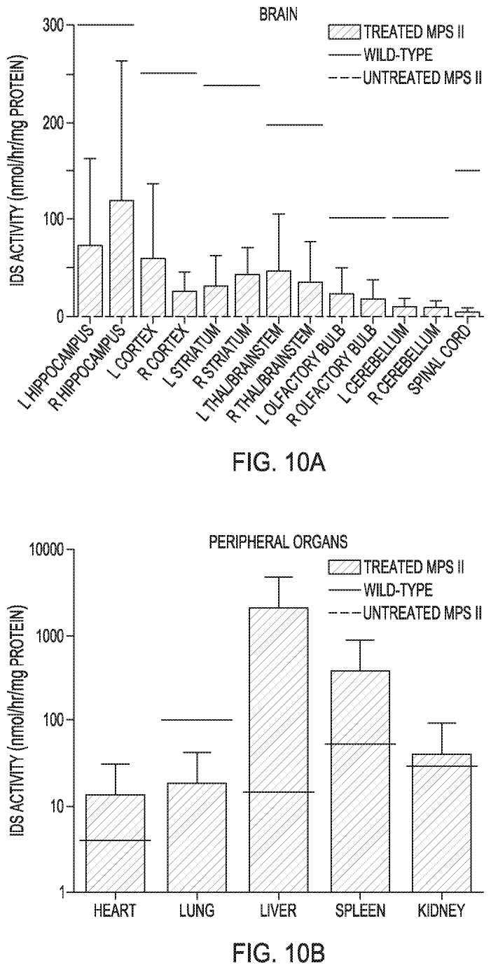

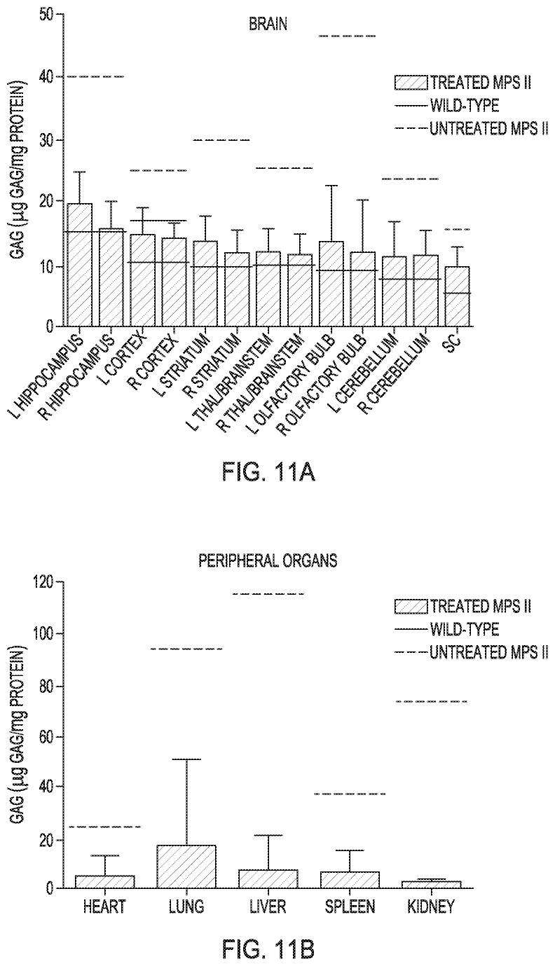

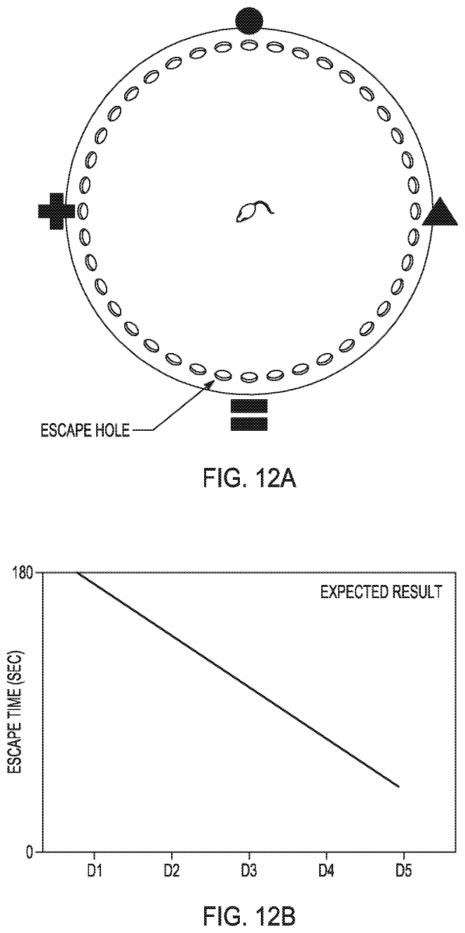

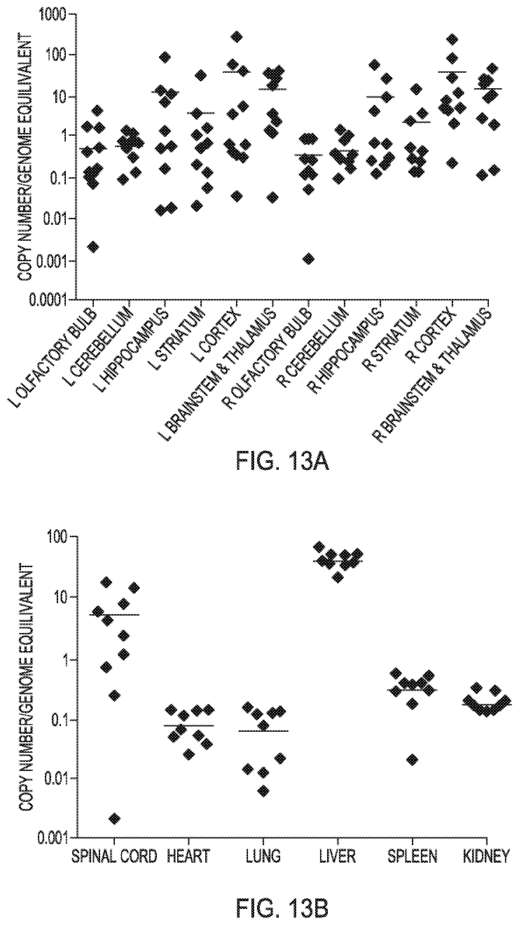

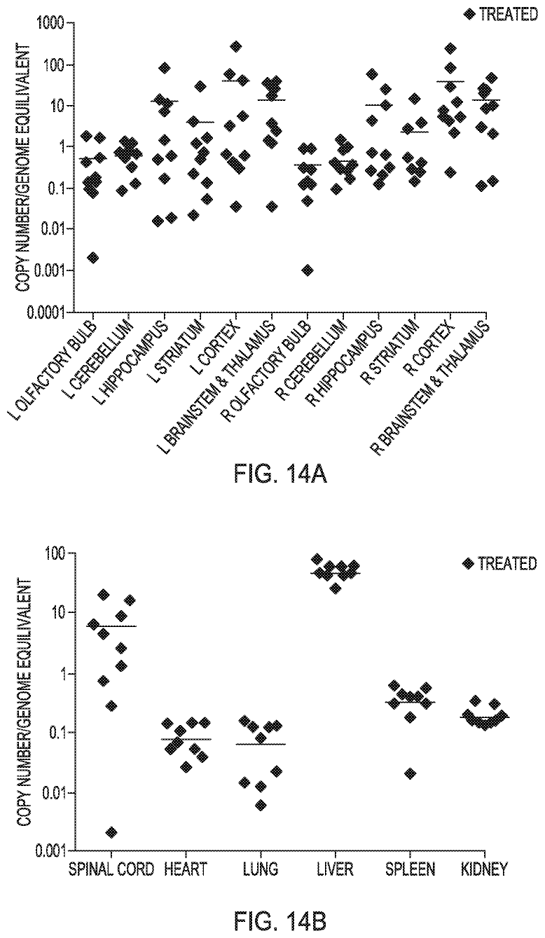

MPSII animals treated at 4 months by AAV9.hIDS ICV injection exhibited 500× WT IDS enzyme activity in plasma (FIG. 9), about 100× WT IDS enzyme activity in liver and elevated enzyme activity in the brain, e.g., hippocampus about 1/3 WT levels (FIG. 10), and GAG levels restored to WT levels in all tissues (FIG. 11), and treatment restored neurocognitive function (FIG. 7).

### Example VIII

### AAV Vector-Mediated Iduronidase Gene Delivery in a Murine Model of Mucopolysaccharidosis Type: Comparing Different Routes of Delivery to the CNS

Mucopolysaccharidosis type I (MPS I) is an inherited metabolic disorder caused by deficiency of the lysosomal enzyme alpha-L-iduronidase (IDUA). Systemic and abnormal accumulation of glycosaminoglycans is associated with growth delay, organomegaly, skeletal dysplasia, and cardiopulmonary disease. Individuals with the most severe form of the disease (Hurler syndrome) suffer from neurodegeneration, mental retardation, and early death. The two current treatments for MPS I (hematopoietic stem cell transplantation and enzyme replacement therapy) cannot effectively treat all central nervous system (CNS) manifestations of the disease.

With respect to gene therapy, it was previously demonstrated that intravascular delivery of AAV9 in adult mice does not achieve widespread direct neuronal targeting (see Foust et al, 2009). Previous work also showed that direct injection of AAV8-IDUA into the CNS of adult IDUA-deficient mice resulted in a low frequency or a poor level of transgene expression. The following examples, which use a pre-clinical model for the treatment of MPS1, surprisingly demonstrate that direct injection of AAV9-IDUA into the CNS of immunocompetent adult IDUA-deficient mice resulted in IDUA enzyme expression and activity that is the same or higher than IDUA enzyme expression and activity in wild-type adult mice.

### Methods

AAV9-IDUA preparation. The AAV-IDUA vector construct (MCI) has been previously described (Wolf et al., 2011) (mCags promoter). AAV-IDUA plasmid DNA was packaged into AAV9 virions at the University of Florida Vector Core, yielding a titer of 3×10¹³ vector genomes per milliliter.

ICV infusions. Adult Idua-/-mice were anesthetized using a cocktail of ketamine and xylazine (100 mg ketamine+10 mg xylazine per kg) and placed on a stereotactic frame. Ten microliters of AAV9-IDUA were infused into the right-side lateral ventricle (stereotactic coordinates AP 0.4, ML 0.8, DV 2.4 mm from bregma) using a Hamilton syringe. The animals were returned to their cages on heating pads for recovery.

Intrathecal infusions. Infusions into young adult mice were carried out by injection of 10 μL AAV vector containing solution between the L5 and L6 vertebrae 20 minutes after intravenous injection of 0.2 mL 25% mannitol.

Immunotolerization. Newborn IDUA deficient mice were injected through the facial temporal vein with 5 μL containing 5.8 μg of recombinant iduronidase protein (Aldurazyme), and then the animals were returned to their cage.

Cyclophosphamide immunosuppression. For immunosuppression, animals were administered cyclophosphamide once per week at a dose of 120 mg/kg starting one day after infusion with AAV9-IDUA vector.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C. Samples were thawed and homogenized in 1 mL of PBS using a motorized pestle and permeabilized with 0.1% Triton X-100. IDUA activity was determined by fluorometric assay using 4MU-iduronide as the substrate. Activity is expressed in units (percent substrate converted to product per minute) per mg protein as determined by Bradford assay (BioRad).

Tissues. Tissue homogenates were clarified by centrifugation for 3 minutes at 13,000 rpm using an Eppendorf tabletop centrifuge model 5415D (Eppendorf) and incubated overnight with proteinase K, DNase1, and Rnase. GAG concentration was determined using the Blyscan Sulfated Glycosaminoglycan Assay (Accurate Chemical) according to the manufacturer's instructions.

### Results

Iduronidase-deficient mice were administered AAV either intracerebroventricularly (ICV) or intrathecally (IT). To prevent immune response, animals were either immunosuppressed with cyclophosphamide (CP), immunotolerized at birth by intravenous administration of human iduonidase protein (aldurazyme), or the injections were carried out in NOD-SCID immunodeficient mice that were also iduronidase deficient. Animals were sacrificed at the indicated time post-treatment, the brains were microdissected and extracts assayed for iduronidase activity.

Immunodeficient, IDUA deficient animals that were injected ICV with AAV-IDUA vector exhibited high levels of IDUA expression (10 to 100 times wild type) in all areas of the brain, with the highest level observed in the brain stem and thalamus (“rest”).

Immunosuppressed animals administered AAV vector by ICV route had a relatively lower level of enzyme in the brain compared to the immunodeficent animals. Note that immunosuppression may have been compromised in these animals because CP was withdrawn 2 weeks before sacrifice due to poor health.

Immunosuppressed animals were administered AAV vector by the IT route. Immunotolerized animals administered AAV vector ICV exhibited widespread IDUA activity in all parts of the brain, similar to that observed in the immunodeficient animals, indicating the effectiveness of the immunotolerization procedure.

GAG storage material was assayed in the different sections of the brain for all four of the test groups. For each group, the mean of each portion of the brain is shown on the left, the values for each of the individual animals is shown on the right. IDUA deficient animals (far left) contained high levels of GAG compared to wild type animals (magenta bar). GAG levels were at wild-type or lower than wild type for all portions of the brain in all groups of AAV-treated animals. GAG levels were slightly although not significantly higher than wild-type in cortex and brainstem of animals administered AAV9-IDUA intrathecally.

### Conclusions

The results show high and widespread distribution of IDUA in the brain regardless of the route of delivery (ICV or IT) although IDUA expression in striatum and hippocampus was lower in animals injected IT versus ICV. There appears to be an immune response since immune deficient mice have higher levels of expression than immunocompetent mice. With regard to ICV injection, when CP was withdrawn early, IDUA expression is lower. In addition, immunotolerization was effective in restoring high levels of enzyme activity. Further, GAG levels were restored to normal in all treated experimental groups of mice.

### Example IX

### Methods

AAV9-IDUA Preparation. AAV-IDUA plasmid was packaged into AAV9 virions at either the University of Florida vector core, or the University of Pennsylvania vector core, yielding a titer of 1-3×10¹³ vector genomes per milliliter.

ICV infusions. See Example VIII.

Intrathecal infusions. See Example VIII.

Immunotolerization. As in Example VIII except: for multiple tolerizations, newborn IDUA deficient mice were injected with the first dose of Aldurazyme in the facial temporal vein, followed by 6 weekly injections administered intraperitoneally.

Cyclophosphamide immunosuppression. See Example VIII.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C.

Tissue IDUA activity. Tissue samples were thawed and homogenized in saline in a tissue homogenizer. Tissue homogenates were clarified by centrifugation at 15,000 rpm in a benchtop

Eppendorf centrifuge at 4° C. for 15 minutes. Tissue lysates (supernatant) were collected and analyzed for IDUA activity and GAG storage levels.

Tissue GAG levels. Tissue lysates were incubated overnight with Proteinase K, RNase and DNase. GAG levels were analyzed using the Blyscan Sulfated Glycosaminoglycan Assay according to the manufacturer's instructions.

IDUA Vector copies. Tissue homogenates were used for DNA isolation and subsequent QPCR, as described in Wolf et al. (2011).

### Results

Animals were administered AAV9-IDUA vector either by intracerebroventricular (ICV) or intrathecal (IT) infusion. Vector administration was carried out in NOD-SCID immunodeficient (ID) mice that were also IDUA deficient, or in IDUA deficient mice that were either immunosuppressed with cyclophosphamide (CP), or immunotolerized at birth by a single or multiple injections of human iduronidase protein (Aldurazyme). All vector administrations were carried out in adult animals ranging in age from 3-4.5 months. Animals were injected with 10 μL of vector at a dose of 3×10¹¹ vector genomes per 10 microliters.

IDUA enzyme activities in intracranially infused, immunodeficient, IDUA deficient mice were high in all areas of the brain, ranging from 30- to 300-fold higher than wild type levels. Highest enzyme expressions were seen in thalamus and brain stem, and in the hippocampus.

Animals that were injected intracranially and immunosuppressed with cyclophosphamide (CP) demonstrated significantly lower levels of enzyme activity than other groups. However, CP administration in this case had to be withdrawn 2 weeks prior to sacrifice due to poor health of the animals.

IDUA enzyme levels in animals tolerized at birth with IDUA protein (Aldurazyme) and administered vector intracranially were high in all parts of the brain that ranged from 10- to 1000-fold higher than wild type levels, similar to levels achieved in immunodeficient animals, indicating the effectiveness of the immunotolerization procedure.

IDUA enzyme levels in mice that were injected intrathecally and administered CP on a weekly basis were elevated and were observed in all parts of the brain, especially in the cerebellum and the spinal cord. Levels of enzyme were the lowest in the striatum and hippocampus with activities at wild type levels.

IDUA deficient mice were tolerized with Aldurazyme as described, and injected with vector intrathecally. There was widespread IDUA enzyme activity in all parts of the brain, with highest levels of activity in the brain stem and thalamus, olfactory bulb, spinal cord and the cerebellum. Similarly, the lowest levels of enzyme activity were seen in the striatum, cortex and hippocampus.

Control immunocompetent IDUA deficient animals were infused with vector intrathecally, without immunosuppression or immunotolerization. The results indicate that although enzyme activities were at wild type levels or slightly higher, they are significantly lower than what was observed in animals that underwent immunomodulation. The decreases in enzyme levels were especially significant in the cerebellum, olfactory bulb and thalamus and brain stem, areas that expressed the highest levels of enzyme in immunomodulated animals.

Animals were assayed for GAG storage material. All groups demonstrated clearance of GAG storage, with GAG levels similar to that observed in wild type animals. Animals that were immunosuppressed and injected with AAV9-IDUA vector intrathecally had GAG levels in the cortex that were slightly higher than wild type, but still much lower than untreated IDUA deficient mice.

The presence of AAV9-IDUA vector in animals that were immunotolerized and injected with vector either intracranially or intrathecally was evaluated by QPCR. IDUA copies per cell were higher in animals infused intracranially in comparison with animals infused intrathecally, which is consistent with the higher level of enzyme activity seen in animals injected intracranially.

### Conclusions

High, widespread, and therapeutic levels of IDUA were observed in all areas of the brain after intracerebroventricular and intrathecal routes of AAV9-IDUA administration in adult mice. Enzyme activities were restored to wild type levels or slightly higher in immunocompetent IDUA deficient animals infused with AAV-IDUA intrathecally. Significantly higher levels of IDUA enzyme were observed for both routes of vector injection in animals immunotolerized starting at birth by administration of IDUA protein.

### Example X

Mucopolysaccharidosis type II (MPS II; Hunter Syndrome) is an X-linked recessive inherited lysosomal storage disease caused by deficiency of iduronate-2-sulfatase (IDS) and subsequent accumulation of glycosaminoglycans (GAGs) dermatan and heparan sulphate. Affected individuals exhibit a range in severity of manifestations physically, neurologically, and shortened life expectancy. For example, affected individuals exhibit a range in severity of manifestations such as organomegaly, skeletal dysplasias, cardiopulmonary obstruction, neurocognitive deficit, and shortened life expectancy. There is no cure for MPS II at the moment. Current standard of care is enzyme replacement therapy (ELAPSRASE; idursulfase), which is used to manage disease progression. However, enzyme replacement therapy (ERT) does not result in neurologic improvement. As hematopoetic stem cell transplantation (HSCT) has not shown neurologic benefit for MPS II, there is currently no clinical recourse for patients exhibiting neurologic manifestations of this disease, and new therapies are desperately needed.

AAV9 vectors are developed for delivery of the human IDS coding sequence (AAV9-hIDS) into the central nervous system of MPS II mice to restore IDS levels in the brain and prevent the emergence of neurocognitive deficits in the treated animals. In particular, a series of vectors were generated that encode human IDS with or without the human sulfatase modifying factor-1 (SUMF-1), required for activation of the sulfatase active site. Three routes of administration were used in these experiments: Intrathecal (IT). Intracerebroventricular (ICV) and Intravenous (IV). No significant difference in the enzyme level was found between mice that were treated with AAV9 vector transducing hIDS alone and mice that were treated with AAV9 vector encoding human IDS and SUMF-1, regardless of the route of administration. IT-administrated NOD.SCID (IDS Y+) and C57BL/6 (IDS Y+) did not show elevated IDS activity in the brain and spinal cord when compared to untreated animals, while plasma showed ten-fold higher (NOD. SCID) and 150-fold higher (C57BL/6) levels than untreated animals. IDS-deficient mice intravenously administered AAV9-hIDS exhibited IDS activities in all organs that were comparable to wild type.

Moreover, the plasma of IV injected animals showed enzyme activity that was 100-fold higher than wild type. IDS-deficient mice administered AAV9-hIDUA ICV showed IDS activities comparable to wild type in most areas of the brain and peripheral tissues, while some portions of the brain showed two-to four-fold higher activity than wild type. Furthermore, IDS activity in plasma was 200-fold higher than wild type. Surprisingly, IDS enzyme activity in the plasma of all treated animals showed persistence for at least 12 weeks post injection; therefore, IDS enzyme was not immunogenic at least on the C57BL/6 murine background. Additional neurobehavioral testing was conducted using the Barnes maze to differentiate neurocognitive deficits of untreated MPS II animals from that of wild type littermates. It was found that the learning capability of affected animals is distinctively slower than that observed in littermates. Thus, Barnes maze is used to address the benefit of these therapies in the MPS II murine model. These results indicate potential of therapeutic benefit of AAV9 mediated human IDS gene transfer to the CNS to prevent neurologic deficiency in MPS II.

In summary, intracerebroventricular (ICV) injection of AAV9-hIDS resulted in systemic correction of IDS enzyme deficiency, including wild-type levels of IDS in the brain. Co-delivery of hIDS with hSUMF-1 did not increase IDS activity in tissues. hIDS expression was non-immunogenic in WT and MPS II C57BL/6 mice.

The following provides further details in this regard.

Mucopolysaccharidosis type II (MPS II, Hunter syndrome) is a rare x-linked recessive lysosomal disorder caused by defective Iduronate-2-sulfatase (IDS) resulting in accumulation of heparan sulfate and dermatan sulfate glycosaminoglycans (GAGs). Enzyme replacement is the only FDA-approved therapy available for MPS II, but it is expensive and does not improve neurologic outcomes in MPS II patients. As described below, this study evaluated the effectiveness of IDS-encoding adeno-associated virus (AAV) vector encoding human IDS delivered intracerebroventricularly in a murine model of MPS II. Supraphysiological levels of IDS were observed in the circulation (160-fold higher than wild type) for at least 28 weeks post-injection and in most tested peripheral organs (up to 270-fold) at 10 months postinjection. In contrast, only low levels of IDS were observed (7% to 40% of wild type) in all areas of the brain. Sustained IDS expression had a profound effect on normalization of GAG in all tested tissues and on prevention of hepatomegaly. Additionally, sustained IDS expression in the CNS had a prominent effect in preventing neurocognitive deficit in MPS II mice treated at two months of age. The present study demonstrates that CNS-directed, AAV9 mediated gene transfer is a potentially effective treatment for Hunter syndrome as well as other monogenic disorders with neurologic involvement.

### INTRODUCTION

The mucopolysaccharidoses (MPSs) are a group of lysosomal disorders caused by deficiency of any one of 11 lysosomal hydrolase that catalyze the breakdown of glycosaminoglycans (GAGs). MPS type II (MPS II; Hunter syndrome), is an X-linked recessive caused by deficiency of iduronate-2-sulfatase (IDS) with subsequent accumulation of substrate (GAGs) in tissues of affected individuals associated with hepatosplenomegaly, skeletal dysplasia, joint stiffness, and airway obstruction. In severe cases, affected individuals exhibit neurocognitive deficits and succumb to the illness in adolescence. The current and only treatment available for MPS II is enzyme replacement therapy (ERT), which is used to mitigate disease progression but without neurologic improvement. Hematopoietic stem cell transplantation, which has been shown to provide long-term benefits for MPS I (Whitley et al., 1993), has not been reported to ameliorate neurodegenerative disease in severe cases of MPS II (McKinnis et al. 1996; Vellodi et al. 2015;

### Hoogerbrugge et al. 1995).

The Sleeping Beauty (SB) transposon system and minicircles are two non-viral gene therapy platforms that have been successfully used in mice for systemic diseases such as MPS type I and type VII (Aronovich et al. 2009; Aronovich et al. 2007; Osborn et al. 2011). Despite being efficient and providing sustained expression in vivo (Aronovich et al. 2007; Chen 2003), the major drawback of these systems is the inability to penetrate the BBB (Aronovich and Hackett 2015) which has not yet been resolved. This limits the effectiveness of non-viral gene therapy systems for the CNS.

Various viral vectors have been extensively studied in gene therapy clinical trials for many diseases because their potency and sustained expression (Kaufmann et al. 2013). Amongst these vehicles, adeno-associated viral vectors (AAVs) have been shown to be promising candidates for clinical trials in mediating gene transfer for monogenic disorders (Tanaka et al. 2012; Bennett et al. 2012; Nathwani et al. 2014). Unlike other AAV serotypes, adeno-associated viral vector serotype 9 (AAV9) has been demonstrated in many animal models to not only efficiently transduce the CNS and peripheral nervous tissues (PNS), but also penetrate the BBB and transduce various cell types in peripheral tissues (Duque et al., 2009; Foust et al., 2009; Huda et al., 2014; Schuster et al., 2014). Thus AA9 outperforms other viral vectors as a candidate for systemic correction including CNS for monogenic disorders such as MPS II. Herein is reported the effectiveness of CNS-directed, AAV9 mediated human IDS gene transfer to correct IDS deficiency and prevent neurocognitive impairment in a murine model of MPS II.

### Materials and Methods

AAV vector assembly and packaging. All vectors were constructed, packaged, and purified at the Penn Vector Core (Philadelphia, PA) and provided by REGENXBIO Inc. (Rockville, MD). In brief, the expression cassettes contained a chicken beta-actin (CB7) promoter with cytomegalovirus (CMV) enhancer followed by hIDS or human sulfatase modifying factor 1 (hSUMF1), rabbit beta-actin polyadenylation signal on the backbone of AAV2 inverted terminal repeats (ITR) on both 3′- and 5′-ends. Co-expression constructs included an internal ribosome entry site (IRES) positioned between IDS and SUMF1 to initiate translation of SUMF1 downstream of the IRES. In this study, five different vector constructs were investigated: AAV9 expressing human IDS alone (AAV9.hIDS); AAV9 expressing codon-optimized human IDS (AAV9.hIDSco); AAV9 coexpressing human IDS and human SUMF1 (AAV9.hIDS-hSUMF1); AAV9 coexpressing codon-optimized human IDS and codon-optimized human SUMF1 (AAV9.hIDScohSUMF1co); AAV9 expressing human SUMF1 alone (AAV9.hSUMF1). AAV vectors were packaged by co-transfecting 3 plasmids: AAV cis, AAV trans (pAAV2/9 rep and cap), and adenovirus helper (pAdAF6), into HEK 293 cells (Lock et al. 2010). AAV vector was then purified from supernatants using a Profile II depth filter and concentrated by tangential flow filtration (TFF). The concentrated feed stock was reclarified by iodixanol gradient centrifugation, then reconcentrated using a TFF cassette with a 100-kDa MWCO HyStream screen channel membrane. The purified vector was then tested for purity by SDS-PAGE and for potency by qPCR (Lock et al. 2010).

Animal care and husbandry. All animal care and experimental procedures were conducted under approval of the Institutional Animal Care and use Committee (IACUC) of the University of Minnesota. NOD.SCID mice were purchased from The Jackson Laboratory and C57BL/6 wild-type mice were purchased from National Cancer Institute. C57BL6 iduronate-2-sulphatase knockout (IDS KO) mice were kindly provided by Dr. Joseph Muenzer (University of North Carolina, NC, USA) and maintained under specific pathogen-free conditions at the Research Animal Resources (RAR) facilities of the University of Minnesota. MPS II male pups (IDS−/0) were generated by breeding heterozygous (IDS^{+/-}) females to wild type (IDS+/0) C57BL/6 males. All pups were genotyped by PCR.

AAV vector administration. For intrathecal injections, eight-week old mice were injected with a dose of 5.6×10¹⁰ vector genomes (vg) of AAV9 vector between the L5 and L6 vertebrae. The injection was performed in conscious animals in a 10-15 second duration. For intravenous injections animals were briefly restrained and injected via tail-vain with a dose of 5.6×10¹⁰ vg. Intracerebroventricular injections were carried out in adult 8-week old mice.

Briefly, animals were injected intraperitoneally with 6 μl of ketamine/xylazine mixture (36 mg/ml ketamine, 5.5 mg/ml xylazine) to produce deep anesthesia and then mounted in a stereotactic frame (Kopf Model 900). An incision was made to expose the cranium, small hole was drilled as a site for the injection, and then a Hamilton syringe (Model 701) was used to carry out the infusion at a rate approximately 0.5 μL per minute by hand. The syringe was left in place for an additional 3 minutes, then slowly withdrawn over a period of at least 2 minutes. The scalp was sutured after completion of the injection, and after recovery from the anesthesia the mouse was then returned to standard housing. All of the mice received a 3-day course of Ketoprofen 2.5 mg/kg subcutaneously and Baytril 5 mg/kg intraperitoneally to prevent infection and inflammation post-surgery.

Sample collection and preparation. Blood was collected by submandibular puncture using sterile 5 mm lancets (Goldenrode™) into Microvette® heparinized coated tubes (SARSTEDT AG & Co.) and centrifuged in an Eppendorf centrifuge 5415D at 7000 rpm for 10 minutes. Plasma was collected and stored at −20° C. to −80° C. for IDS assay. Urine was collected and stored at −20° C. until used for creatinine and GAG assay. Organs were harvested by first determining animal weight using an OHAUS® CS 200 scale before they were euthanized using a CO₂ fume chamber at 2 liter/min for 3 minutes. The animals were perfused with 60 mL of 1×PBS in a 60 ml syringe (BD) with a SURFLO® winged infusion set (TERUMO®) size 23G x.” by hand-pressure. Heart, lung, liver, spleen, kidney, and spinal cord were harvested. The harvested peripheral organs were weighed using a Sartorius BP 61S scale. Brain was micro-dissected into left and right cerebellum, cortex, hippocampus, striatum, olfactory bulb, and thalamus/brainstem. The organs were immediately snap frozen and stored at −70° C. until further tissue processing.

For tissue processing, cerebellum, hippocampus, striatum, and olfactory bulb were added into pre-assigned 1.5 mL locked-cap microtubes (EPPENDORF) containing 1 scoop (0.2 g/scoop) of 0.5 mm glass beads (NEXT ADVANCE) in 250 μl sterile saline solution. Thalamus/brainstem, cortex and spinal cord were added into assigned locked-cap microtubes containing 2 scoops of 0.5 mm glass beads in 400 μl of sterile saline solution. Half of the lung and the whole spleen were added into the assigned tubes containing 2 scoops of 0.9-2.0 mm stainless steel blend (0.6 g/scoop) in 400 μl of saline solution. Heart, ~0.3 g liver, and one kidney were added into assigned tubes containing 3 scoops (a mixture of 2 scoops of 0.9-2.0 mm stainless steel blend (0.6 g/scoop) and 1 scoop of 3.2 mm stainless steel beads (0.7 g/scoop)) in 600 μL sterile saline solution. All of the prepared samples in the bead tubes were then homogenized using a Bullet blender® STORM bead mill homogenizer (NEXT ADVANCE) at speed 12 for 5 minutes to generate tissue homogenate. Fifty-microliter tissue homogenates were transferred into 1.5 microtubes (GeneMate) and stored at −20° C. to −80° C. for quantitative real-time PCR (qPCR). The remaining tissue homogenates were clarified using an Eppendorf centrifuge 5424R at 13,000 rpm for 15 minutes at 4° C. All of the supernatants (Tissue lysates) were transferred into new microtubes and stored at −20° C. to −80° C. until used for IDS, GAG and protein assays.

Iduronate sulfatase assay. IDS enzyme activity was measured in tissue lysates using 4-methylumbelliferyl-α-L-iduronide-2-sulphate disodium (4-MU-αldoA-2S: Toronto Research Chemical Incorporation, Cat. #M334715) as substrate in a two-step assay. Tissue lysates were mixed with 1.25 mM MU-αldoA-2S (in 0.1 M sodium acetate buffer pH 5.0+10 mM lead acetate+0.02% sodium azide) and incubated at 37° C. for 1.5 hours. The first-step reaction was terminated with PiCi buffer to stop IDS enzyme activity (0.2 M Na₂HOP₄/0.1 M citric-acid buffer, pH 4.5+0.02% Na-azide). A final concentration of 1 μg/ml Iduronidase (IDUA: R&D Systems, Cat. #4119-GH-010) was added into the tubes to start the second-step reaction. The tubes were incubated overnight at 37° C. to cleave 4-MU-IdoA into 4-MU. The second-step reaction was terminated by adding 200 μl stop buffer (0.5 M Na₂CO_{3+0.5} M NaHCO₃, 0.025% Triton X-100, pH 10.7). The tubes were centrifuged using an Eppendorf centrifuge 5415D at 13,000 rpm for 1 minute. Supernatants were transferred into a roundbottom black 96-well plate and fluorescence measured at excitation 365 nm and emission 450 nm, 75 sensitivity using a Synergy MX plate reader and spectrophotometer (Bio Tek) with Gen5 plate reader program. Enzyme activity is expressed in nmol/hr/ml plasma for plasma samples and in nmol/hr/mg protein for tissue extracts. Protein was determined using the Pierce™ 660 nm Protein Assay Reagent with BSA as a standard (CAT. #23208; Thermo Scientific, MN).

Glycosaminoglycan assay. Tissue lysates were incubated overnight with Proteinase K, DNase1 and RNase as previously described (Wolf et al. 2011), then GAG contents assessed using the Blyscan™ Sulfated Glycosaminoglycan Assay kit (biocolor life science assays, Accurate Chemical). Blyscan glycosaminoglycan standard 100 μg/mL (CAT. #CLRB 1010: Accurate Chemical, NY) was used to make a daily standard curve. Absorbance was measured at 656 nm using a Synergy MX plate reader and spectrophotometer (Bio Tek) with the Gen5 plate reader program. The blank value was subtracted from all readouts. Tissue GAG content is reported in μg GAG per mg protein, and urine GAG content is reported as ug GAG per mg creatinine. Urine creatinine was measured using the Creatinine Assay Kit (Sigma-Aldrich®) according to the manufacturer's instructions Quantitative real-time PCR (qPCR) for IDS vector sequences. Tissue homogenates were mixed with 300 μILcell lysis buffer (5 Prime) and with 100 μg of proteinase K, gently rocking overnight at 55° C. DNA was isolated from the sample by phenol/chloroform extraction. Reaction mixtures of 20 μl contained 60 ng of DNA template, 10 μl of FastStart Taqman Probe Master mix (Roche), 200 nM each of forward and reverse primers and 100 nM of Probe. A C1000 Touch™ Thermo Cycler (BIO-RAD) equipped with CFX manager software version 3.1 was used for qPCR reaction. The PCR conditions were: 95° C. for 10 minutes, followed by 40 cycles of 95° C. for 15 seconds and 60° C. for 1 minute. IDS primers used were forward primer: 5′-GCCAAAAATTATGGGGACAT-3′ (SEQ ID NO:1); IDS reverse primer: 5′-ATTCCAACACACTATTGCAATG-3 (SEQ ID NO:2)′; IDS probe: 6FAM-ATGAAGCCCCTT GAGCATCTGACTTCT-TAMRA (SEQ ID NO:3)

To prepare the standard, pENN.AAV.CB7.hIDS was linearized by digestion with Sall restriction enzyme (New England BioLab Inc.). The linearized plasmid DNA was then purified using the 5Prime DNA Extraction kit. The plasmid DNA concentration was measured using a NanoDrop 1000 spectrophotometer (Thermo Scientific) with NanoDrop 1000 3.7.0 program. The purified linearized plasmid DNA was then diluted to prepare the qPCR standard curve. UltraPure™ distilled water (Invitrogen) was used as negative control. A 10-fold dilution series of linearized plasmid was used to generate a standard curve with a range of 1 to 10⁸ plasmid copies per assay in duplicate with amplification efficiencies between 90%-110% and R² of 0.96-0.98. Vector copy was calculated based on a daily standard curve and expressed as vector genomes per cellular genome equivalent (vg/ge).

Neurocognitive testing in the Barnes maze. The Barnes maze (Barnes 1979) is a circular platform measuring approximately 4 feet in diameter and is elevated approximately 4 feet from the floor with 40 holes spaced equally around the perimeter. All of the holes are blocked except for only one hole that is open for the mouse to escape the platform. Different visual cues were attached to each of the 4 walls for the mouse to use as spatial navigators. At 6 months of age, test mice were placed in the middle of the platform with an opaque funnel covering the mouse. The cover was lifted, releasing the mouse and exposing it to bright light. The animal is expected to complete the task by escaping the platform using the one open hole within 3 minutes. Each mouse was subjected to 4 trials per day for a total of 6 days. The time that the mouse required to escape the platform in each trial was recorded and the average was calculated for each day in each group.

Statistical analysis. Data are reported as mean±S.E. Statistical analyses were performed using Prism 6. Two-way ANOVA with Tukey's post-test was used to evaluate the significance of differences among test groups for IDS assay, GAG assay, and neurobehavioral assay with P value less than 0.05 considered significant. A two-tail t-test on Microsoft Excel was used to evaluate differences in IDS activities between the left and the right hemispheres of microdissected brain.

### Results

Comparison of vector constructs and route of administration to achieve IDS expression in the CNS. A pilot study was conducted to compare several AAV vector constructs and to find a suitable route of administration resulting in IDS expression in the CNS. NOD. SCID mice were used for this study to circumvent the potential complication of an anti-IDUA immune response. The 4 vectors (AAV9.hIDS, AAV9.hIDSco, AAV9.hIDS-hSUMF1, and AAV9.hIDSco-hSUMF1co) were delivered by intrathecal (IT) administration. SUMF1 encodes an enzyme which post-translationally modifies an amino acid in sulfatases, including IDS, resulting in conversion into catalytically active forms. The addition of SUMF1 to some of the vectors was to determine if SUMF1 activity is rate-limiting in producing active IDS protein when IDS is overexpressed. Five untreated IDS+NOD. SCID mice were used as a control group. Six weeks post injection, the mice were euthanized, harvesting and microdissecting the brain into different portions. Parallel studies in MPS I have demonstrated supraphysiological activities of IDUA in the CNS post-IT injection of AAV9 vector encoding hIDUA (Belur et al. 2014). It was expected to see high levels of IDS, exceeding the endogenous level in IDS+NOD.SCID mice administered AAV9.hIDS vector. Surprisingly, no significant increase of IDS activity was observed in the CNS exceeding the endogenous level observed in uninjected NOD.SCID mice regardless of vector construct (data not shown). AAV9.hIDS was also injected into 2 groups of 3 wild type C57BL/6 (IDS+C57BL/6) mice at 8 weeks of age, one group via IT administration and the other group via intravenous administration (IV). Again no significant increase in the level of IDS activity was observed in the CNS above the endogenous level of untreated controls (data not shown). Thus, neither IT nor IV injection of IDS-encoding AAV vector appeared to be a suitable route of administration.

Another unexpected result from the initial study described above is that while there was undetectable increase in IDS activity in the CNS, plasma IDS activity in both IV and IT treated groups was increased up to approximately 140-fold above the untreated wild type level and persisted for at least 12 weeks post-treatment. For the IT treated animals this result suggests that AAV vector was distributed to the peripheral circulation after injection into the cerebrospinal fluid. The presence of sustained enzyme activity for at least 12 weeks post-injection (either IT or IV) also suggests that hIDS is presumably nonimmunogenic for C57BL/6 mice.

The same 4 vector constructs (AAV9.hIDS, AAV9.hIDSco, AAV9.hIDS-hSUMF1, and AAV9.hIDSco-hSUMF1co) were administered to immunocompetent MPS II mice by intracerebroventricular injection, a procedure that supports a much higher level of transduction in the CNS than IT injection. Immunosuppression of the MPS II test animals was not necessary since it was found that expression of human IDS does not elicit an immune response in C57BL/6 mice. An additional group of MPS II mice was injected ICV with a combination of 2 vectors: AAV9.hIDS and AAV9-hSUMF1 at a 1:1 ratio (AAV9.hIDS+AAV9.hSUMF1; at a dose of 5×10¹⁰ vg total) to determine if there would be additional IDS activity under conditions in which SUMF1 expression is optimized as compared to driving expression from an IRES. Untreated wild type littermates were used as controls. Six weeks post injection the animals were euthanized, organs were harvested, and brains were microdissected to determine IDS activity. Animals injected with AAV9.hIDS, AAV9.hIDS-hSUMF1, or AAV9.hIDS+AAV9.hSUMF1 showed levels of IDS activity approximately 10% to 40% of the wild type level in most portions of the brain. IDS activity was undetectable in all areas of the brain in MPS II mice. Animals injected with codon-optimized vector constructs showed mostly less than 10% of the wild type level. There was no significant difference between AAV9.hIDS injected animals and animals injected with AAV9.hIDS plus hSUMF1. Thus in our hands co-delivery of hSUMF1 either on the same vector or on a separate vector did not enhance the level of IDS activity assessed. Vector AAV9.hIDS was subsequently used for more extensive efficacy studies in ICV administered MPS II mice as described below, as neither the addition of SUMF1 nor the codon-optimization algorithm used resulted in increased IDS activity as compared to the native hIDS cDNA sequence.

Prevention of CNS and peripheral lysosomal disease by Intracerebroventricular administration of AAV9.hIDS vector. A dose of 5.6×10¹⁰ AAV9. hIDS vg was infused into eight-week old MPS II mice by ICV injection to achieve widespread CNS distribution of the vector through the cerebrospinal fluid (CSF). As in the pilot study, plasma IDS activities were observed up to 160-fold higher than wild-type in this larger cohort of ICV-treated MPS II animals, and this expression persisted throughout the experiment (28 weeks post injection). Urine was collected at the end of the study (week 40 post-injection) to evaluate the effect of long-term IDS expression on GAG excretion in the treated animals compared to wild type and untreated MPS II mice. Urine GAG was significantly elevated in MPS II animals when compared to wild-type littermates (p<0.05). The treated animals demonstrated a significant reduction in urine GAG content (p<0.05) when compared to untreated littermates and were normalized when compared to the wild type level (p>0.05).

At 10 months of age (40 weeks post injection) all mice were euthanized and organs were harvested for analysis. IDS activity was undetectable in all areas of the brain and spinal cord of untreated MPS II mice. AAV9.hIDS injected animals had IDS activity in all regions of the brain at approximately 9% to 28% of wild type, 53% in olfactory bulb and 7% in the spinal cord. Although the vector was infused into the right ventricle of the brain, we did not observe a significant difference in IDS activity between the left and the right hemispheres (p>0.05). Unlike the CNS, supraphysiological levels of enzyme activity were observed in all tested peripheral organs such as heart, liver, spleen and kidney (11-, 166-, 5—, and 3-fold, respectively), except in the lung where it was observed to be 34% of wild type.

DNA was isolated from the same tissue homogenates and evaluated for vector distribution by qPCR. Consistent with Wolf et al. (Wolf et al. 2011), ICV infusion of AAV vector resulted in global distribution of vector in the CNS and in all of the tested peripheral tissues. The highest vector copy number was in the right hippocampus (49 vg/ge), while similar vector copies were observed between the left and the right hemisphere for all regions of the brain. Unlike the CNS, relatively low copy numbers were detected in most tested peripheral tissues include heart, lung, spleen and kidney of the treated animals (less than 0.6 vg/ge), while high vector copy number was observed in the liver (44 vg/ge). This suggested that enzyme produced by the liver was released into the circulation where the tested peripheral organs took up the circulating enzyme (i.e. metabolic cross-correction).

The global distribution and expression of IDS had a significant effect on accumulation of lysosomal storage materials. Elevation of lysosomal GAG content was observed in the CNS of untreated MPS II mice when compared to wild type littermates (p<0.0001). Even though there was only 10% to 40% of wild type IDS level in the CNS, GAG content in the CNS was normalized when compared to wild type (p<0.01). Similar to the CNS, significant elevation of lysosomal GAG content was observed in all tested peripheral organs of the untreated MPS II animals when compared to wild type (p<0.01). In contrast, significantly decreased GAG levels were observed in all tested peripheral tissues of the treated mice when compared to the untreated group (p<0.01). Statistical analysis showed no significant difference in GAG content between wild type animals and the treated group (p>0.05), which indicates that GAG content was normalized in the treated group.

The body weights of all mice were measured before sacrificed, and organs were weighed after the animals were perfused with 1×PBS, calculating the percentage of organ weight to body weight immediately post-sacrifice. We observed no significant difference in the size of heart, lung, spleen, and kidney amongst all groups. However, the liver of untreated MPS II mice was 20% larger than that of wild type animals (6.2% and 5.2% of total body weight, respectively; p<0.001). In contrast, the liver of the treated MPS II mice was 68% smaller than the untreated group (4.2% and 6.2% of total body weight, respectively; p<0.0001). This result shows that normalization of GAG content in the liver in turn prevented hepatomegaly in the treated mice.

Sustained expression of IDS in the CNS leads to prevention of neurocognitive deficit in MPS II mice. At 6 months of age (4 months post-treatment), untreated MPS II mice, AAV9. hIDS treated MPS II mice and control normal littermates were evaluated for neurocognitive function in the Barnes maze, a test for spatial navigation and memory. The animals were subjected to a series of 3-minute trials, 4 trials per day for a course of 6 days. Wild type littermates showed reduced latency to escape (30 s) on day 6, while untreated MPS II mice exhibited a significant deficit in learning this task (latency to escape reduced only to 71 seconds; p≤0.05 vs normal littermates). In contrast, the AAV9.hIDS treated mice showed a marked reduction in latency to escape (25s), significantly outperforming the untreated MPS II mice (p≤0.01) on day 5 and day 6. In addition, there was no significant difference between the treated animals and wild type littermates (p>0.05). We conclude that sustained expression of IDS in the CNS prevents the emergence of neurocognitive deficits in MPS II mice when treated at a young age.

### DISCUSSION

In this study, AAV9.hIDS was administered using a strong promoter to the CNS of MPS II mice. Levels of IDS activity in the CNS were only 7% to 28% of wild type. In contrast, levels of IDS activity in the circulation and in tested peripheral organs were at least 2-fold and up to 170-fold higher than wild type levels. It was also observed that sustained IDS expression leads to global normalization of GAG content. Finally, it was observed that sustained levels of IDS activity had a profound effect on preventing neurologic deterioration.

No significant increase in IDS activity was observed in the CNS above the endogenous level in IV or IT treated IDS+ mice regardless of vector construct, route of administration, or mouse strain, even though IDS was expressed under regulation of the strong CB7 promoter. Similar results were also observed when AAV9.hIDS was infused into MPS II mice via ICV injection; although it was observed IDS activity in the CNS of these mice above baseline, it was lower than wild type levels at 40 weeks post-treatment. Similar results were reported from Motas et al. (Motas et al. 2016) and Hinderer et al (Hinderer et al. 2016), in which they observed approximately 20% to 40% of wild type levels in the CNS. These limited levels of IDS activity are in stark contrast with the levels of IDUA activity observed in the CNS of MPS I mice after ICV injection of AAV9-IDUA vector, in which 100- to 1000-fold higher than wild type levels of IDUA activity are observed (Belur et al. 2014). Supraphysiological levels of IDS (>1000 nmol/hr/mg) were also observed in the liver of ICV administered animals in our study at a similar vector copy number that yielded 10- to 100-fold less IDS expression (10 to 100 nmol/hr/mg) in the CNS. Highly relevant to the goal of CNS-directed gene therapy for MPS II, therefore is this question: What is it that limits expression of IDS in the brain after highly efficient AAV mediated IDS gene delivery?

One possibility is that SUMF1 activity might be rate limiting for the generation of active IDS in the brain. SUMF1 is required for the post-translational activation of lysosomal sulfatases, including IDS (Sabourdy et al. 2015). hIDS enzyme clearly was activated in tissues of the MPS II mouse, presumably by mouse SUMF1, but this process could be rate limiting in the brain, if AAV-encoded hIDS protein is expressed in a large quantity but only a limited amount of hIDS becomes activated. For example, Fraldi et al (Fraldi et al. 2007) demonstrated that N-sulfoglucosamine sulfohydrolase activities were increased when the enzyme was co-expressed with SUMF1 in their MPS IIIA studies. Anticipating a potential limitation of SUMF1, we co-transduced hIDS and hSUMF1 either on the same construct or on 2 separate vectors, but we found no significant increase in hIDS activity compared to delivery of hIDS alone. It was concluded that hIDS activity was not enhanced by co-delivery with hSUMF1 in these experiments. Further studies evaluating SUMF1 mediated activity of IDS in the CNS are nonetheless warranted.

Another possibility is that the CB7 promoter might be limiting when compared to the endogenous IDS promoter. To further investigate this possibility, IDS activity (nmol/h/mg protein=unit) per vector copy was calculated for each tissue and compared to IDS activity per endogenous copy in male mice. IDS activities in the CNS of the treated mice were observed between 2 and 32 units per vector copy compared to wild type male mice, which express an average of 200 units per genome equivalent (approximately only 1% to 31% of wild type level). From this one might conclude that the CB7 promoter is not as robust as the endogenous IDS promoter in the brain. However, the lower level of vector mediated IDS expression per vector copy vs endogenous expression most likely results from the presence of excess AAV vector in the brain post-injection that does not become intracellularized and expressed. Nonetheless, the MPS II results contrast greatly with the results from the present MPS I studies in which levels of IDUA activity at approximately 10- to 100-fold higher than wild type were observed in the CNS of mice administered AAV-hIDUA intrathecally. (Belur et al. 2014), while heterozygous MPS I animals express approximately 6 units per genome equivalent in the brain (Ou et al. 2014). Therefore it is feasible to achieve or exceed wild type IDUA levels in the CNS of MPS I mice. The promoter that we used in our MPS II study was similar but not identical to the promoter used in the MPS I studies (Wolf et al. 2011; Belur et al. 2014), and we cannot exclude the possibility that relative promoter strength may have contributed to the observed differences in outcome.

Surprisingly, supranormal levels of enzyme activity were observed in all tested peripheral organs of the treated animals. The highest levels of IDS activity (164-fold wild type) were observed in the liver, which correlated with the highest number of vector copies found (48 vc/ge). In contrast, the tested peripheral organs (other than liver) contained low levels of vector, but higher than wild type levels of IDS activity. The exceptionally high levels of IDS in the liver lead to sustained high levels of IDS activity (up to 172-fold higher than wild type) in the circulation. Similar results have been reported in a MPS IIIA study and several MPS I studies using different types of vectors (Aronovich et al. 2009; Osborn et al. 2011; Haurigot et al. 2013), along with 2 studies in MPS II mice (Motas; Hinderer). These results suggest that in our study liver acts as an enzyme factory that produces an enormous amount of IDS and releases it into the circulatory system where it is subsequently taken up by the other organs in the periphery. Even though we observed higher than wild type levels of IDS enzyme activity in heart, spleen and kidney in the treated mice, the vector levels in these organs was unexpectedly low (less than 0.6 vc/ge). This low transduction rate suggests that these organs took up circulating IDS leading to metabolic cross-correction with higher levels of IDS than wild type.

Polito et al. showed no IDS activity in the CNS after a single IV injection of AAV2/5 CMVhIDS. However, they observed partial correction of GAG content in the CNS (Polito and Cosma 2009). They speculated that only a fraction of high level circulating hIDS penetrated the BBB into the CNS with subsequent partial correction of GAGs. Similarly, even though there were exceptionally high levels of IDS in the circulation of AAV9. hIDS treated mice, we observed only subphysiological activities of the enzyme in the CNS. This finding indicates that only insignificant amounts of the circulating enzyme if any were able to penetrate the BBB into the CNS. Therefore the level of IDS observed in the brain most likely relied on expression of the vector construct inside the CNS rather than from the circulating enzyme. Further investigation is needed to achieve IDS levels comparable to or higher the wild type in the CNS.

Sustained levels of enzyme expression after direct injection into the CNS have been shown to have profound effects on GAG reduction in several MPS studies whether or not wild type levels of those enzymes were achieved (Belur et al. 2014; Wolf et al. 2011; Motas et al. 2016; Haurigot et al. 2013) (Hinderer et al 2016). Similarly, although we observed only subphysiological levels of IDS in the CNS after ICV injection of AAV9.hIDS, there was a significant effect of the enzyme on GAG reduction that was greater than expected. Desnick et al. and Polito et al. speculated that only less than 5% of the wild type level of lysosomal enzyme is needed to correct a GAG storage defect (Desnick and Schuchman 2012; Polito and Cosma 2009). The present results are consistent with this speculation. After AAV9.hIDS treatment, it was found that all areas of the brain and even spinal cord (the organ with the lowest enzyme measured; 10.7 nmol/h/mg protein; 7.4% of wild type showed significantly lower GAG content than untreated MPS II mice. Statistical analysis revealed striking results as GAG content in the CNS of the treated mice was normalized when compared to wild type. It was also observed normalization of GAG content in the urine and in heart, lung, liver, spleen and kidney of the treated animals. These results indicate a sustained effect of IDS expression on correction of GAG content.

Roberts et al. demonstrated a direct relationship between GAG accumulation and liver size when injecting rodamine B, a GAG synthesis inhibitor, into MPS IIIA mice. They found that GAG content was decreased in the liver, leading to normalization of liver size (Roberts et al. 2006). Motas et al. observed a preventive effect on hepatomegaly after ICV administration of AAV9-hIDS into MPS II mice (Motas et al. 2016). Similarly, it was also observed that the weight of the liver in treated mice was normalized after ICV injection of AAV9.hIDS. This result indicates a profound effect of sustained IDS expression leading to normalization of GAG content in the liver, which in turn prevents hepatomegaly in the treated mice.

Several MPS studies have demonstrated prevention of neurologic deficits after AAV-mediated gene transfer in mice (Fu et al. 2011; Wolf et al. 2011; Motas et al. 2016). It was observed neurocognitive deficiency in untreated MPS II mice at 6 months of age. We also observed that sustained IDS expression in the CNS in prevented the emergence of neurocognitive dysfunction in MPS II mice after ICV infusion of AAV9.hIDS. Several studies have shown that the hippocampus is associated with neurocognition in rodents (Seeger et al. 2004; Paylor et al. 2001; Miyakawa et al. 2001). However, the possibility that physical impairment such as vision, olfactory sense, or motor neuron defects could also affect the results in the Barnes maze could not be excluded, as rodents require all of the aforementioned physical capabilities in order to perform the required task in this test. (Harrison et al. 2006). Additional behavioral analyses would support our observation that neurocognitive deficit plays a pivotal role in learning impairment of untreated MPS II mice and its prevention by AAV mediated IDS gene transfer.

In conclusion, the present results show the benefit of direct AAV9-mediated hIDS gene transfer to the CNS. However, the limited level of AAV-mediated IDS expression achieved in the CNS in comparison with other tissues such as liver, and in comparison with the expression of other therapeutic genes introduced into the CNS by AAV mediated transduction, such as IDUA (Belur et al. 2014; Wolf et al. 2011), was surprising. Nonetheless, the MPS II data indicate that direct injection of AAV9-hIDS vector into the CNS resulted in efficient gene transfer that is key to treatment of MPS II and prevention neurocognitive deficits. We found that the AAV9.hIDS vector was capable of not only crossing the BBB resulting in global transduction of the vector both inside and outside of the CNS, but also providing long-term expression of IDS enzyme systemically. Sustained IDS expression corrected the accumulation of GAG in liver and subsequently prevented the emergence of hepatomegaly. In addition, our results reinforce the importance of sustained IDS expression in the CNS in preventing the emergence of neurologic deficits when animals are treated at a young age.

### Example XI

Mucopolysaccharidosis type I (MPS I) is an inherited autosomal recessive metabolic disease caused by deficiency of α-L-iduronidase (IDUA), resulting in accumulation of heparin and dermatan sulfate glycosaminoglycans (GAGs). Individuals with the most severe form of the disease (Hurler syndrome) suffer from neurodegeneration, mental retardation, and death by age 10. Current treatments for this disease include allogeneic hematopoietic stem cell transplantation (HSCT) and enzyme replacement therapy (ERT). However, these treatments are insufficiently effective in addressing CNS manifestations of the disease.

The goal is to improve therapy for severe MPS I by supplementing current ERT and HSCT with IDUA gene transfer to the CNS, thereby preventing neurological manifestations of the disease. In this study, the ability of intravenously administered AAV serotypes 9 and rh10 (AAV9 and AAVrh10) to cross the blood brain barrier for delivery and expression of the IDUA gene in the CNS was tested. 4-5 month old adult MPS I animals were infused intravenously via the tail vein with either an AAV9 or AAVrh10 vector encoding the human IDUA gene. Blood and urine samples were collected on a weekly basis until the animals were sacrificed at 10 weeks post-injection. Plasma IDUA activities in treated animals were close to 1000-fold higher than that of heterozygote controls at 3 weeks post-injection. Brains, spinal cords, and peripheral organs were analyzed for IDUA activity, clearance of GAG accumulation, and IDUA immunofluorescence of tissue sections. Treated animals demonstrated widespread restoration of IDUA enzyme activity in all organs including the CNS. These data demonstrate the effectiveness of systemic AAV9 and AAVrh 10 vector infusion in counteracting CNS manifestations of MPSI.

### Example XII

Gene transfer offers enormous potential for therapy of the mucopolysaccharidoses. Studies have focused on achieving high-level expression of alpha-L-iduronidase (IDUA) in the CNS of MPS I mice, where it was observed enzyme up to 1000-fold greater than wild-type (WT) in the brain after intracerebroventricular (ICV) infusion of AAV9 transducing the human IDUA gene. Intrathecal (IT) infusion of AAV9 vector also resulted in high-level IDUA expression (10- to 100-times that of wild-type) throughout the brain. All routes of administration normalized glycosaminoglycan levels in all areas of the brain and prevented the emergence of neurocognitive deficiency at 5 months of age as assessed in the Barnes maze. WT mice expressed much higher levels of endogenous iduronate sulfatase (IDS) than IDUA in the brain, and in animals infused IT with AAV9 transducing the human IDS gene, the level of IDS in the brain was indistinguishable from WT. After ICV infusion of AAV9-IDS vector in MPS II mice there was sufficient expression of IDS to reduce GAG accumulation and prevent the emergence of neurocognitive dysfunction, but the level of IDS never achieved that of WT in the brain. Extremely high levels of enzyme (1000-times that of WT) were detected in the plasma of MPS I animals infused intravenously with AAV9 vector transducing the IDUA gene. Staining for IDUA showed a high frequency of transduced cells in the liver, with a much smaller number of transduced cells observed in the parenchyma and vessels of the brain. Overall, these results provide a comprehensive assessment of the relative effectiveness of different routes of AAV vector administration associated with different degrees of invasiveness in two mouse models of mucopolysaccharidosis.

### Example XIII

Hunter Syndrome (Mucopolysaccharidosis type II; MPS II) is an X-linked recessive inherited lysosomal disease caused by deficiency of iduronate-2-sulfatase (IDS) and accumulation of glycosaminoglycans (GAGs) in tissues, resulting in skeletal dysplasias, hepatosplenomegaly, cardiopulmonary obstruction, and neurologic deterioration. Patient standard of care is enzyme replacement therapy (ERT) although ERT is not associated with neurologic improvement. In a mouse model of IDS deficiency, intracerebroventricular (ICV) administration of AAV9.hIDS into young 8-week old mice resulted in corrective levels of hIDS enzyme activity, reduction of GAG storage to near WT-levels and prevention of neurocognitive dysfunction, compared to IDS deficient control littermates. Since the emergence of neurologic manifestations could be prevented in young adults, it was hypothesized that older adult MPS II animals treated at 4 months of age by ICV administration of AAV9.hIDS would recover neurobehavioral function and show corrected levels of IDS enzyme activity and GAG storage. By 4 weeks post-ICV injection, IDS enzyme activity in the circulation was 1000-times that of WT-levels (305+/−85nmol/hr/ml compared to 0.39+/−0.04nmol/hr/ml). At 36 weeks of age, the treated animals were tested for neurocognitive function in the Barnes maze. Performance of the treated animals was indistinguishable from that of unaffected littermates and significantly improved compared to untreated MPS II mice. Cognitive function that is lost by 4 months of age can thus be restored in MPS II mice by delivery of AAV9 encoding IDS to the cerebrospinal fluid. The exciting implication of these results is the prospect that human MPS II may be treatable after the development of neurologic manifestations by AAV mediated IDS gene transfer to the CNS.

### Example XIV

Mucopolysaccharidosis type I (MPS I) is a progressive, multisystemic disease caused by deficiency of α-L-iduronidase (IDUA). Current treatments are ineffective against CNS disease. Our goal is to improve therapy for severe MPS I by supplementing current treatments with IDUA gene transfer to the CNS. AAV9-IDUA vector was delivered to the brains of 6-8 week old MPS I mice using different routes of administration, resulting in supraphysiological levels of IDUA enzyme and prevention of neurologic disease. However, since MPS I is a relentlessly progressive and fatal disease, our goal in this study was to treat MPS I mice that had already developed significant pre-existing disease, and to ascertain the therapeutic effects on metabolic and neurocognitive deficits. MPS I animals were immunotolerized at birth with IDUA (Aldurazyme), and then administered AAV9-IDUA vector by intracerebroventricular infusion at 6 months of age, at which point untreated MPS I animals have already developed significant neurologic deficit. Plasma IDUA activities in the treated animals were 1000-fold higher than WT controls starting at 6 weeks post-treatment. At 10 months of age the treated animals, along with age-matched WT and IDUA-deficient controls, were subjected to neurocognitive testing using the Barnes maze. As previously demonstrated, untreated MPS I mice displayed a significant neurocognitive deficit in comparison with unaffected littermates. Remarkably, MPS I mice treated with AAV-IDUA post-symptomatically exhibited behavior similar to that of the WT controls, demonstrating correction of the neurocognitive deficit found in untreated animals at 6 months of age. Treated animals sacrificed at 12 months demonstrated widespread restoration of IDUA enzyme activity in the brain, spinal cord and liver. These results demonstrate effectiveness of AAV9-IDUA in the recovery of MPS I mice from CNS disease accumulated to a significant load, with potential application to the treatment of human MPS I after development of neurologic manifestations.

### Example XV

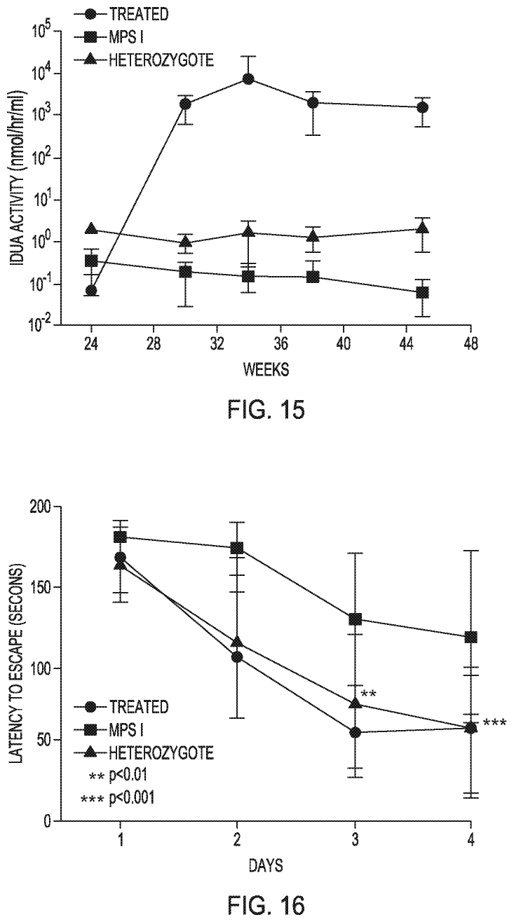

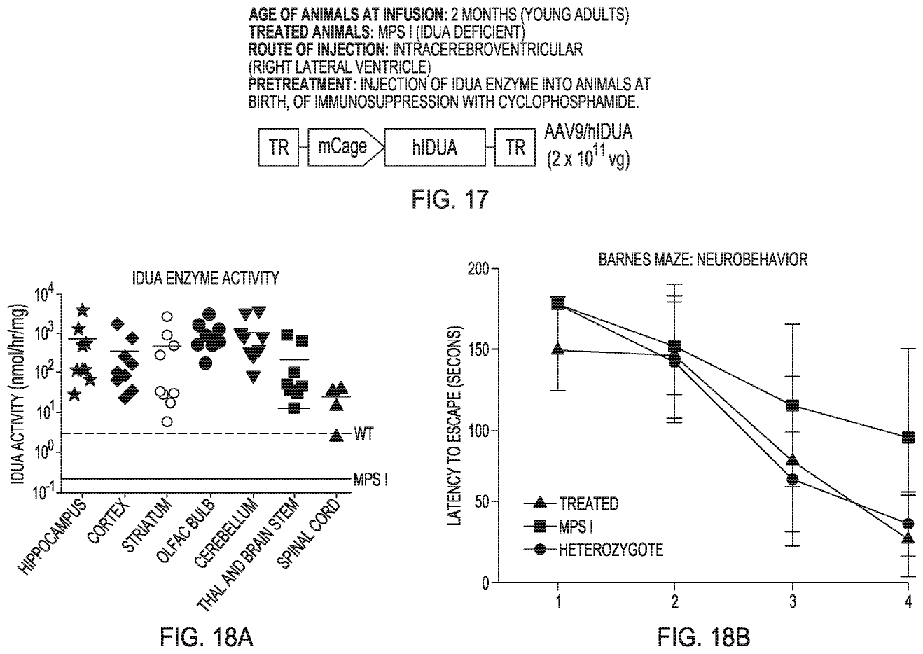

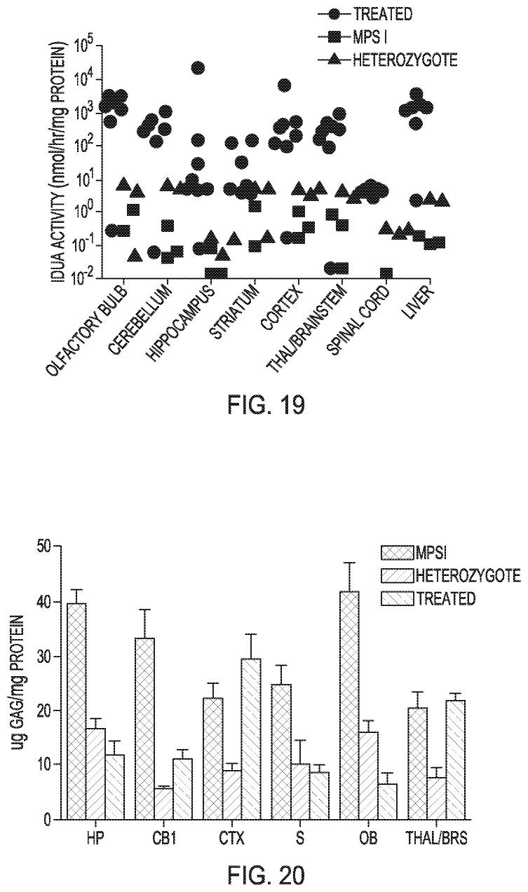

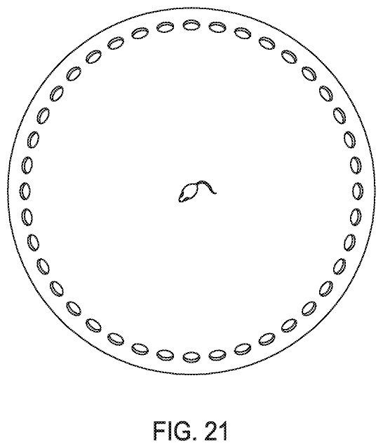

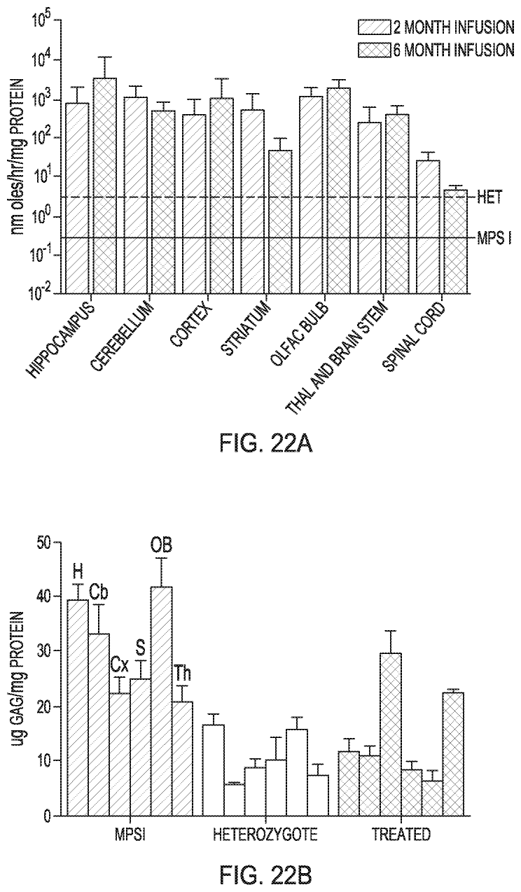

MPSI is caused by the absence of IDUA which catalyzes the degradation of GAGs. Lack of IDUA causes accumulation of GAGs and leads to growth delay, hepatosplenomegaly, cardiopulmonary disease and skeletal dysplasia, as well as neurological impairment. Current treatments (HSCT and ERT) do not adequately address this debilitating neurologic disease as HSCT results in only particle correction of neurological impairment and lysosomal enzymes to not cross the blood brain barrier. . . . To test the efficacy of AAV-IDUA in older (adult) mice with established disease, newborn mice were immunotolerized with IDUA at birth and at 6 months of age infused ICV with AAV9-IDUA (FIG. 17). Specifically, MPSI mice were immunotolerized with 5 doses of Laronidase (Aldurazyme) administered weekly, starting at birth, followed by infusion of AAV9-IDUA vector at 6 months of age. IDUA enzyme activity in plasma and brain was 100 fold higher than wild type at 6 months (FIGS. 15, 18 and 19), and a reduction in GAG levels (FIG. 6). Moreover, treated mice had reduced neurocognitive deficit (FIG. 16).

In summary, in treated mice, IDUA enzyme activity was high in all areas of the brain measured, there were reduced levels of GAG, and improved neurocognitive function. Thus, gene therapy is useful in established MPSI disease.

### REFERENCES

Al-Ghananeem et al., AAPS Pharm. Sci. Tech., 3: E5 (2002).
Aronovich et al., J. Gene Med., 9:403 (2007).
Aronovich et al., Mol. Ther., 17:1136 (2009).
Aronovich, Mol. Genet. Metab., 114:83 (2015).
Bagger et al., Eur. J. Pharm. Sci., 21:235-242 (2004b).
Bagger et al., Int. J. Pharm., 269:311-322 (2004a).
Baker et al., Exp. Brain Res., 63:461 (1986).
Balin et al., J. Comp. Neurol., 251:260-280 (1986).
Banks et al., J. Drug Target, 17:91-97 (2009).
Banks et al., J. Pharmacol. Exp. Ther., 309:469 (2004).
Banks, Biopolymers, 90:589 (2008).
Barakat et al., J. Pharm. Pharmacol., 58:63 (2006).
Barbier et al., Mol. Genet. Metab., 110:303 (2013).
Barnes, J. Comp. Physiol. Psychol., 93:74 (1979).
Baumgartner et al., Neuron., 58:639 (2008).
Belur et al., Mol. Ther., 22: S236 (2014).
Benedict et al., Neuroendocrinology, 86:136 (2007b).
Benedict et al., Neuropsychopharmacology, 32:239 (2007a).
Benedict et al., Psychoneuroendocrinology, 29:1326 (2004).
Bennett et al., Sci. Transl. Med., 4: 120ra15 (2012).
Bjoraker et al., J. Dev. Behav. Ped., 27:290 (2006).
Blits et al., J. Neuros. Methods, 185:257 (2010).
Born et al., Nat. Neurosci., 5:514 (2002).
Boulton et al., Am. J. Physiol., 276: R818 (1999).
Boulton et al., Neuropathol. Appl. Neurobiol., 22:325 (1996).
Bradbury et al., Am. J. Physiol., 240: F329 (1981).
   Bradbury et al., J. Physiol., 339:519 (1983).
   Brady, Ann. Rev. Med., 57:283 (2006).
   Broadwell et al., J. Comp. Neurol., 242:632 (1985).
   Broekman et al., Neuroscience, 138:501 (2006).
   Buck, In: Kandel ER, Schwartz JH, Jessell™, editors. Principles of neural science. 4th edition. New
   York: McGraw-Hill Companies. pp. 625-652 (2000).
   Buxer et al., J. Neurochem., 56:1012 (1991).
   Cai et al., Sichuan Da Xue Xue Bao Yi Xue Ban, 39:438 (2008).
   Campos et al., Meta. Brain Dis., 27:121 (2012).
   Capsoni et al., Proc. Natl. Acad. Sci. USA, 99:12432 (2002).
   Carare et al., Neuropathol. Appl. Neurobiol., 34:131 (2008).
   Cauna, In: Proctor DF, Andersen I, editors. Amsterdam: Elsevier Biomedical Press. pp. 45-69 (1982).
   Charlton et al., Int. J. Pharm., 338:94 (2007b).
   Charlton et al., J. Drug Target, 15:370 (2007a).
   Charlton et al., Pharm. Res., 25:1531 (2008).
   Chen et al., J. Alzheimers Dis., 1:35 (1998).
   Chen et al., J. Pharm. Sci., 95:1364 (2006).
   Chen, Mol. Ther., 8:495 (2003).
   Chow et al., J. Pharm. Sci., 88:754 (1999).
   Chow et al., J. Pharm. Sci., 90:1729 (2001).
   Clerico et al., In: Doty RL, editor. Handbook of olfaction and gustation. 2nd edition. New York: Marcel
   Dekker, Inc. pp. 1-16 (2003).\Campos et al., Meta. Brain Dis., 27:121 (2012).
   Costantino et al., Int. J. Pharm., 337:1 (2007).
   Cserr et al., Am. J. Physiol., 240: F319 (1981).
   Cserr et al., Brain Pathol., 2:269 (1992).
   Dahlin et al., Eur. J. Pharm. Sci., 14:75 (2001).
   Danhof et al., American Association of Pharmaceutical Scientists Annual Meeting, Atlanta, GA (2008).
   Danielyan et al., Eur. J. Cell. Biol., 88:315 (2009).
   Davis et al., Clin. Pharmacokinet., 42:1107 (2003).
   de Lorenzo, In: Wolstenholme GEW, Knight J, editors. Taste and smell in vertebrates. London: Churchill. pp. 151-175 (1970).
   De Rosa et al., Proc. Natl. Acad. Sci. USA, 102:3811 (2005).
   DeSesso, Qual. Assur., 2:213 (1993).
   Desmanis et al., Ann. Neurol., 56:68 (2004).
   Desnick et al., Ann. Rev. Genom. Hum. Genet., 13:307 (2012).
   deSouza et al., Eur. Neuropsychopharmacol., 19:53 (2009).
   Dhanda et al., Drug Del. Tech., 5:64 (2005).
   Dhuria et al., J. Pharm. Sci., 98:2501 (2009b).
   Dhuria et al., J. Pharmaceutical Sciences, 99:1654 (2010).
   Dhuria et al., J. Pharmacol. Exp. Ther., 328:312 (2009a).
   Diano et al., J. Clin. Invest., 118:26 (2008).
   Dickson et al., Mol. Gen. Metab., 91:61 (2007).
   Djupesland et al., Laryngoscope, 116:466 (2006).
   Domes et al., Biol. Psychiatry, 61:731 (2007b).
   Domes et al., Biol. Psychiatry, 62:1187 (2007a).
   Dufes et al., Int. J. Pharm., 255:87 (2003).
   Duque et al., Mol. Ther., 17:1187 (2009).
   Einer-Jensen et al., Exp. Brain Res., 130:216 (2000b).
   Einer-Jensen et al., Pharmacol. Toxicol., 87:276 (2000a).
   Einer-Jensen et al., Reproduction, 129:9 (2005).
   Ellinwood et al., Mol. Genet. Metab., 91:239 (2007).
   Fehm et al., J. Clin. Endocrinol. Metab., 86:1144 (2001).
   Field et al., J. Neurocytol., 32:317 (2003).
   Fliedner et al., Endocrinology., 17:2088 (2006).
   Foust et al., Nat. Biotech., 27:5 (2009).
   Foust et al., Nat. Biotechnol., 27:59 (2009).
   Fraldi et al., Hum. Mol. Genet., 16:2693 (2007).
   Francis et al., Brain, 131:3311 (2008).
   Fratantoni et al., Science, 162:570 (1968).
   Frey et al., Drug Delivery, 4:87 (1997).
   Frey II, Drug Del. Tech., 2:46 (2002).
   Fu et al., Mol. Ther., 19:1025 (2011).
   Fuss et al., Eur. J. Neurosci., 22:2649 (2005).
   Gao et al., Biomaterials, 27:3482 (2006).
   Gao et al., Int. J. Pharm., 340:207 (2007a).
   Gao et al., J. Control Release, 121:156 (2007b).
   Gopinath et al., Current Ther. Res., 23:596 (1978).
   Gozes et al., Curr. Alzheimer Res., 4:507 (2007).
   Graff et al., Pharm. Res., 20:1225 (2003).
   Graff et al., Pharm. Res., 22:235 (2005a).
   Graff et al., Pharm. Res., 22:86 (2005b).
   Gray, 15th revised edition (Classic Collectors edition). New York: Bounty Books (1978).
   Grevers et al., Arch. Otorhinolaryngol., 244:55 (1987).
   Groothuis et al., J. Cereb. Blood Flow Metab., 27:43 (2007).
   Gross et al., J. Anat., 135:83 (1982).
   Guastella et al., Biol. Psychiatry, 63:3 (2008).
   Hadaczek et al., Mol. Ther., 14:69 (2006).
   Hallschmid et al., Regul. Pept., 149:79 (2008).
   Han et al., J. Mol. Med., 85:75 (2007).
   Hanson et al., BMC Neurosci., 9: S5 (2008).
   Hanson et al., Drug Del. Tech., 4:66 (2004).
   Hanson et al., In: EPO, editor. Biopharm and HealthPartners Research Foundation (2008).
   Hanson et al., San Diego, CA: Society for Neuroscience (2007).
   Harrison et al., Learn Mem., 13:809 (2006).
   Hartung et al., J. Am. Soc. Gene Ther., 9:866 (2004).
   Hartung et al., Mol. Thera., 9:869 (2004).
   Hashizume et al., Neuro. Oncol., 10:112 (2008).
   Hatterer et al., Blood, 107:806 (2006).
   Haurigot et al., J. Clin. Invest., 123:3254 (2013).
   Herati et al., J. Gene Med., 10:972 (2008).
   Hess et al., Exp. Neuro., 186:134 (2004).
   Hinderer et al., Hum. Gene Ther., 27:906 (2016).
   Hoogerbrugge et al., Lancet, 345:1398 (1995).
   Horvat et al., Eur. J. Pharm. Biopharm., 72:252 (2009).
   Huda et al., Mol. Ther. Methods Clin. Devel., 1:14032 (2014).
   Hussar et al., Chem. Senses, 27:7 (2002).
   Illum, J. Control Release, 87:187 (2003).
   Illum, J. Pharm. Pharmacol., 56:3 (2004).
   Itaya et al., Brain Res., 398:397 (1986).
   Janson et al., Neurosurgery, 74:99 (2014).
   Jansson et al., J. Drug Target, 10:379 (2002).
   Jogani et al., Alzheimer Dis. Assoc. Disord., 22:116 (2008).
   Johnston et al., Cerebrospinal Fluid Res., 1:2 (2004).
   Kakkis et al., Mol. Gen. Met., 83:163 (2004).
   Kandimalla et al., J. Pharm. Sci., 94:613 (2005b).
   Kandimalla et al., Pharm. Res., 22:1121 (2005a).
   Kaufmann et al., EMBO Mol. Med., 5:1642 (2013).
   Kida et al., Neuropathol. Appl. Neurobiol., 19:480 (1993).
   Kirsch et al., J. Neurosci., 25:11489 (2005).
   Klein et al., J. Am. Soc. Gene Ther., 13:517 (2006).
   Koos et al., Neuroreport, 16:1929 (2005).
   Kosfeld et al., Nature, 435:673 (2005).
   Kristensson et al., Acta Neuropathol (Berl), 19:145 (1971).
   Krivit, Springer Seminars in Immunopathology, 26:119 (2004).
   Kumar et al., Curr. Sci., 43:435 (1974).
   Kumar et al., Int. J. Pharm., 358:285 (2008).
   Li et al., Chin. J. Physiol., 48:7 (2005c).
   Li et al., Glia, 52:245 (2005a).
   Li et al., J. Neurocytol., 34:343 (2005b).
   Lock et al., Hum. Gene Ther., 21:1259 (2010).
   Loftus et al., Neuroscience, 139: 1061 (2006).
   Luzzati et al., J. Mol. Biol., 343:199 (2004).
   Mackay-Sim, In: Doty RL, editor. Handbook of olfaction and gustation. 2nd edition. New York: Marcel
   Dekker, Inc. pp. 93-113 (2003).
   Martinez et al., Neuroscience, 157:908 (2008).
   McKinnis et al., J. Pediatr., 129:145 (1996).
   Minn et al., J. Drug Target, 10:285 (2002).
   Miragall et al., J. Comp. Neurol., 341:433 (1994).
   Miyakawa et al., Hippocampus, 11:763 (2001).
   Motas et al., JCI Insight. 1: e86696 (2016).
   Muenzer, J. Pediatrics, 144: S27 (2004).
   Munoz-Rojas et al., Am. J. Med. Gen., 146A: 2538 (2008).
   Nathwani et al., New Engl. J. Med., 371:1994 (2014).
   Neufeld and Muenzer, In A. L. B. C.R. Scriver, W.S. Sly, et al (ed.), McGraw Hill, NY, pg. 3421 (2001).
   Nonaka et al., J. Pharmacol. Exp. Ther., 325:513 (2008).
   Ohlfest et al., Blood, 105:2691 (2005).
   Orchard et al., J. Pediatrics, 151:340 (2007).
   Osborn et al., Mol. Ther., 19:450 (2011).
   Ou et al., Mol. Genet. Metab., 111:116 (2014).
   Owens et al., Diabet. Med., 20:886 (2003).
   Pan et al., Brain Res., 1188:241 (2008).
   Pardridge, NeuroRx, 2:3 (2005).
   Parker et al., Psychoneuroendocrinology, 30:924 (2005).
   Pastores, Exp. Opin. Biol. Ther., 8:1003 (2008).
   Paylor et al., Physiol. Behav., 73:781 (2001).
   Pereira et al., Clin. Chim. Acta: Int. J. Clin. Chem., 387:75 (2008).
   Perl et al., Lancet, 1:1028 (1987).
   Peters et al., Bone Marrow Transpl., 31:229 (2003).
   Polito et al., Am. J. Hum. Genet., 85:296 (2009).
   Pollock et al., J. Anat., 191:337 (1997).
   Raghavan et al., J. Laryngol. Otol., 114:456 (2000).
   Rao et al., Ped. Res., 73:31 (2013).
   Reger et al., J. Alzheimers Dis., 13:323 (2008a).
   Reger et al., Neurobiol. Aging, 27:451 (2006).
   Reger et al., Neurology, 70:440 (2008b).
   Rennels et al., Adv. Neurol., 52:431 (1990).
   Rennels et al., Brain Res., 326:47 (1985).
   Reolon et al., Brain Res., 1076:225 (2006).
   Reolon et al., Cell. Mol. Neurobiol., 29:443 (2009).
   Rimmele et al., J. Neurosci., 29:38 (2009).
   Roberts et al., Pediatr. Res., 60:309 (2006).
   Ross et al., J. Neuroimmunol., 151:66 (2004).
   Ross et al., Neurosci. Lett., 439:30 (2008).
   Sabourdy et al., Orphanet. J. Rare Dis., 10:31 (2015).
   Sakane et al., J. Pharm. Pharmacol., 46:378 (1994).
   Sakane et al., J. Pharm. Pharmacol., 47:379 (1995).
   Sarkar, Pharm. Res., 9:1 (1992).
   Schaefer et al., J. Comp. Neurol., 444:221 (2002).
   Scheibe et al., Arch. Otolaryngol. Head Neck Surg., 134:643 (2008).
   Schley et al., J. Theor. Biol., 238:962 (2006).
   Schulz et al., Endocrinology, 145:2696 (2004).
   Schuster et al., Front Neuroanat., 8:42 (2014).
   Schuster et al., Frontiers in Neuroanatomy, 8:42 (2014).
   Scott et al., Am. J. Hum. Genet., 53:973 (1993).
   Seeger et al., J. Neurosci., 24:10117 (2004).
   Shimizu et al., Int. J. Obes. (Lond), 29:858 (2005).
   Shipley, Brain Res. Bull., 15:129 (1985).
   Skipor et al., Reprod. Biol., 3:143 (2003).
   Steen et al., J. Alzheimers Dis., 7:63 (2005).
   Stefanczyk-Krzymowska et al., Exp. Physiol., 85:801 (2000).
   Takano et al., J. Histochem. Cytochem., 53:611 (2005).
   Tanaka et al., Mol. Genet. Metab., 107:513 (2012).
   Thorne et al., Brain Res., 692:278 (1995).
   Thorne et al., Clin. Pharmacokinet., 40:907 (2001).
   Thorne et al., Neuroscience, 127:481 (2004).
   Thorne et al., Neuroscience, 152:785 (2008).
   Thorne, R G. 2002. The nasal pathways for drug delivery to the central nervous system: Studies with
   protein tracers and therapeutics. Doctoral Dissertation, University of Minnesota.
   Tkac et al., Mag. Res. In Med., 41:649 (1999).
   Tkac et al., Mag. Res. In Med., 50:24 (2003).
   Unger et al., J. Neuropath. Exp. Neuro., 52:460 (1993).
   van den Berg et al., Eur. J. Pharm. Biopharm., 58:131 (2004b).
   Van den Berg et al., J. Drug Target, 11:325 (2003).
   van den Berg et al., J. Neurosci. Methods, 116:99 (2002).
   van den Berg et al., Pharm. Res., 21:799 (2004a).
   Van Diest et al., J. Anat., 128:293 (1979).
   Vellodi et al., J. Inherit. Metab. Dis., 22:638 (1999).
   Vulchanova et al., Mol. Pain, 6:31 (2010).
   Vyas et al., AAPS PharmSciTech., 7: E1 (2006c).
   Vyas et al., Crit. Rev. Ther. Drug Carrier Syst., 23:319 (2006b).
   Vyas et al., J. Drug Target, 13:317 (2005).
   Vyas et al., J. Pharm. Sci., 95:570 (2006a).
   Walter et al., Arch. Histol. Cytol., 69:37 (2006a).
   Walter et al., Neuropathol. Appl. Neurobiol., 32:388 (2006b).
   Wang et al., Cancer Chemother. Pharmacol., 57:97 (2006a).
   Wang et al., Eur. J. Pharm. Biopharm., 70:735 (2008).
   Wang et al., Int. J. Pharm., 317:40 (2006b).
   Wang et al., Int. J. Pharm., 341:20 (2007).
   Watson et al., Gene Therapy, 16 Feb. 2006, doi: 10.1038/sj.gt.3302735.
   Weller et al., Neurol. Res., 25:611 (2003).
   Westin et al., Eur. J. Pharm. Sci., 24:565 (2005).
   Westin et al., Pharm. Res., 23:565 (2006).
   Whitley et al., Am. J. Med. Genet., 46:209 (1993).
   Williams et al., J. Comp. Neurol., 470:50 (2004).
   Wioland et al., J. Histochem. Cytochem., 48:1215 (2000).
   Wolf et al., Neurobio. Dis., 43:123 (2011).
   Wolf et al., Neurology, 62:1503 (2004)
   Xu et al., J. Clin. Invest., 118:272 (2008).
   Yamada et al., Am. J. Physiol., 261: H1197 (1991).
   Yang et al., J. Pharm. Sci., 94:1577 (2005).
   Zhang et al., Acta Neuropathol. (Berl), 83:233 (1992).
   Zhang et al., Acta Pharmacol. Sin., 25:522 (2004a).
   Zhang et al., Int. J. Pharm., 275:85 (2004b).
   Zhang et al., J. Drug Target, 14:281 (2006).
   Zhao et al., Acta Pharmacol. Sin., 28:273 (2007).
   Zhao et al., Chin. Med. Sci. J., 19:257 (2004).
   Zheng et al., Mol. Genet. Metab., 79:233 (2003).
   Ziegler and Shapiro, In J. Donders and S. Hunter (ed.), Cambridge University Press, p. 427 (2007).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent or inhibit neurocognitive deterioration, to enhance neurocognition, or recover neurologic function in a mammal manifesting or at risk of having Batten's disease, comprising: administering to the central nervous system of the mammal a composition comprising an amount of a recombinant adeno-associated virus (rAAV) vector comprising an open reading frame encoding a gene product of ceroid lipofuscinosis protein (CLN) 2 effective to prevent or inhibit neurocognitive deterioration, enhance neurocognition, or recover neurologic function.

2. The method of claim 1 wherein the mammal is a human.

3. The method of claim 1, wherein the mammal is not an adult.

4. The method of claim 2, wherein the human is: i) about 6 to about 13 years old; ii) about 4 months to about 5 years old; or iii) less than 2.5 years old.

5.-6. (canceled)

7. The method of claim 2, wherein the human has received an enzyme replacement therapy.

8.-13. (canceled)

14. The method of claim 1, wherein the mammal is not treated with an immunosuppressant.

15. The method of claim 1, wherein the mammal is treated with an immunosuppressant.

16. The method of claim 15, wherein the rAAV and the immune suppressant are co-administered or the immune suppressant is administered after the rAAV.

17. The method of claim 1, wherein the mammal: i) is not immunotolerized prior to administration of rAAV; or ii) is immunotolerized prior to administration of rAAV.

18. (canceled)

19. The method of claim 1, wherein the mammal is immunocompetent.

20. The method of claim 1, wherein the rAAV vector is a rAAV1, rAAV3, rAAV4, rAAV5, rAAVrh10, or rAAV9 vector.

21.-22. (canceled)

23. The method of claim 1, wherein multiple doses are administered.

24. (canceled)

25. The method of claim 1, wherein the rAAV is rAAV9 or rAAVrh10.

26. The method of claim 1, wherein the rAAV is intrathecally, intracerebrovascularly or intravenously administered.

27. The method of claim 1, wherein the rAAV is administered to the cisterna magna.

28. The method of claim 15, wherein the immune suppressant comprises cyclophosphamide, a glucocorticoid, cytostatic agents including an alkylating agent, an anti-metabolite, a cytotoxic antibiotic, an antibody, an agent active on immunophilin, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent.

29. The method of claim 1, wherein the amount of rAAV administered is about 1.3×10¹⁰ GC/g brain mass to about 6.5×10¹⁰ GC/g brain mass.

30. The method of claim 1, wherein the amount of rAAV administered is about 1×10³ GC per μL to about 1×10¹⁵ GC per μL.

31. The method of claim 1, wherein the amount of rAAV administered is about 1×10¹² to about 5.6×10¹³ GC (flat dose per mammal).

32. The method of claim 1, wherein the rAAV is intrathecally administered.


SEQUENCE LISTING (5 sequences)

SEQ ID NO: 1 - DNA, 20 bp, synthetic construct
  source 1..20: mol_type = other DNA
gccaaaaattatggggacat

SEQ ID NO: 2 - DNA, 22 bp, synthetic construct
  source 1..22: mol_type = other DNA
attccaacacactattgcaatg

SEQ ID NO: 3 - DNA, 27 bp, synthetic construct
  source 1..27: mol_type = other DNA
atgaagccccttgagcatctgacttct

SEQ ID NO: 4 - DNA, 20 bp, synthetic construct
  source 1..20: mol_type = other DNA
tcccttacctcgaccctttt

SEQ ID NO: 5 - DNA, 19 bp, synthetic construct
  source 1..19: mol_type = other DNA
cacaaggtccatggattgc